(19) United States
(12) Patent Application Publication
(10) Pub. No.: US 2011/0281750 A1
(43) Pub. Date: Nov. 17, 2011

(54) Identifying High Risk Clinically Isolated Syndrome Patients

(75) Inventors: Jean-Christophe Corvol, Paris (FR); Daniel Pelletier, Novato, CA (US); Stephen L. Hauser, San Francisco, CA (US); Jorge R. Oksenberg, Palo Alto, CA (US); Sergio Baranzini, Brisbane, CA (US)

(73) Assignee: The Regents of the University of California, a California Corporation, Oakland, CA (US)

(21) Appl. No.: 13/055,684

(22) PCT Filed: Jul. 24, 2009
(86) PCT No.: PCT/US2009/051750

§ 371 Date: Apr. 14, 2011

Related U.S. Application Data
(60) Provisional application No. 61/083,505, filed on Jul. 24, 2008.
(60) Provisional application No. 61/103,215, filed on Oct. 6, 2008.
(60) Provisional application No. 61/108,469, filed on Oct. 24, 2008.

(51) Int. Cl. C12Q 1/68; G01N 21/64; C40B 30/04

(52) U.S. Cl. 506/9; 435/6.11; 435/6.17; 435/6.18; 435/6.12; 435/7.92

(57) ABSTRACT

Disclosed herein are methods and kits for identifying clinically isolated syndrome (CIS) patients at high risk of developing multiple sclerosis (MS).

### CROSS-REFERENCES TO RELATED APPLICATIONS

This application claims the benefit of U.S. Provisional Application No. 61/083,505, filed Jul. 24, 2008, U.S. Provisional Application No. 61/103,215, filed Oct. 6, 2008, and U.S. Provisional Application No. 61/108,469, filed Oct. 24, 2008, all of which are incorporated herein by reference in their entireties and for all purposes.

### STATEMENT AS TO RIGHTS TO INVENTIONS MADE UNDER FEDERALLY SPONSORED RESEARCH AND DEVELOPMENT

The invention was supported, in whole or in part, by a grant from the National Institutes of Health (2R01NS026799). The Government has certain rights in the invention.

### BACKGROUND OF THE INVENTION

Multiple sclerosis (MS) is a common disabling neurologic disease of young adults (1). Most patients with MS initially present with a clinically isolated syndrome (CIS) due to an inflammatory demyelinating insult in the central nervous system (CNS). Approximately one third of CIS patients will progress to clinically definite MS (CDMS) within 1 year after diagnosis and about half will do so after 2 years (2, 3). Although MRI assessment is routinely used to monitor and forecast conversion into MS, its specificity remains moderate (3). It is estimated that about 10% of CIS patients will remain free of further demyelinating attacks and neurological complications even in the presence of radiological evidence of white matter lesions (4). Although structural neuro-imaging studies are invaluable in the diagnosis and clinical surveillance of MS (3, 5), there is currently no biological marker that accurately predicts MS conversion in CIS patients. Individualized early prognosis and prediction of CDMS would be of substantial value because patients at high risk for rapid progression could be offered disease-modifying therapy, an approach shown to be beneficial in early MS (2).

The present invention meets these and other needs in the art.

### BRIEF SUMMARY OF THE INVENTION

The present invention provides methods and kits for identifying clinically isolated syndrome (CIS) patients at high risk of developing multiple sclerosis (MS).

In one aspect, a method is provided for identifying a patient with clinically isolated syndrome (CIS) at high risk of developing multiple sclerosis (MS). The method includes detecting the level of expression of a marker gene within the patient. The marker gene is a marker gene set forth in Table 18, Table 19, Table 1A, Table 2, Table 4, Table 8, or Table 13, or the marker gene includes a nucleic acid sequence of at least 10 nucleotides in length and at least 90% (e.g., 91%, 92%, 93%, 94%, 95%, 96%, 97%, 98%, 99%, or 100%) identity with a contiguous portion of one of SEQ ID NO:1 to SEQ ID NO:1021. The level of expression of the marker gene is then compared to a standard control whereby a differential expression of the marker gene relative to the standard control indicates that the patient is at high risk of developing multiple sclerosis.

In another aspect, a method is provided for identifying a patient with clinically isolated syndrome (CIS) at high risk of developing multiple sclerosis (MS). The method includes detecting the level of expression of a plurality (e.g. a panel or group) of marker genes within the patient. The plurality of marker genes are all or a portion of marker genes listed in Table 18, Table 19, Table 1A, Table 2, Table 4, Table 8, or Table 13, or the plurality of marker genes comprise a nucleic acid of at least 10 nucleotides in length and at least 90% identity with a contiguous region of all or a portion of marker gene sequences listed in Table 18, Table 19, Table 1A, Table 2, Table 4, Table 8, or Table 13. The marker gene sequences listed in Table 18, Table 19, Table 1A, Table 2, Table 4, Table 8, or Table 13 are referenced as SEQ ID numbers. In some embodiments, the plurality of marker genes are all or a portion of marker genes listed in one of Table 18, Table 19, Table 1A, Table 2, Table 4, Table 8, or Table 13, or the plurality of marker genes comprise a nucleic acid of at least 10 nucleotides in length and at least 90% identity with a contiguous region of all or a portion of marker gene sequences listed in one of Table 18, Table 19, Table 1A, Table 2, Table 4, Table 8, or Table 13. In other embodiments, the plurality of marker genes are all marker genes listed in one of Table 18, Table 19, Table 1A, Table 2, Table 4, Table 8, or Table 13, or the plurality of marker genes comprise a nucleic acid of at least 10 nucleotides in length and at least 90% identity with a contiguous region of all marker gene sequences listed in one of Table 18, Table 19, Table 1A, Table 2, Table 4, Table 8, or Table 13. The level of expression of the marker gene to a standard control is compared whereby a differential expression of the marker gene relative to the standard control indicates that the patient is at high risk of developing multiple sclerosis.

In another aspect, a kit is provided for use in identifying a patient with clinically isolated syndrome (CIS) at high risk of developing multiple sclerosis (MS). The kit includes (i) a nucleic acid sequence having at least 90%, 91%, 92%, 93%, 94%, 95%, 96%, 97%, 98%, 99%, or 100% identity over at least a 10 or 20 nucleotide continuous region with one or more nucleic acids within a marker gene identified in Table 18, Table 19, Table 1A, Table 2, Table 4, Table 8, and/or Table 13, (ii) a nucleic acid sequence having at least 90%, 91%, 92%, 93%, 94%, 95%, 96%, 97%, 98%, 99%, or 100% identity over at least a 10 or 20 nucleotide continuous region with a target sequence to which the probe set identified in Table 18, Table 19, Table 1A, Table 2, Table 4, Table 8 and/or Table 13 is designed to interrogate, or (iii) a nucleic acid complimentary to the nucleic acids set forth in (i) or (ii) above. In some embodiments, the kit also includes an electronic device or computer software capable of comparing a marker gene expression level from the patient to a standard control thereby indicating whether the patient is at high risk of developing multiple sclerosis.

### BRIEF DESCRIPTION OF THE DRAWINGS

FIG. 1. Molecular signature in CD4+ cells segregates CIS patients from controls. A. Three dimensional plot of the first 3 principal components computed from expression values of the 1,718 genes with the highest variance across all samples. B. Hierarchical clustering of expression values from the same genes and samples as in A. C. Gene ontology (GO) categories significantly enriched in CIS patients at baseline. D. A representative model for the prediction of the 4 CIS groups using the Integrated Bayesian Inference System (IBIS).

FIG. 2. Clinical and radiological characteristics of the 4 CIS groups. A. Clinical and MRI characteristics of the 4 CIS groups defined by gene expression. B. Hierarchical clustering of differences (delta) between measurements at baseline and 12 months later of brain parenchyma (nBPV), grey matter (nGMV), and white matter (nWMV), CSF (nCSFV) volumes, and SIENA (PBVC) expressed as quartiles. C. Kaplan-Meyer curve of high risk (red) and low risk (blue) groups of MS conversion as predicted by the expression of RNA gene products hybridizing to 28 probe sets set forth in Table 2. D. Hierarchical clustering of the mRNA gene products hybridizing to 108 probe sets set forth in Table 1A that characterize group #1 patients.

FIG. 3. TOB1 abrogates T cell quiescence. A. Relative expression (fold change compared to controls) of TOB1 in CIS patients from group #1 and CIS patients from other groups assessed by RT-PCR. B. Relative expression of TOB1 (yellow bars), Interleukin-2 (green bars) and Interferon-gamma (red bars) assessed by RT-PCR in CD4+ T cells cultured for 6 or 24 hours in plates coated with 1 mg/ml anti-CD3 and 1 mg/ml anti-CD28 antibodies (n=3). C. Immunostaining for TOB1 and CD4 in lymph nodes of mice injected with MOG₃₅₋₅₅, CFA alone, or vehicle. D. Microarray-based TOB1 expression in lymph nodes and spinal cords from mice immunized with MOG₃₅₋₅₅+ adjuvant (EAE) or adjuvant only (CFA). E. Relative expression (fold change compared to controls) of CD44 in CIS patients from group #1 and CIS patients from other groups assessed by RT-PCR. F. Plasma OPN concentration in group #1 patients, other CIS and controls measured by ELISA. G. Genomic map of TOB1 showing the relative position of the 5 SNP used for association analysis. H. Schematic representation of gene expression signature in T cells from group #1 patients.

FIG. 4. Gene expression still differentiates group #1 from other CIS patients a year later. A. Hierarchical clustering of the expression of the same 1,718 genes as in FIG. 1 but obtained at 12 months. B. Number of genes differentially expressed in CIS compared to controls at baseline (orange circle), at 12 months (blue circle) or on both sets (intersection). C. A SVM predictive model was built using the expression of mRNA gene products hybridizing to 108 probe sets set forth in Table 1A that distinguished group #1 from other CIS patients.

### DETAILED DESCRIPTION OF THE INVENTION

### I. Definitions

Unless defined otherwise, all technical and scientific terms used herein have the same meaning as commonly understood by one of ordinary skill in the art to which this invention belongs. Generally, the nomenclature used herein and the laboratory procedures in cell culture, molecular genetics, and nucleic acid chemistry and hybridization described below are those well known and commonly employed in the art.

As used herein, “nucleic acid” means either DNA, RNA, single-stranded, double-stranded, or more highly aggregated hybridization motifs, and any chemical modifications thereof. Modifications include, but are not limited to, those which provide other chemical groups that incorporate additional charge, polarizability, hydrogen bonding, electrostatic interaction, and functionality to the nucleic acid ligand bases or to the nucleic acid ligand as a whole. Such modifications include, but are not limited to, peptide nucleic acids, phosphodiester group modifications (e.g., phosphorothioates, methylphosphonates), 2′-position sugar modifications, 5-position pyrimidine modifications, 8-position purine modifications, modifications at exocyclic amines, substitution of 4-thiouridine, substitution of 5-bromo or 5-iodo-uracil; backbone modifications, methylations, unusual base-pairing combinations such as the isobases isocytidine and isoguanidine and the like. Modifications can also include 3′ and 5′ modifications such as capping.

The term “nucleic acid” or “polynucleotide” refers to deoxyribonucleotides or ribonucleotides and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs (haplotypes), and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Cassol et al. (1992); Rossolini et al., Mol. Cell. Probes 8:91-98 (1994)). The term nucleic acid is used interchangeably with gene, cDNA, and mRNA encoded by a gene.

The phrase “selectively (or specifically) hybridizes to” refers to the detectable binding, duplexing, or hybridizing of a molecule only to a particular nucleotide sequence under stringent hybridization conditions when that sequence is present in a complex mixture (e.g., total cellular or library DNA or RNA).

The terms “identical” or percent “identity,” in the context of two or more nucleic acids, refer to two or more sequences or subsequences that are the same or have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher) identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., the NCBI web site or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 10, 11, 12, 13, 14, 15, 20, 25 amino acids or nucleotides in length, or over a region in the range 10-20, 10-25, 10-30, 10-40, 10-50, 10-60, 10-70, 10-80, 10-90, or even 10-100. In certain preferred embodiments, identity exists over a region that is 10-100 amino acids or nucleotides in length.

The phrase “stringent hybridization conditions” refers to conditions under which a first nucleic acid will hybridize to its target subsequence, typically in a complex mixture of nucleic acid, but not detectably to other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For selective or specific hybridization, a positive signal is at least two times background, optionally 10 times background hybridization. Exemplary stringent hybridization conditions can be as following: 50% formamide, 5×SSC, and 1% SDS, incubating at 42° C., or 5×SSC, 1% SDS, incubating at 65° C., with wash in 0.2×SSC, and 0.1% SDS at 65° C. Such washes can be performed for 5, 15, 30, 60, 120, or more minutes.

Exemplary “moderately stringent hybridization conditions” include a hybridization in a buffer of 40% formamide, 1 M NaCl, 1% SDS at 37° C., and a wash in 1×SSC at 45° C. Such washes can be performed for 5, 15, 30, 60, 120, or more minutes. A positive hybridization is at least twice background. Those of ordinary skill will readily recognize that alternative hybridization and wash conditions can be utilized to provide conditions of similar stringency.

The terms “differential expression” or “differentially expressed” used in reference to the expression of a marker gene means an elevated level of expression of the marker gene or a lowered level of expression of the marker gene relative to a standard control that is indicative of a high risk CIS patient, as set forth in the methods and results disclosed herein (e.g. Tables 1, 2, 10A-12C, and 15A-17C). As is customary in the art, marker genes described herein each have an associated name (e.g., C17orf65, C4orf10, FAM98A, and the like). Accordingly, reference to a marker gene name in turn refers to the marker gene itself. “Target sequence” refers to a region within a target gene (e.g., marker gene) which a probe will identify, as known in the art. The term “probe set identifier” refers to set of nucleic acid probes capable of identifying a particular marker gene (e.g. target sequence). Probe set identifiers may be provided by Affymetrix (Santa Clara, Calif.), for example, as known in the art and disclosed herein. It is understood that one of skill in the art can, with only routine experimentation, design and use probes to identify specific marker genes as described herein. It is further understood that more than one probe, and more than one probe set identifier may be designed to identify a specific gene, for example a marker gene described herein.

### II. Methods and Kits

Provided herein are methods of determining whether a patient with clinically isolated syndrome (CIS) is at high risk of developing multiple sclerosis (MS). CIS patients at high risk of developing MS are typically those patients that develop MS within two years of being initially diagnosed with CIS or within two years of the onset of CIS. In some embodiments, high risk CIS patients are those that develop CIS within 18 months, 12 months, or 9 months of being initially diagnosed with CIS or the onset of CIS. Thus, the methods provided herein are useful in identifying CIS patients that are likely to develop MS quickly relative to the average CIS patient.

It has been discovered that certain genes are markers of rapid development of MS in CIS patients. These marker genes were identified as genes that are differentially expressed relative to healthy individuals and/or CIS patients that do not develop MS quickly (i.e. those that are at low risk of rapid onset of MS). Thus, by detecting the level of expression of a marker gene within a CIS patient and comparing the level of expression of the maker gene to a standard control, high risk CIS patients may be identified. In some embodiments, the level of a plurality (e.g. a panel) of marker genes are detected and compared to the level of expression of the maker gene to a standard control to identify high risk CIS patients. Specific panels or groups of maker genes are discussed below. In some embodiments, the standard control may be approximately the average amount of expression of the marker gene(s) in humans, humans without CIS, or humans with CIS that are not at high risk of developing MS. In other embodiments, the standard control is a detected level of expression of a standard control gene in the CIS patient.

In some embodiments, the marker gene is a gene set forth in Table 18, Table 19, Table 1A, Table 2, Table 4, Table 8 and/or Table 13. In some embodiments, the marker gene is any one of the genes set forth in Table 18, Table 19, Table 1A, Table 2, Table 4, Table 8 and/or Table 13. In some embodiments, a plurality (e.g. a panel or group) of marker genes are detected. Thus, in some embodiments all the marker genes set forth in one of the following tables is selected: Table 18, Table 19, Table 1A, Table 2, Table 4, Table 8 or Table 13. In another embodiment, at least 2-9, 10, 20, 30, 40, 50, 60, 70, 80, 90, 100, 150, 200, 250, 300, 400, 500, 600, 700, or 800 of the marker genes set forth in one of the following tables is selected, as appropriate according to the number of genes set forth within the following tables: Table 18, Table 19, Table 1A, Table 2, Table 4, Table 8 or Table 13. Where a plurality (e.g. panel or group) of genes are detected, any combination of the marker genes disclosed in relevant table(s) may be detected. By measuring the expression levels of these one or more of these marker genes in a patient with CIS and comparing those levels with healthy individuals and/or CIS patients that do not develop MS quickly, the risk of the patient developing MS may be assessed. In some related embodiments, the marker gene is ZNF12, C17orf65, BAT1, ARHGDIA, NAPA, ATP5G2, DDX52, NDFIP1, SDAD1, USP7, MEF2A, AGER, RAB1B, GDI1 and/or BANF1. In other related embodiments, the marker gene is ZNF12, C17orf65, BAT1, ARHGDIA, NAPA, ATP5G2, DDX52, NDFIP1 and/or SDAD1. In still other related embodiments, the marker gene is USP7, MEF2A, AGER, RAB1B, GDI1 and/or BANF1. In other embodiments, the marker gene is TOB1. In other embodiments, the marker gene is not TOB1. In some embodiments, the marker gene is C17orf65, C4orf10, FAM98A, TLE1, INHBC, NAPA, TKT, TPT1, FLJ20054, KIAA0794, LOC 134492, and/or MGC34648. In some embodiments, the marker gene is any one of C17orf65, C4orf10, FAM98A, TLE1, INHBC, NAPA, TKT, TPT1, FLJ20054, KIAA0794, LOC134492 or MGC34648. In some embodiments, the marker gene is included within a plurality of genes selected from C17orf65, C4orf10, FAM98A, TLE1, INHBC, NAPA, TKT, TPT1, FLJ20054, KIAA0794, LOC134492 and MGC34648. In some embodiments, the marker gene is CD1D, CD44, CDC34, CDKN1C, CD47, GZMM, and/or PPIA. In some embodiments, the marker gene is any one of CD1D, CD44, CDC34, CDKN1C, CD47, GZMM, or PPIA. In some embodiments, the marker gene included within a plurality of genes selected from CD1D, CD44, CDC34, CDKN1C, CD47, GZMM, or PPIA.

In certain embodiments, the method described herein for detecting the level of expression of a marker gene is an in vitro method. In some embodiments, the marker gene is a gene set forth in Table 18, Table 19, Table 1A, Table 2, Table 4, Table 8 and/or Table 13, and detection is conducted in vitro (e.g. on a biological sample derived from a CIS patient).

The expression levels of the marker genes may be measured using any appropriate method. In some embodiments, the amount of RNA expressed by the marker gene is measured. The amount of RNA expressed may be assessed, for example, using nucleic acid probes with marker gene coding sequences or using quantitative PCR techniques. For example, a nucleic acid array forming a probe set may be used to detect RNA expressed by the marker gene. The RNA expressed by the marker gene may be transcribed to cDNA (and in some cases to cRNA) and then queried with a gene chip array using methods known in the art. Thus, in some embodiments the marker gene may also be a gene including a nucleic acid sequence having at least 90%, 91%, 92%, 93%, 94%, 95%, 96%, 97%, 98%, 99%, or 100% identity over at least a 10 or 20 nucleotide continuous region (i.e. sequence) within a marker gene identified in Table 18, Table 19, Table 1A, Table 2, Table 4, Table 8, and/or Table 13, or with a target sequence to which the probe set identified in Table 18, Table 19, Table 1A, Table 2, Table 4, Table 8 and/or Table 13 is designed to interrogate. For example, the continuous region may be 10, 11, 12, 13, 14, 15, 16, 17, 18, 19 or 20 nucleotides in length. In related embodiments, the marker gene includes a nucleic acid sequence having at least 90%, 91%, 92%, 93%, 94%, 95%, 96%, 97%, 98%, 99%, or 100% identity with the entire length of one or more nucleic acids within a marker gene identified in Table 18, Table 19, Table 1A, Table 2, Table 4, Table 8, and/or Table 13, or with a target sequence to which the probe set identified in Table 18, Table 19, Table 1A, Table 2, Table 4, Table 8 and/or Table 13 is designed to interrogate. In other related embodiments, the marker gene includes a nucleic acid sequence having 100% identity with the entire length of one or more nucleic acids within a marker gene identified in Table 18, Table 19, Table 1A, Table 2, Table 4, Table 8, and/or Table 13, or with a target sequence to which the probe set identified in Table 18, Table 19, Table 1A, Table 2, Table 4, Table 8 and/or Table 13 is designed to interrogate. In other related embodiments, “one or more” nucleic acids within a probe set identified in Table 18, Table 19, Table 1A, Table 2, Table 4, Table 8, and/or Table 13 referred to above is the majority or all of the nucleic acids within the probe set.

Tables 1A, 2, 4, 8, 13, 18 and 19 provide probe set identifiers using Affymetrix probe set identifier numbers, as known in the art. The nucleic acid sequences contained within each probe set identifier number and the target sequence to which the probe set is designed to interrogate are publicly available in a variety of sources, including the Affymetrix website and the National Cancer Institute website. The term “designed to interrogate” in the context of target genes, marker genes and probes refers to a probe having sufficient primary sequence complementarity to a target to detectably bind the target, as well known in the art.

In some embodiments, the marker gene includes a nucleic acid sequence within a marker gene identified in Table 1A. In other embodiments, the marker gene includes a nucleic acid sequence within a marker gene identified in Table 2. In other embodiments, the marker gene includes a nucleic acid sequence within a p marker gene identified in Table 4. In other embodiments, the marker gene includes a nucleic acid sequence within a marker gene identified in Table 8. In other embodiments, the marker gene includes a nucleic acid sequence within a marker gene identified in Table 13. In some embodiments, the marker gene is a gene set forth in Table 18. In some embodiments, the marker gene is C17orf65 (SEQ ID NO:977), C4orf10 (SEQ ID NO:1005), FAM98A (SEQ ID NO:1020), TLE1 (SEQ ID NO:844), INHBC (SEQ ID NO:993), NAPA (SEQ ID NO:995), TKT (SEQ ID NO:994), TPT1 (SEQ ID NO:138), F1120054 (SEQ ID NO:11), KIAA0794 (SEQ ID NO:104), LOC134492 (SEQ ID NO:184), and/or MGC34648 (SEQ ID NO:348). In some embodiments, the expression levels of a plurality (e.g., 2, 3, 4, 5, 6, 7, 8, 9, 10, 11, 12 and/or 13) of marker genes as disclosed in Table 18 are detected. In some embodiments, the marker gene is a gene set forth in Table 19. In some embodiments, the marker gene is CD1D (SEQ ID NO:376), CD44 (SEQ ID NO:275), CDC34 (SEQ ID NO:553), CDKN1C (SEQ ID NO:320), CD47 (SEQ ID NO:1015), GZMM (SEQ ID NO:617), and/or PPIA (SEQ ID NO:1010). In some embodiments, the expression levels of a plurality (e.g., 2, 3, 4, 5, 6 or 7) of marker genes as disclosed in Table 19 are detected.

The comparison of the marker gene expression levels with a standard control may be accomplished by determining whether the marker gene is expressed in the CIS patient at an elevated level or a lowered level (i.e. detecting differential expression). The elevated or lowered levels are indicative of rapid development of multiple sclerosis (MS) (e.g. within two years of being initially diagnosed with CIS). Whether elevation or lowering of expression of a particular marker gene expression is indicative of rapid onset of MS in a CIS patient is clearly set forth in Tables 1A, 2, 10A-12C, and 15A-17C. For example, where the marker gene is TOB1, Table 1A clearly shows that lowered expression of TOB1 is indicative of rapid onset of MS in a CIS patient.

The standard control may be any appropriate standard known in the art. In some embodiments, the standard control is approximately the average amount of expression of the marker gene in humans, humans without CIS, or humans with CIS that are not at high risk of developing MS. Approximate average relative amounts of expression of marker genes are set forth in Tables 1A and 2 in a sample of humans without CIS, humans with CIS that are not at high risk of developing MS, and humans with CIS at high risk of developing MS. In addition, Table 4 provides approximate average amounts of expression of genes for humans with CIS and humans without CIS.

In other embodiments, the standard control is a detected level of expression of a standard control gene in the CIS patient. As used herein, a standard control gene is a human gene that is expressed at approximately constant levels thereby providing a baseline reading of gene expression for an individual. The standard control gene may also be referred to herein and in the art as a housekeeping gene. In some embodiments, the standard control gene is GAPDH, 18s ribosomal subunit, beta actin (ACTB), PPP1CA, beta 2 microglobulin (B2M), HPRT1, RPS13, RPL27, RPS20 or OAZ1.

The elevated level of expression of the marker gene or the lowered level of expression of the marker gene may be determined by calculating the ratio of the level of expression of the marker gene to the level of expression of a standard control gene. For example, Table 1B lists an average amount of GAPDH in the subjects studied according to the examples set forth below. The corresponding ratios of marker genes to GAPDH are set forth in Tables 1A and 2. By using the calculated ratios provided in Tables 1A and 2, the ratio of expression of a corresponding marker gene to GAPDH in a CIS patient may be calculated. Where the calculated marker to GAPDH ratio in the patient is approximately equal to the corresponding ratio provided in Table 1A and 2, the CIS patient is at high risk of rapidly developing MS.

In some embodiments, statistical models are established for determining whether expression of a marker gene is indicative of a CIS patient that is highly likely to develop MS, for example within 9 months of being initially diagnosed with CIS. Thus, in some related embodiments, the standard control is a threshold expression value obtained from a statistical model. Threshold expression values may be obtained optionally using a standard gene (e.g. GADPH or ACTB) and a classifier algorithm (e.g. compound covariate predictor (CCP), diagonal linear discriminant analysis (DLDA), and/or support vector machines (SVM) classifiers) (see Example 9 and Tables 8 to 17C). In some embodiments, a composite predictor is used to establish a statistical model or threshold vale wherein the composite predictor employs a CCP, DLDA and SVM. Where the expression of a marker gene in a CIS subject is above the calculated threshold expression value, a patient with CIS is at high risk for developing MS.

Using the teachings provided herein, one skilled in the art is enabled to use any known housekeeping gene to establish similar ratios and statistical models to identify CIS patients at high risk of rapidly developing MS using the disclosed methods.

In another aspect, there is provided an in vitro method for determining whether a patient with clinically isolated syndrome (CIS) is at high risk of developing multiple sclerosis (MS). The method includes isolating mRNA from the patient, thereby providing an in vitro nucleic acid sample. Optionally, the method further includes subjecting the in vitro nucleic acid sample to polymerase chain reaction under conditions suitable to amplify nucleic acid within the in vitro nucleic acid sample. The in vitro nucleic acid sample is contacted with a microarray, the microarray having a plurality of probes designed to interrogate specific marker genes. The level of nucleic acid duplex formation is determined between the in in vitro nucleic acid sample and the microarray, thereby providing the expression level of nucleic acid present in the in vitro nucleic acid sample. The expression level of nucleic acid is then compared to the expression level of a standard control. A differential expression of the marker gene relative to said standard control indicates that the patient is at high risk of developing multiple sclerosis. In some embodiments, the standard control may be approximately the average amount of expression of the marker gene in humans, humans without CIS, or humans with CIS that are not at high risk of developing MS. In other embodiments, the standard control is a detected level of expression of a standard control gene in the CIS patient. In some embodiments, the marker gene is a gene set forth in Table 18, Table 19, Table 1A, Table 2, Table 4, Table 8 and/or Table 13. In some embodiments, the marker gene is any one of the marker genes set forth in Table 18, Table 19, Table 1A, Table 2, Table 4, Table 8 and/or Table 13. In some embodiments, the expression level of a plurality (e.g., 2, 3, 4, 5, 6, 7, 8, 9, 10, 15, 20, 30, 40, 50, 60, 70, 80, 90 or 100) of marker genes as set forth in Table 18, Table 19, Table 1A, Table 2, Table 4, Table 8 and/or Table 13, are determined. In some embodiments, the marker gene is ZNF12, C17orf65, BATT, ARHGDIA, NAPA, ATP5G2, DDX52, NDFIP1, SDAD1, USP7, MEF2A, AGER, RAB1 B, GDI1 and/or BANF1. In other related embodiments, the marker gene is ZNF12, C17orf65, BAT1, ARHGDIA, NAPA, ATP5G2, DDX52, NDFIP1 and/or SDAD1. In still other related embodiments, the marker gene is USP7, MEF2A, AGER, RAB1 B, GDI1 and/or BANF1. In some embodiments, the expression levels of a plurality (e.g., 2, 3, 4, 5, 6, 7, 8, 9, 10, 11, 12, 13, 14 or 15) of marker genes selected from ZNF12, C17orf65, BAT1, ARHGDIA, NAPA, ATP5G2, DDX52, NDFIP1, SDAD1, USP7, MEF2A, AGER, RAB1 B, GDI1 and BANF1. In some embodiments, the marker gene is a gene set forth in Table 18. In some embodiments, the marker gene is C17orf65 (SEQ ID NO:977), C4orf10 (SEQ ID NO:1005), FAM98A (SEQ ID NO:1020), TLE1 (SEQ ID NO:844), INHBC (SEQ ID NO:993), NAPA (SEQ ID NO:995), TKT (SEQ ID NO:994), TPT1 (SEQ ID NO:138), F1120054 (SEQ ID NO:11), KIAA0794 (SEQ ID NO:104), LOC134492 (SEQ ID NO:184), or MGC34648 (SEQ ID NO:348). In some embodiments, the expression levels of a plurality (e.g., 2, 3, 4, 5, 6, 7, 8, 9, 10, 11, 12 or 13) of marker genes as disclosed in Table 18 are detected. In some embodiments, the marker gene is a gene set forth in Table 19. In some embodiments, the marker gene is CD1D (SEQ ID NO:376), CD44 (SEQ ID NO:275), CDC34 (SEQ ID NO:553), CDKN1C (SEQ ID NO:320), CD47 (SEQ ID NO:1015), GZMM (SEQ ID NO:617), or PPIA (SEQ ID NO:1010). In some embodiments, the expression levels of a plurality (e.g., 2, 3, 4, 5, 6 or 7) of marker genes as disclosed in Table 19 are detected.

In another aspect, a kit is provided for use in identifying a patient with clinically isolated syndrome (CIS) at high risk of developing multiple sclerosis (MS). The kit includes (i) a nucleic acid sequence having at least 90%, 91%, 92%, 93%, 94%, 95%, 96%, 97%, 98%, 99%, or 100% identity over at least a 10 nucleotide continuous region (e.g., 10, 11, 12, 13, 14, 15, 16, 17, 18, 19 or 20, or within the range 10-50, 10-40, 10-30, or 10-20) with one or more nucleic acids within a marker gene identified in Table 18, Table 19, Table 1A, Table 2, Table 4, Table 8, and/or Table 13, (ii) a nucleic acid sequence having at least 90%, 91%, 92%, 93%, 94%, 95%, 96%, 97%, 98%, 99%, or 100% identity over at least a 10 (e.g., 10, 11, 12, 13, 14, 15, 16, 17, 18, 19 or 20) nucleotide continuous region with a target sequence to which the probe set identified in Table 18, Table 19, Table 1A, Table 2, Table 4, Table 8 and/or Table 13 is designed to interrogate, or (iii) a nucleic acid complimentary to the nucleic acids set forth in (i) or (ii) above. In some embodiments, the kit also includes an electronic device or computer software capable of comparing a marker gene expression level from the patient to a standard control thereby indicating whether the patient is at high risk of developing multiple sclerosis. In some embodiments, the kit contains a plurality (e.g., 2, 3, 4, 5, 6, 7, 8, 9, 10, 11, 12, 13, 14, 15, 16, 17, 18, 19 or 20) of nucleic acid sequences having at least 90%, 91%, 92%, 93%, 94%, 95%, 96%, 97%, 98%, 99%, or 100% identity over at least a 10 nucleotide continuous region (e.g., 10, 11, 12, 13, 14, 15, 16, 17, 18, 19 or 20, or within the range 10-50, 10-40, 10-30, or 10-20) with a marker gene identified in Table 18, Table 19, Table 1A, Table 2, Table 4, Table 8, and/or Table 13, or complement thereof. In some embodiments, the kit contains a plurality (e.g., 2, 3, 4, 5, 6, 7, 8, 9, 10, 11, 12, 13, 14, 15, 16, 17, 18, 19 or 20) of nucleic acid sequences having at least 90%, 91%, 92%, 93%, 94%, 95%, 96%, 97%, 98%, 99%, or 100% identity over at least a 10 nucleotide continuous region (e.g., 10, 11, 12, 13, 14, 15, 16, 17, 18, 19 or 20, or within the range 10-50, 10-40, 10-30, or 10-20) with a marker gene identified in Table 18, Table 19, Table 1A, Table 2, Table 4, Table 8, and/or Table 13, or complement thereof. In some embodiments, the plurality of marker genes are all or a portion of marker genes listed in Table 18, Table 19, Table 1A, Table 2, Table 4, Table 8, or Table 13, or the plurality of marker genes comprise a nucleic acid of at least 10 nucleotides in length and at least 90% identity with a contiguous region of all or a portion of marker gene sequences listed in Table 18, Table 19, Table 1A, Table 2, Table 4, Table 8, or Table 13. In some embodiments, the plurality of marker genes are all or a portion of marker genes listed in one of Table 18, Table 19, Table 1A, Table 2, Table 4, Table 8, or Table 13, or the plurality of marker genes comprise a nucleic acid of at least 10 nucleotides in length and at least 90% identity with a contiguous region of all or a portion of marker gene sequences listed in one of Table 18, Table 19, Table 1A, Table 2, Table 4, Table 8, or Table 13. In other embodiments, the plurality of marker genes are all marker genes listed in one of Table 18, Table 19, Table 1A, Table 2, Table 4, Table 8, or Table 13, or the plurality of marker genes comprise a nucleic acid of at least 10 nucleotides in length and at least 90% identity with a contiguous region of all marker gene sequences listed in one of Table 18, Table 19, Table 1A, Table 2, Table 4, Table 8, or Table 13.

In some embodiments, the electronic device or computer software employs the use of a statistical model. The electronic device or computer software may also utilize a threshold expression values obtained optionally using a standard gene (e.g. GADPH or ACTB) and a classifier algorithm (e.g. compound covariate predictor (CCP), diagonal linear discriminant analysis (DLDA), and/or support vector machines (SVM) classifiers) such as those set forth in Example 9 and Tables 8 to 17. One skilled in the art will immediately recognize that the electronic device or computer software may be used in the methods disclosed herein.

In some embodiments, the nucleic acid provided in the kit above may be a probe nucleic acid for use in a PCR technique, such as quantitative PCR, to assess the expression of a given marker gene. In some embodiments, the nucleic acid sequence has 100% identity with a continuous nucleic acid region (i.e. sequence) within a marker gene identified in Table 18, Table 19, Table 1A, Table 2, Table 4, Table 8, and/or Table 13, or with a target sequence to which the probe set identified in Table 18, Table 19, Table 1A, Table 2, Table 4, Table 8 and/or Table 13 is designed to interrogate, or is complimentary thereto. In other embodiments, the nucleic acid has the same sequence as a nucleic acid contained within a marker gene identified in Table 18, Table 19, Table 1A, Table 2, Table 4, Table 8, and/or Table 13 or the target sequence to which the probe set identified in Table 18, Table 19, Table 1A, Table 2, Table 4, Table 8 and/or Table 13 is designed to interrogate, or is complimentary thereto.

The nucleic acid provided in the kit may also hybridize under stringent conditions (or moderately stringent conditions) to a nucleic acid sequence within a marker gene identified in Table 18, Table 19, Table 1A, Table 2, Table 4, Table 8 and/or Table 13 or a target sequence to which the probe set identified in Table 18, Table 19, Table 1A, Table 2, Table 4, Table 8 and/or Table 13 is designed to interrogate. The nucleic acid provided in the kit may also be perfectly complimentary to a nucleic acid sequence within a marker gene identified in Table 18, Table 19, Table 1A, Table 2, Table 4, Table 8 and/or Table 13 or a target sequence to which the probe set identified in Table 18, Table 19, Table 1A, Table 2, Table 4, Table 8 and/or Table 13 is designed to interrogate.

The present invention also contains the subject matter of the following numbered embodiments:

### Embodiment 1

A method of identifying a patient with clinically isolated syndrome (CIS) at high risk of developing multiple sclerosis (MS), said method comprising:
detecting the level of expression of a marker gene within said patient, wherein said marker gene is a marker gene set forth in Table 18, Table 19, Table 1A, Table 2, Table 4, Table 8, or Table 13, or said marker gene comprises a nucleic acid of at least 10 nucleotides in length and at least 90% identity with a contiguous portion of one of SEQ ID NO:1 to SEQ ID NO:1021; and comparing the level of expression of said marker gene to a standard control whereby a differential expression of said marker gene relative to said standard control indicates that said patient is at high risk of developing multiple sclerosis.

### Embodiment 1A

A method of identifying a patient with clinically isolated syndrome (CIS) at high risk of developing multiple sclerosis (MS), said method comprising:
detecting the level of expression of a plurality of marker genes within said patient, wherein said plurality of marker genes are all or a portion of marker genes listed in one of Table 18, Table 19, Table 1A, Table 2, Table 4, Table 8, or Table 13, or said plurality of marker genes comprises a nucleic acid of at least 10 nucleotides in length and at least 90% identity with a contiguous region of all or a portion of marker gene sequences listed in one of Table 18, Table 19, Table 1A, Table 2, Table 4, Table 8, or Table 13; and
   comparing the level of expression of said plurality of marker genes to a standard control whereby a differential expression of said plurality of marker genes relative to said standard control indicates that said patient is at high risk of developing multiple sclerosis.

### Embodiment 2

The method of Embodiment 1, wherein said marker gene comprises a nucleic acid sequence at least 10 nucleotides in length having at least 90% identity with a contiguous portion of a nucleic acid having the sequence of one of SEQ ID NO:1 to SEQ ID NO:1021.

### Embodiment 3

The method of Embodiment 1 or Embodiment 2, wherein the said marker gene comprises a nucleic acid sequence at least 10 nucleotides in length having at least 95% identity with a contiguous portion of a nucleic acid having the sequence of one of SEQ ID NO:1 to SEQ ID NO:1021.

### Embodiment 4

The method of any preceding Embodiments, wherein the method is an in vitro method and comprises detecting the level of expression of a marker gene in a sample previously isolated from said patient.

### Embodiment 5

The method of Embodiment 4, which comprises contacting the sample with at least one_nucleic acid of at least 10 nucleotides in length and having at least 90% identity with a contiguous portion of one of SEQ ID NO:1 to SEQ ID NO:1021, and optionally comprises contacting the sample with 2, 3, 4, 5, 6, 7, 8, 9, 10 or more nucleic acids of at least 10 nucleotides in length and having at least 90% identity with a contiguous portion of one of SEQ ID NO:1 to SEQ ID NO:1021.

### Embodiment 6

The method of Embodiment 4, which comprises contacting the sample with at least one_nucleic acid of at least 10 nucleotides in length and at least 95% identity with a contiguous portion of one of SEQ ID NO:1 to SEQ ID NO:1021, and optionally comprises contacting the sample with 2, 3, 4, 5, 6, 7, 8, 9, 10 or more nucleic acids of at least 10 nucleotides in length and having at least 95% identity with a contiguous portion of one of SEQ ID NO:1 to SEQ ID NO:1021,

### Embodiment 7

The method of Embodiment 4, which comprises contacting the sample with at least one_nucleic acid of at least 10 nucleotides in length and at least 99% identity with a contiguous portion of one of SEQ ID NO:1 to SEQ ID NO:1021, and optionally comprises contacting the sample with 2, 3, 4, 5, 6, 7, 8, 9, 10 or more nucleic acids of at least 10 nucleotides in length and having at least 99% identity with a contiguous portion of one of SEQ ID NO:1 to SEQ ID NO:1021,

### Embodiment 8

The method of any preceding Embodiment, wherein said marker gene is a marker gene set forth in Table 18.

### Embodiment 9

The method of any of Embodiments 1 to 7, wherein said marker gene is a marker gene set forth in Table 19.

### Embodiment 10

The method of any of Embodiments 1 to 7, wherein said marker gene is ZNF12 (SEQ ID NO:83), C17orf65 (SEQ ID NO:977), BAT1 (SEQ ID NO:981), ARHGDIA (SEQ ID NO:1000), NAPA (SEQ ID NO:995), ATP5G2 (SEQ ID NO:996), DDX52 (SEQ ID NO:292), NDFIP1 (SEQ ID NO:2), SDAD1 (SEQ ID NO:116), USP7 (SEQ ID NO:1014), MEF2A (SEQ ID NO:1007), AGER (SEQ ID NO:998), RAB1B (SEQ ID NO:1011), GDI1 (SEQ ID NO:986) or BANF1(SEQ ID NO:999).

### Embodiment 11

The method of any of Embodiments 1 to 7, wherein said marker gene is ZNF12 (SEQ ID NO:83), C17orf65 (SEQ ID NO:977), BAT1 (SEQ ID NO:981), ARHGDIA (SEQ ID NO:1000), NAPA (SEQ ID NO:995), ATP5G2 (SEQ ID NO:996), DDX52 (SEQ ID NO:292), NDFIP1 (SEQ ID NO:2) or SDAD1 (SEQ ID NO:116).

### Embodiment 12

The method of any of Embodiments 1 to 7, wherein said marker gene is USP7 (SEQ ID NO:1014), MEF2A (SEQ ID NO:1007), AGER (SEQ ID NO:998), RAB1B (SEQ ID NO:1011), GDI1 (SEQ ID NO:986) or BANF1 (SEQ ID NO:999).

### Embodiment 13

The method of any of Embodiments 1 to 7, wherein said marker gene is C17orf65 (SEQ ID NO:977), C4orf10 (SEQ ID NO:1005), FAM98A (SEQ ID NO:1020), TLE1 (SEQ ID NO:844), INHBC (SEQ ID NO:993), NAPA (SEQ ID NO:995), TKT (SEQ ID NO:994), TPT1 (SEQ ID NO:138), F1120054 (SEQ ID NO:11), KIAA0794 (SEQ ID NO:104), LOC134492 (SEQ ID NO:184), or MGC34648 (SEQ ID NO:348).

### Embodiment 14

The method of any of Embodiments 1 to 7, wherein said marker gene is CD1D (SEQ ID NO:376), CD44 (SEQ ID NO:275), CDC34 (SEQ ID NO:553), CDKN1C (SEQ ID NO:320), CD47 (SEQ ID NO:1015), GZMM (SEQ ID NO:617), or PPIA (SEQ ID NO:1010).

### Embodiment 15

The method of any preceding Embodiment, wherein said standard control is a detected level of expression of a standard control gene in said patient.

### Embodiment 16

The method of Embodiment 15, wherein said standard control gene is GAPDH, 18s ribosomal subunit, beta actin (ACTB), PPP1CA, beta 2 microglobulin (B2M), HPRT1, RPS13, RPL27, RPS20 or OAZ1.

### Embodiment 17

The method of Embodiment 16, wherein said standard control gene is GAPDH.

### Embodiment 18

The method of any preceding Embodiment, wherein the elevated level of expression of said marker gene or the lowered level of expression of said marker gene is determined by the ratio of the level of expression of said marker gene to the level of expression of said standard control gene, whereby said ratio being approximately equal to the corresponding ratio set forth in Table 1A or Table 2 predicts development of MS within two years of being initially diagnosed with CIS.

### Embodiment 19

The method of any preceding Embodiment, wherein the elevated level of expression of said marker gene or the lowered level of expression of said marker gene is determined by a threshold expression level resulting from a statistical model.

### Embodiment 20

The method of Embodiment 19, wherein said statistical model is obtained using a classifier algorithm selected from a compound covariate predictor, a diagonal linear discriminant analysis, and a support vector machine.

### Embodiment 21

The method of any preceding Embodiment, wherein said patient at high risk of developing MS is a patient with CIS that will develop MS within two years of being initially diagnosed with CIS.

### Embodiment 22

A kit for use in identifying a patient with clinically isolated syndrome (CIS) at high risk of developing multiple sclerosis (MS), said kit comprising;
(i) a nucleic acid comprising a sequence having at least 90%, 91%, 92%, 93%, 94%, 95%, 96%, 97%, 98%, 99%, or 100% identity over at least a 10 nucleotide continuous region with one or more nucleic acids having SEQ ID NO:1 to SEQ ID NO:1021, or a nucleic acid complimentary thereto; and
(ii) an electronic device or computer software capable of comparing a marker gene expression level from said patient to a standard control thereby indicating whether said patient is at high risk of developing MS.

### Embodiment 22A

A kit for use in identifying a patient with clinically isolated syndrome (CIS) at high risk of developing multiple sclerosis (MS), said kit comprising;
(i) a nucleic acid comprising a sequence having at least 90%, 91%, 92%, 93%, 94%, 95%, 96%, 97%, 98%, 99%, or 100% identity over at least a 10 nucleotide continuous region with one or more nucleic acids having SEQ ID NO:1 to SEQ ID NO:1021, or a nucleic acid complimentary thereto.

### Embodiment 23

The kit of Embodiment 22 or 22A, wherein the nucleic acid is at least 10 nucleotides in length.

### Embodiment 24

The kit of Embodiment 22, 22A or Embodiment 23, which comprises a nucleic acid comprising a sequence having at least 90%, 91%, 92%, 93%, 94%, 95%, 96%, 97%, 98%, 99%, or 100% identity over at least a 10 nucleotide continuous region with one or more marker genes wherein said marker gene is selected from ZNF12 (SEQ ID NO:83), C 17orf65 (SEQ ID NO:977), BAT1 (SEQ ID NO:981), ARHGDIA (SEQ ID NO:1000), NAPA (SEQ ID NO:995), ATP5G2 (SEQ ID NO:996), DDX52 (SEQ ID NO:292), NDFIP1 (SEQ ID NO:2), SDAD1 (SEQ ID NO:116), USP7 (SEQ ID NO:1014), MEF2A (SEQ ID NO:1007), AGER (SEQ ID NO:998), RAB1B (SEQ ID NO:1011), GDI1 (SEQ ID NO:986) and BANF1(SEQ ID NO:999).

### Embodiment 25

The kit of Embodiment 22, 22A or Embodiment 23, which comprises a nucleic acid comprising a sequence having at least 90%, 91%, 92%, 93%, 94%, 95%, 96%, 97%, 98%, 99%, or 100% identity over at least a 10 nucleotide continuous region with one or more marker genes wherein said marker gene is selected from ZNF12 (SEQ ID NO:83), C17orf65 (SEQ ID NO:977), BAT1 (SEQ ID NO:981), ARHGDIA (SEQ ID NO:1000), NAPA (SEQ ID NO:995), ATP5G2 (SEQ ID NO:996), DDX52 (SEQ ID NO:292), NDFIP1 (SEQ ID NO:2) and SDAD1 (SEQ ID NO:116).

### Embodiment 26

The kit of Embodiment 22, 22A or Embodiment 23, which comprises a nucleic acid comprising a sequence having at least 90%, 91%, 92%, 93%, 94%, 95%, 96%, 97%, 98%, 99%, or 100% identity over at least a 10 nucleotide continuous region with one or more marker genes wherein said marker gene is selected from USP7 (SEQ ID NO:1014), MEF2A (SEQ ID NO:1007), AGER (SEQ ID NO:998), RAB1B (SEQ ID NO:1011), GDI1 (SEQ ID NO:986) and BANF1 (SEQ ID NO:999).

### Embodiment 27

Use, in the identification of a patient with clinically isolated syndrome (CIS) at high risk of developing multiple sclerosis (MS), of a microarray comprising a nucleic acid immobilised on a solid substrate, said nucleic acid having a sequence having at least 90%, 91%, 92%, 93%, 94%, 95%, 96%, 97%, 98%, 99%, or 100% identity over at least a 10 nucleotide continuous region with one or more marker genes wherein said marker gene is selected from the group consisting of SEQ ID NO:1 to SEQ ID NO:1021.

### Embodiment 28

The use of Embodiment 27, wherein said nucleic acid comprises a sequence having at least 90%, 91%, 92%, 93%, 94%, 95%, 96%, 97%, 98%, 99%, or 100% identity over at least a 10 nucleotide continuous region with one or more marker genes wherein said marker gene is selected from ZNF12 (SEQ ID NO:83), C17orf65 (SEQ ID NO:977), BAT1 (SEQ ID NO:981), ARHGDIA (SEQ ID NO:1000), NAPA (SEQ ID NO:995), ATP5G2 (SEQ ID NO:996), DDX52 (SEQ ID NO:292), NDFIP1 (SEQ ID NO:2) or SDAD1 (SEQ ID NO:116), USP7 (SEQ ID NO:1014), MEF2A (SEQ ID NO:1007), AGER (SEQ ID NO:998), RAB1B (SEQ ID NO:1011), GDI1 (SEQ ID NO:986) and BANF1 (SEQ ID NO:999).

### Embodiment 29

The use of Embodiment 27, wherein said marker gene is ZNF12 (SEQ ID NO:83), C17orf65 (SEQ ID NO:977), BAT1 (SEQ ID NO:981), ARHGDIA (SEQ ID NO:1000), NAPA (SEQ ID NO:995), ATP5G2 (SEQ ID NO:996), DDX52 (SEQ ID NO:292), NDFIP1 (SEQ ID NO:2) or SDAD1 (SEQ ID NO:116).

### Embodiment 30

The use of Embodiment 27, wherein said marker gene is USP7 (SEQ ID NO:1014), MEF2A (SEQ ID NO:1007), AGER (SEQ ID NO:998), RAB1B (SEQ ID NO:1011), GDI1 (SEQ ID NO:986) or BANF1 (SEQ ID NO:999).

### Embodiment 31

The use of Embodiment 27, wherein a plurality of nucleic acids are immobilised on said solid substrate, said plurality of nucleic acids having a sequence having at least 90%, 91%, 92%, 93%, 94%, 95%, 96%, 97%, 98%, 99%, or 100% identity over at least a 10 nucleotide continuous region with all marker gene sequences listed in Table 18, Table 19, Table 1A, Table 2, Table 4, Table 8, or Table 13.

### III. Examples

### 1. Molecular Signature in CD4+ Cells Segregates Cis Patients from Controls

Gene expression microarray analysis was performed in negatively isolated naïve CD4+ T-cells obtained from 37 CIS patients after initial clinical presentation (mean 4.5+/−2.6 months) and from 29 controls matched for age and gender. Four arrays failed to pass our quality control protocol and were thus excluded from further analysis. Demographic characteristics of the remaining patients (n=34) and controls (n=28) were similar (Table 3). Analysis was focused on the 1,718 probe sets that showed at least a 2 fold-change from each gene's median value in more than 20% of the samples.

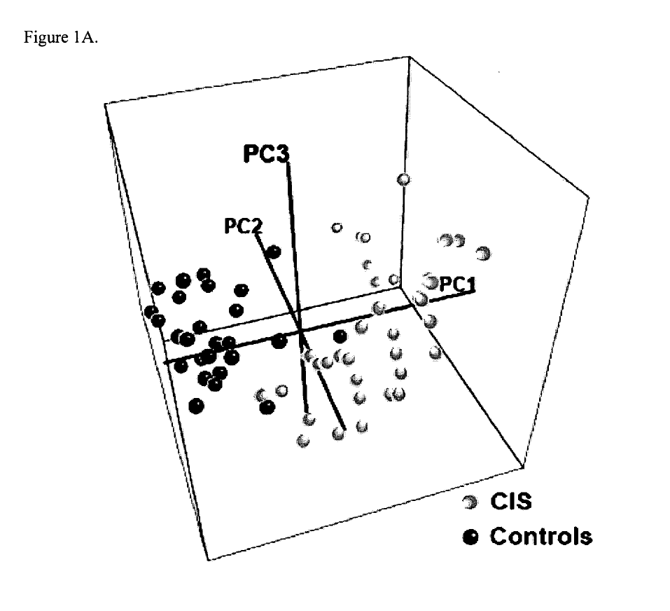

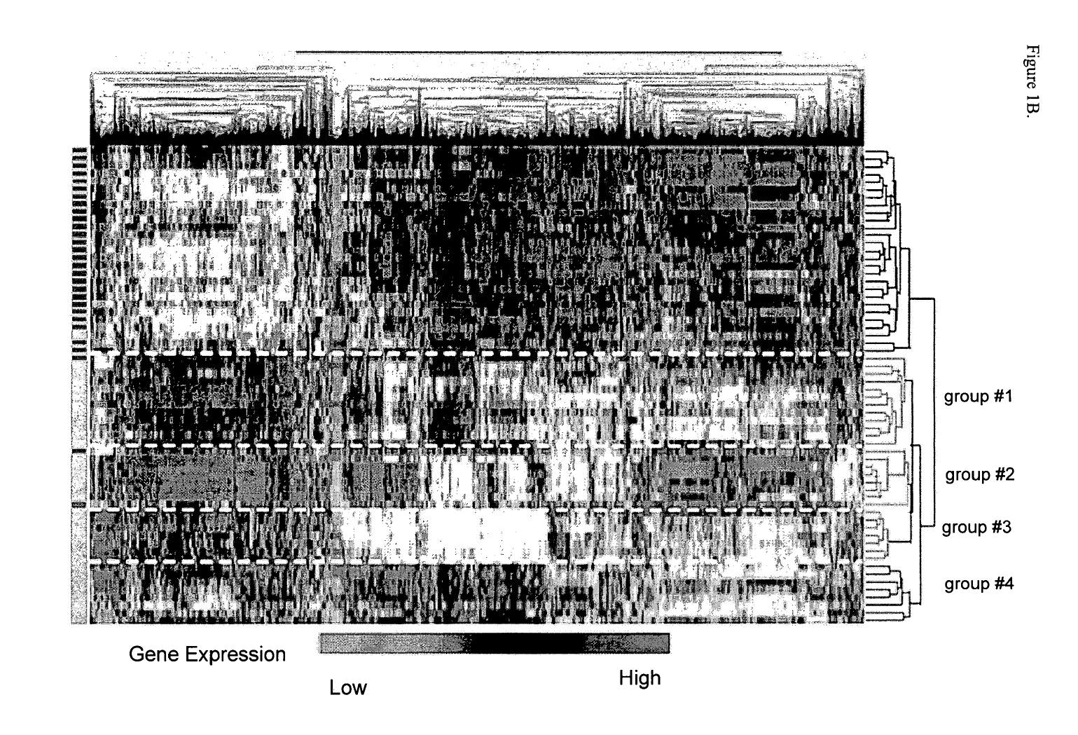

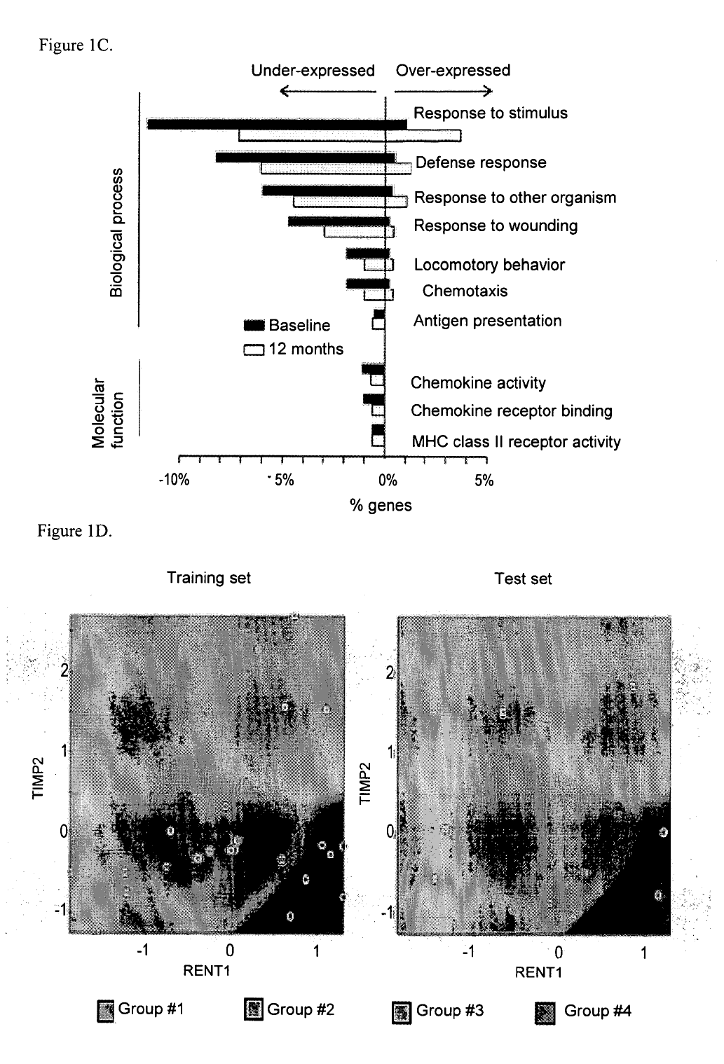

Principal component analysis (PCA) using expression values from these 1,718 probe sets showed a clear segregation between controls and CIS samples (FIG. 1a). Furthermore, a hierarchical clustering of all samples based on the expression of the same probe sets discriminated CIS from controls with high accuracy (only 3 samples were misclassified, 2 controls and 1 patient) (FIG. 1b). The robustness index for this classification was 99.8% (see material and methods). Noteworthy, PCA and hierarchical clustering were performed with the 1,718 probe sets with the highest variance across all samples, but without any prior statistical testing for differences between cases and controls.

When subjected to a T-test, 975 probe sets were found differentially expressed between CIS and controls after correction for multiple comparisons (FDR<0.1 (Table 4). Interestingly, most of the discriminating transcripts (70%) were under-expressed in CIS whereas the remaining 30% were over-expressed. This finding is in agreement with previous observations that downregulated genes greatly outnumber upregulated genes in T lymphocytes from MS patients when studied by gene expression microarrays (6, 7) or by FACS (8).

Gene ontology (GO) enrichment of these 975 differentially expressed genes revealed alteration of major molecular functions and biological processes (FIG. 1c). Unexpectedly for an autoimmune disease, most genes involved in inflammatory responses were downregulated in CIS individuals including pro-inflammatory cytokines, chemokines, integrins, and HLA class II molecules (Table 4). One possible explanation for overall reduced transcript abundance is cell death. However, there was no evidence found that this generalized downregulation was due to increased apoptosis. Quite the opposite, transcripts coding for the anti-apoptotic molecule BAX were increased by 2-fold whereas those coding for the pro-apoptotic molecules BCL-2 and cytochrome C (CYCS) were decreased by 2-fold. Over-expressed genes were mostly involved in protein metabolism (27% of over-expressed genes) and/or nucleotide binding (23% of over-expressed genes). Altogether, these results suggest a decreased inflammatory activity in CD4+ T cells from CIS patients.

On the basis of transcriptional activity, samples from CIS patients segregated into 4 groups (groups #1, 2, 3, 4) corresponding to the first 4 splits of the dendrogram (Robustness index 99.4%) (FIG. 1b). Furthermore, the likelihood that this segregation occurred by chance using IBIS, a supervised machine learning approach was investigated (9) In short, the accuracy of 2-gene Bayesian models created was measured by using only a “training” set (70% samples) to classify a left out “test” set (30% samples) into the 4 previously defined CIS groups (FIG. 1d). The mean accuracy after 10 randomized splits of the samples for the top 7 gene pairs was more than 80% (Table 5).

### 2. Clinical and Radiological Characteristics of the 4 CIS Groups

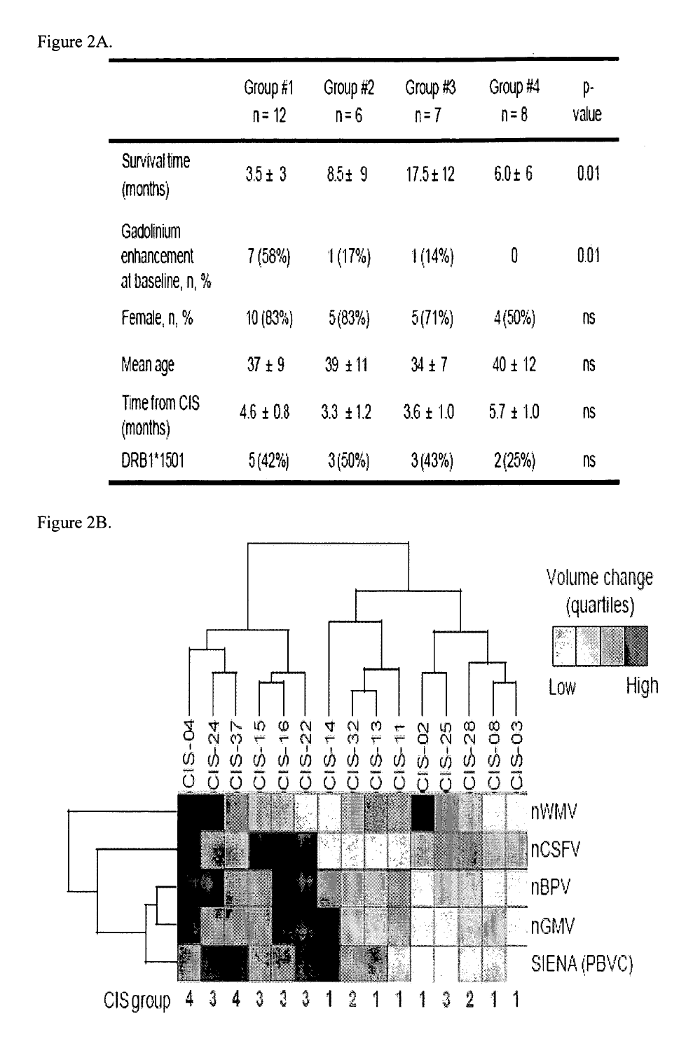

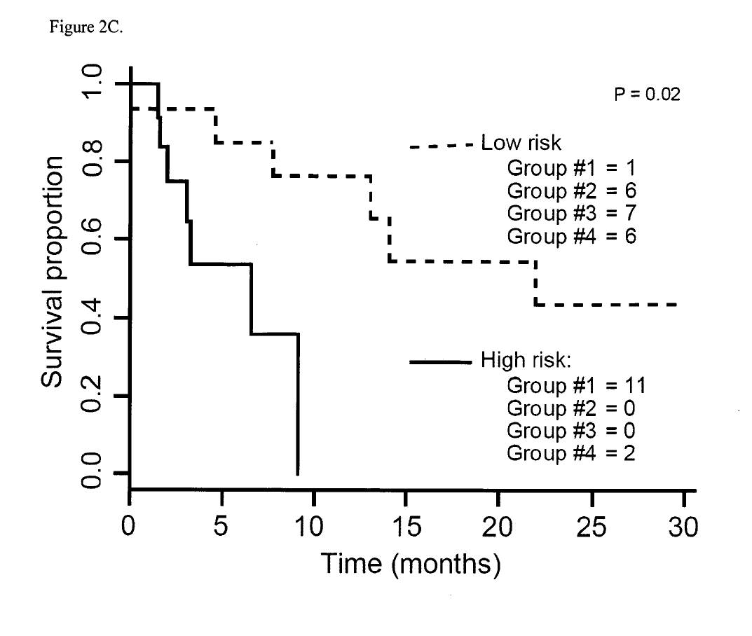

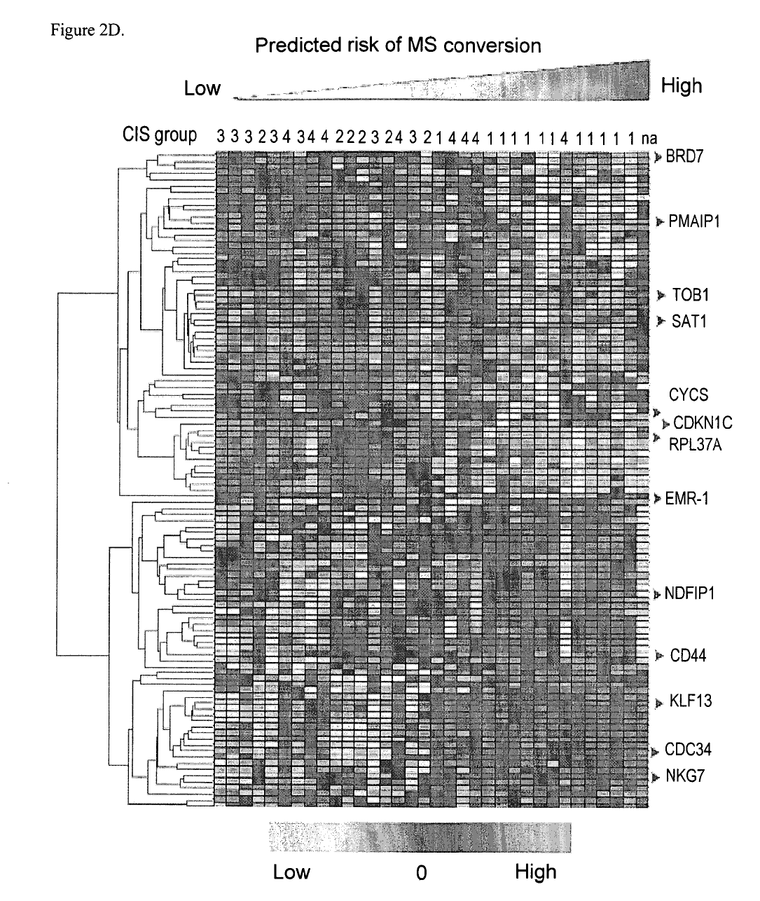

Patients from each of the 4 CIS transcriptional groups did not differ significantly according to age, gender, ethnic background, time from initial clinical event, or HLA-DRB1*1501 status (FIG. 2a). In contrast, time to conversion into CDMS was significantly shorter in patients from group #1 (FIG. 2a). The proportional Cox-regression hazard ratio for patients in group #1 was 3.5 (95% confidence interval [1.4-8.8], p=0.008) indicating a much higher risk of MS conversion for these individuals. Gadolinium enhancement on brain MRI shortly after clinical presentation was significantly higher in patients from group #1 compared to those from other groups, also indicating a higher disease activity (FIG. 2a). However, only 58% of the patients in group #1 showed gadolinium enhancement within 3 months of diagnosis.

In order to evaluate the concordance of gene expression with neurodegeneration, it was investigated to what extent changes in brain volume differed across the four CIS groups. To avoid biases related to therapy, only the 15 patients who did not receive disease-modifying therapy during the first year after diagnosis were included in this analysis. Normalized brain parenchyma (nBPV), white matter (nWMV), grey matter (nGMV) and CSF (nCSFV) volumes were not significantly different among the 4 groups at baseline. However, hierarchical clustering of changes after 1 year in these quantitative MRI parameters segregated patients in two major groups (FIG. 2b). One group displayed higher volume change (i.e. increase in CSF and decrease in brain volume), indicating a larger degree of neurodegeneration. The other group was characterized by relatively low change in CSF or brain volume. Interestingly, all group #1 patients were clustered into the latter group (Chi-square test, p=0.01).

To further explore what information about conversion to MS is contained in gene expression at the CIS stage, a predictive model of survival (i.e. conversion to MS) was built based on supervised principal components (10). The resulting model contained mRNA gene products hybridizing to 28 probe sets set forth in Table 2 and allowed a segregation of CIS into high and low risk groups (FIG. 2c). The separation remained significant even after adjustment for age, gender, and HLA-DRB1*1501 status (data not shown). Patients segregated into the high risk group based on their gene expression (red line), converted to MS by 9 months of follow up. Remarkably, 11 out of the 12 patients from group #1 were clustered into the high-risk group, thus resulting in a sensitivity of 92% and a specificity of 86%. This confirms the previous observation that gene expression-based segregation of group #1 patients is associated with high risk of MS conversion.

### 3. Gene Expression Still Differentiates Group #1 from Other CIS Patients a Year Later

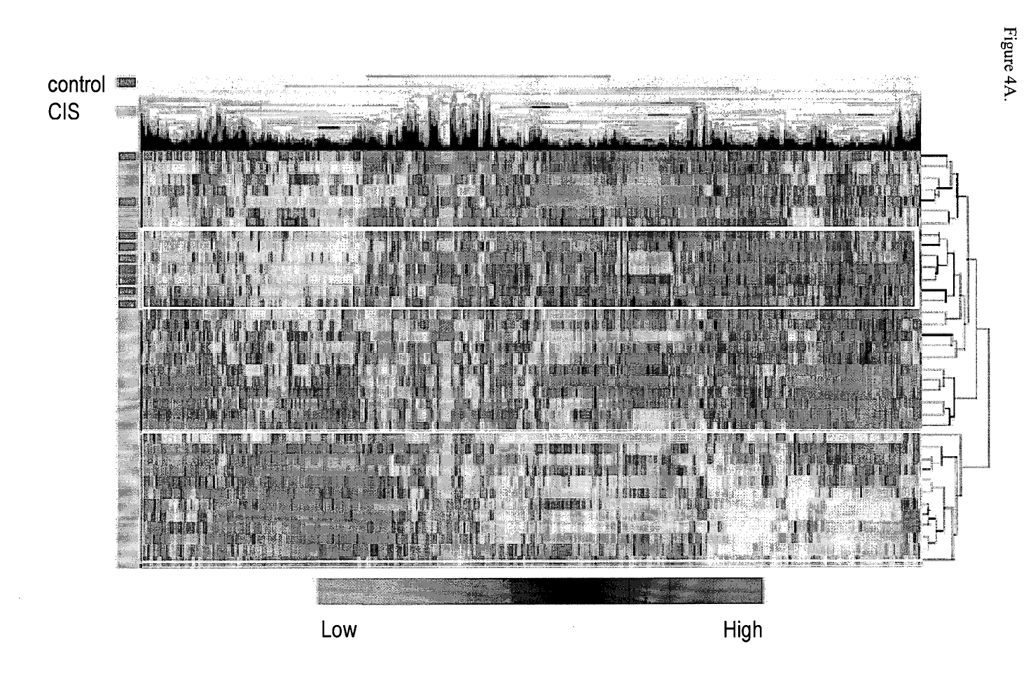

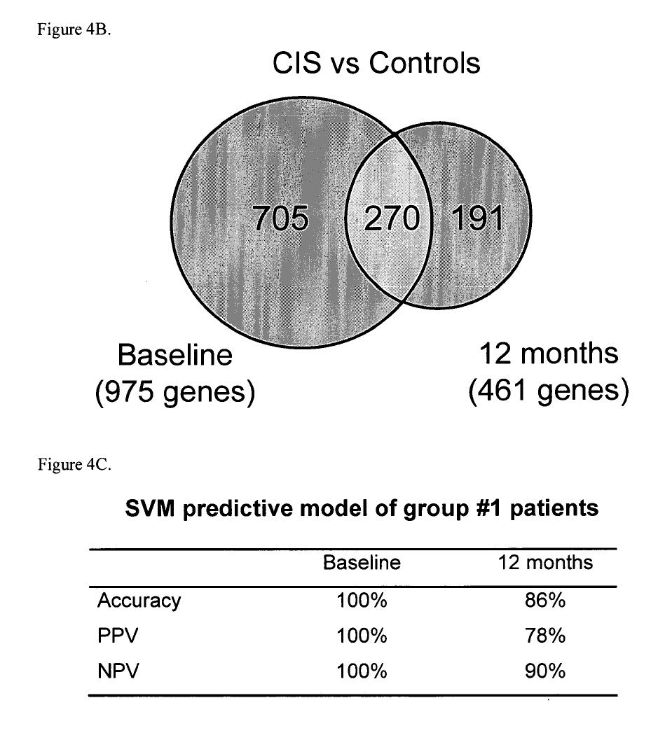

Differential gene expression observed shortly after CIS diagnosis may either reflect an acute and transient biological response to the disease and/or a predisposing causative signature. To investigate this further and to confirm our observations, samples from the same individuals where collected and processed one year (mean 11+/−3 months) after diagnosis of CIS. For this follow-up, 31 CIS and 9 controls were available. Hierarchical clustering of these samples performed with the same 1,718 genes identified at baseline still discriminated CIS from controls (FIG. 4a). Among them, differential expression in 461 transcripts was statistically significant, including 270 (59%) of the 975 genes originally found to be differentially expressed at baseline (FIG. 4b). Remarkably, the first split of the samples' dendrogram segregated 100% of the previously identified group #1 patients from the rest of CIS patients and controls (Yellow box, FIG. 4a). In contrast, previously identified CIS groups #2, #3 and #4 were no longer detected. In order to measure the robustness of group #1 signature along time, a support vector machine (SVM) classifier (with 10-fold leave-one-out crossvalidation) with the 108 expression values obtained at baseline (training set) was built and used to predict the status of samples obtained at 12 months (test set). This model classified group #1 samples at baseline with 100% accuracy, positive predictive value (PPV), and negative predictive value (NPV) (FIG. 4c). After 12 months, the same model was able to predict group #1 patients with an accuracy of 86%, a PPV of 78% and a NPV of 90% (FIG. 4c). Altogether these results suggest that the molecular signature found in naïve CD4+ T cells of group #1 CIS patients is stable for at least 1 year.

### 4. TOB1 Abrogates T Cell Quiescence

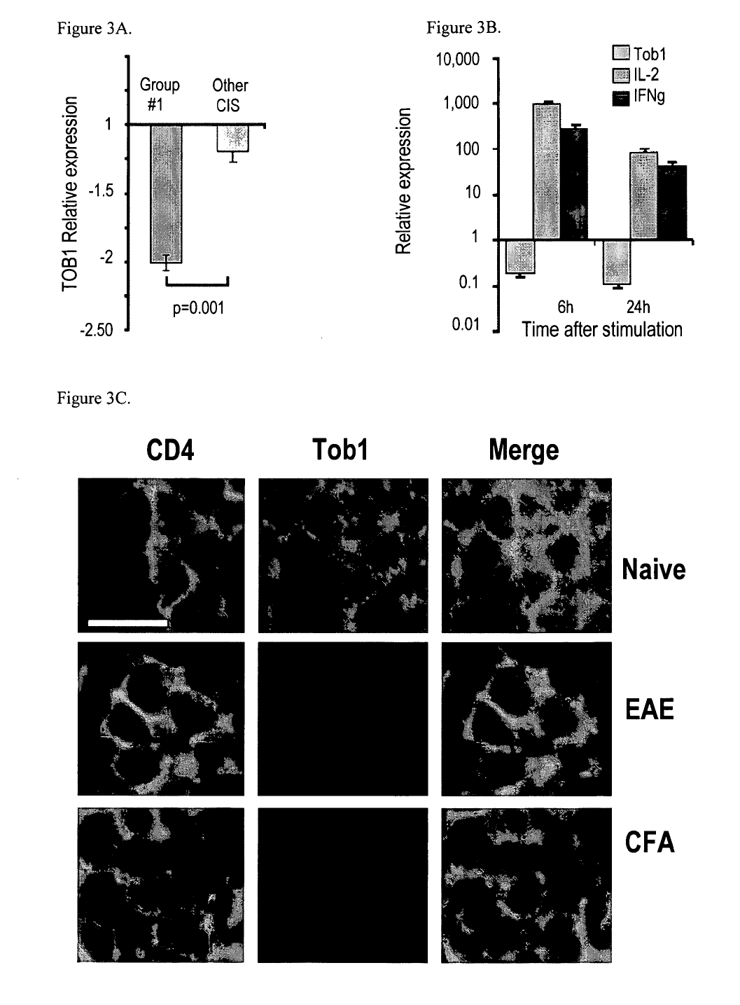

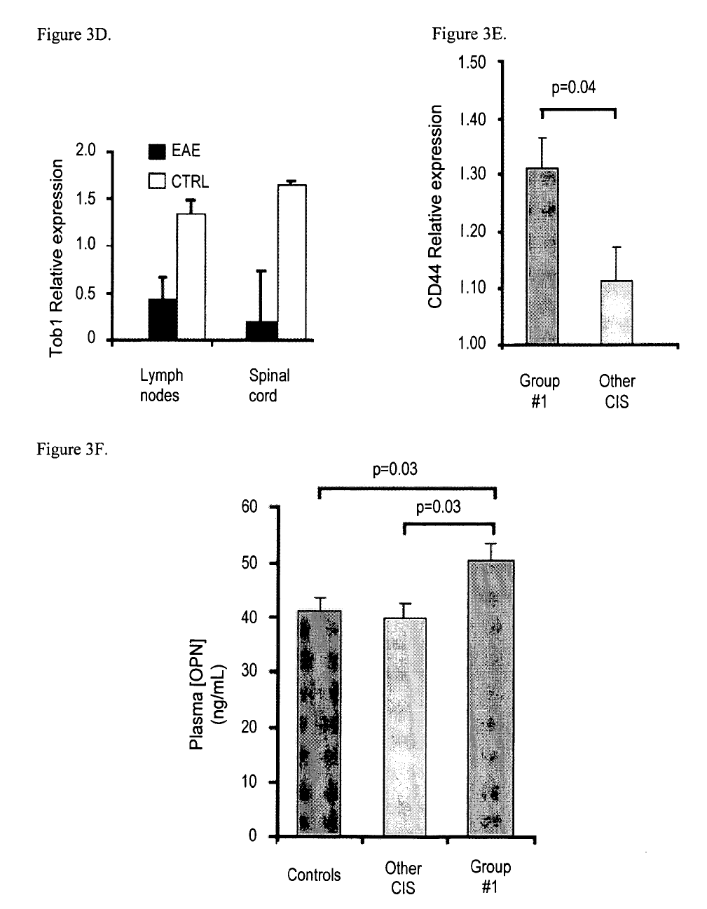

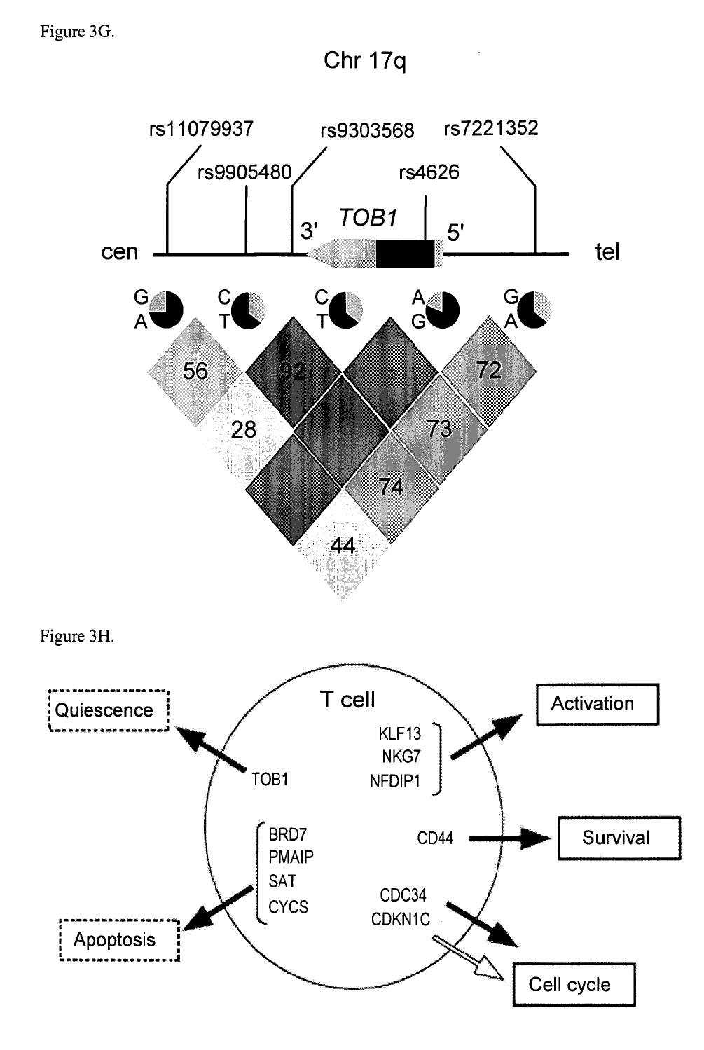

Because group #1 signature persists along time, focus lay on this group which also appears to constitute a relatively consistent biological entity. Among the RNA gene products hybridizing to the 975 probe sets set forth in Table 4 whose expression differentiates CIS from controls at baseline, RNA gene products hybridizing to 108 probe sets set forth in Table 1A were also differentially expressed between group #1 and the other CIS groups combined (FIG. 2d, Table 6). With the exception of a ribosomal protein (RPL37A), the most under-expressed gene in group#1 patients was TOB1 (transducer of ERBB-2, 1) a finding confirmed by RT-PCR (FIG. 3a). TOB1 is a member of the APRO (anti-proliferative) family and has been shown to repress T cell proliferation (11). A strong downregulation of TOB1 upon in-vitro activation of peripheral-blood CD4+ T cells from control individuals (n=3, FIG. 3b) was observed, which is in accordance with previous studies (12). To examine whether downregulation of TOB1 is associated with T cell proliferation in-vivo, C57/B16 mice were immunized with either MOG₃₅₋₅₅ or CFA and investigated TOB1 protein levels in the lymph nodes by immunofluorescence. In agreement with the molecular and in-vitro data, TOB1 immunostaining was decreased in both groups 3 days after immunization while higher levels of the protein were detected in the lymph nodes of naïve mice (FIG. 3c). These results suggest that TOB1 downregulation can be detected as T cells proliferate in response to either a specific (MOG peptide) or unspecific (CFA) antigenic stimulus.

C57/B16 mice injected with MOG₃₅₋₅₅ reproducibly develop experimental autoimmune encephalomyelitis (EAE), a widely-employed laboratory model for MS. In contrast to the sustained inflammation endured by animals with EAE, the response to CFA is expected to be transient and it was hypothesized that patterns of Tob1 expression should reflect this difference. To test this hypothesis, the database from two previous experiments was searched, in which high throughput gene expression in lymph nodes and spinal cords at the peak of EAE was measured (13, 14). As expected, Tob1mRNA expression was decreased in both lymph nodes and spinal cords of EAE animals compared to CFA controls (FIG. 3d).

In addition to intra-molecular mechanisms, engagement of transmembrane receptors may contribute in regulating T cell homeostasis. Among the 3 differentially expressed genes coding for transmembrane receptors (SIGLEC10, EMR1 and CD44) only CD44, was over-expressed in CIS patients (Table 6). This upregulation was confirmed by qRT-PCR (FIG. 3e). Of interest, CD44 serves as a receptor for osteopontin (OPN or SPP1), a pleiotropic molecule that has been shown to be highly expressed in both MS plaques and EAE lesions (15). OPN acts as a key promoter of disease severity in EAE by directing the differentiation and survival of Thl cells (16). Plasma OPN levels in CIS group #1 patients were significantly higher than both other CIS and controls (FIG. 3f).

Since CIS patients classified as group #1 converted to CDMS earlier than other CIS patients, it was hypothesized that TORI is also implicated in the progression of disease once established. A genetic effect would be then expected in CDMS patients showing extreme phenotypes (mild or severe). This hypotheses was tested by genotyping 5 SNPs located within or near the gene (FIG. 4g) in individuals selected from a cohort of more than 1,200 RRMS patients that were clinically classified as either “mild” (EDSS<3 15 years after onset, n=62) or “severe” (EDSS>6 10 years after onset, n=74). Allelic frequencies were analyzed by logistic regression and case-control association. Differences in allelic frequencies for marker rs4626 (coding, synonymous) between mild and severe cases were statistically significant by logistic regression for both genotype and trend tests (marker rs7221352 also showed both effects although without reaching statistical significance). The same two markers showed statistical significance in the allele case-control (mild vs. severe) analysis (Table 7). Haplotype analysis with these two markers also showed statistical significance when the associated, but not the neutral alleles were considered (G-A, exact p-value=0.0115; A-G, exact p-value=0.0353). Altogether this data suggests that while TORI downregulation identifies CIS patients at higher risk of conversion to CDMS, there is also a genetic association between markers in this gene and the clinical progression of CDMS patients.

### 5. Patients and Samples

The study cohort consisted of 37 untreated CIS patients and 29 healthy control subjects matched for age and sex, evaluated at the UCSF Multiple Sclerosis Center. CIS patients were identified as subjects presenting with a first well-defined, neurological event persisting for more than 48 hours involving the optic nerve, brain parenchyma, brainstem, cerebellum, or spinal cord. All CIS patients demonstrated at least two abnormalities on brain MRI measuring greater than 3 mm². Patients were followed for an average of 20 (+/−8) months. Time to conversion was defined as the delay between recruitment and next clinical event or the date of identified MRI changes fulfilling the McDonald criteria (5). Written informed consent was obtained from all study participants.

### 6. Magnetic Resonance Imaging

MRI scans for all subjects were acquired on a 1.5 T GE (GE) MRI scanner with a standard head coil. All CIS subjects were scanned every 3 months during the first year of follow-up and then every 6 months during the second year. T2 hyperintense lesions were identified on simultaneously viewed T2 and proton density-weighted dual echo (1 mm×1 mm×3 mm pixels, interleaved slices, 20 ms and 80 ms echo times) images with regions of interest drawn based on a semi-automated threshold with manual editing as described elsewhere (26) Annual percent brain volume change (PBVC) was calculated from high resolution 3D T1-weighted spoiled gradient recalled echo volumes (pixel size of 1 mm×1 mm×1.5 mm, 124 slices, flip angle 40°) using SIENA (27).

### 7. RNA Preparation and Hybridization

Blood samples were collected at the time of recruitment into the study (baseline) and after 12 months. Peripheral blood mononuclear cells (PBMC) were separated on a Ficoll gradient and frozen in liquid nitrogen until needed. Naïve CD4+ T cells were isolated by negative selection using Dynabeads® (Invitrogen). CD4+ T cells purity was assessed by FACS (>95%, data not shown). RNA was then extracted using RNeasy® Mini kit (Quiagen), amplified with MessageAmp™ II a RNA kit (Ambion) and labeled with Bio-11-UTP for subsequent hybridization onto Affymetrix® Human Genome U133 Plus2.0 arrays (TGEN). Thus, the probe set identifier numbers set forth in the Tables below (including Tablesl8, 19, 1A, 2, 4, 8 and 13) are in reference to Affymetrix® Human Genome U133 Plus2.0 arrays.

### 8. Statistical Analysis

Quality control (QC) analysis of the arrays was performed using the Bioconductor package, available at the bioconductor.org website. In order to pass QC, arrays had to have at least 40% of their probe sets called present and had to have similar RNA degradation slopes, GAPDH and beta-actin ratios, scaling factors, histograms and box plot of intensities. Arrays were normalized using RMA (28). Statistical analyses were carried out using BRB-array Tools (Biometrics Research Branch, NIH). For multiple comparison correction, genes were considered differentially expressed if the univariate p-value was less than 0.001 and False discovery rate (FDR) less than 0.1 (29). Genes predicting MS conversion were determined using the Survival Analysis Prediction Tool of BRB-array Tools. The 2 survival risk groups were built using PCA with a p-value set at 0.001 for univarietely correlated genes with survival and leave-one-out-cross validation. (10) For cases with above and below average risk (50^{th} percentile) Kaplan-Meier survival curves were used. Hierarchical clustering was performed using Genes@work® software (IBM Research). To gauge robustness in the classification, the dataset was perturbed by adding random (white) Gaussian noise using the median variance of the dataset and re-clustered the samples 100 times. The index of robustness is the mean percentage of times a pair of samples remained in the same cluster. To investigate the likelihood that segregation into 4 groups occurred by chance, the Integrated Bayesian Inference System (IBIS) was used, which is a supervised machine learning approach (9).

### 9. Univariate and Multivariate Statistical Models

In order to calculate the marker to GAPDH ratio in a patient, univariate and multivariate statistical models are used. In univariate statistical models, the characteristic of each individual gene in classifying samples as being high or low risk genes is determined. In multivariate models, the best possible combination of two or more genes that can maximize the positive predictive value (PPV) or negative predictive value (NPV) is established. The positive predictive value, is defined as the number of true positives per total of true and false positives, whereas the negative predictive value describes the number of true negatives per total of true negatives and false negatives. Applying this statistical model provides methods to discriminate between high risk and low risk patients.

As discussed above, 108 probe sets were identified (Table 1A) by T-test analysis that hybridized to gene products that were differentially expressed between group#1 and other subjects. And 28 probe sets were identified (Table 2) by principal-component-based survival analysis that hybridized to gene products that were differentially expressed by high risk CIS patients. The combined set of 136 probe sets (108+28) were used to search for classifiers that could discriminate between the two groups with a reduced number of genes. Using compound covariate predictor (CCP), diagonal linear discriminant analysis (DLDA), and support vector machines (SVM) classifiers (see below), 13 probe sets were identified (Table 8) that hybridized to gene products that were differentially expressed. The CCP, DLDA and SVM were run with default parameters and within the BRB array tools application available from the National Cancer Institute. For each classifier a specific weight was assigned to each probe set as set forth in Table 9. The expression value of each probe set was normalized by that of two housekeeping (HK) genes: GAPDH and ACTB, with the results are provided in Tables 10A to 12C, which detail the predictive value of the statistical model by providing the number of CIS patients that developed MS within nine months (MS) and the number that did not develop MS within nine months (No MS) and the corresponding prediction based on the threshold value.

An independent (network-based) search was conducted based on the hypothesis that groups of genes whose products interact physically are likely to define biologically functional modules, as described in Ideker, T., et al. (2002). “Discovering regulatory and signalling circuits in molecular interaction networks.” Bioinformatics 18 Suppl 1: S233-40. Unlike with classical statistical analyses, identification of these modules allows for direct biological interpretation of the results. Briefly, we implemented a sub-network identification tool based on the algorithm previously described by Ideker et al. to identify groups of functionally related genes that could classify high versus low risk CIS patients. This algorithm consists of the following steps. First, a protein interaction database was downloaded locally. Second, starting from each node in the network, a sub-network was recursively grown by the addition of one neighboring node at a time. At each step, a scoring function was computed based on the mutual information between the weighted average of the expression values of all nodes considered at this step, and the vector of phenotypes (case versus control, high vs. low risk, etc). Third, the sub-network continued to grow until addition of a new node did not increase the score significantly. Three classifiers were constructed using the CCP, DLDA, and SVM algorithms. The network based search resulted in the identification of 6 probe sets (Table 13) that hybridized to gene products that were differentially expressed. For each classifier a specific weight was assigned to each probe set as set forth in Table 14. The expression value of each probe set was normalized by that of two housekeeping (HK) genes: GAPDH and ACTB. The predictive value and threshold values for the 6 probe sets were calculated, with the results provided in Table 15A to 17C, which detail the predictive value of the statistical model by providing the number of CIS patients that developed MS within nine months (MS) and the number that did not develop MS within nine months (No MS) and the corresponding prediction based on the threshold value.

The compound covariate predictor (CCP) used in the above studies is a weighted linear combination of log-ratios (or log intensities for single-channel experiments) for genes that are univariately significant at the specified level. By specifying a more stringent significance level, fewer genes are included in the multivariate predictor. Genes in which larger values of the log-ratio pre-dispose to class 2 rather than class 1 have weights of one sign, whereas genes in which larger values of the log-ratios pre-dispose to class 1 rather than class 2 have weights of the opposite sign. The univariate t-statistics for comparing the classes are used as the weights. The CCP is described in further detail in Radmacher M D, McShane L M, and Simon R. A paradigm for class prediction using gene expression profiles. Journal of Computational Biology 9:505-511, 2002; and I Hedenfalk, D Duggan, Y Chen, M Radmacher, M Bittner, R Simon, P Meltzer, B Gusterson, M Esteller, M Raffeld, et al. Gene expression profiles of hereditary breast cancer, New England Journal of Medicine 344:539-548, 2001.

The Diagonal Linear Discriminant Analysis (DLDA) used in the above studies is similar to the Compound Covariate Predictor, but not identical. It is a version of linear discriminant analysis that ignores correlations among the genes in order to avoid over-fitting the data. Many complex methods have too many parameters for the amount of data available. Consequently they appear to fit the training data used to estimate the parameters of the model, but they have poor prediction performance for independent data. The DLDA is described in further detail in McLachlan G J. Discriminant Analysis and Statistical Pattern Recognition Wiley-Interscience; New Ed edition (Aug. 4, 2004); and Dudoit S, Fridlyand J, Speed T P. Comparison of discrimination methods for the classification of tumors using gene expression data, Journal of the American Statistical Association 97:77-87, 2002).

The support vector machine (SVM) used in the above studies is a class prediction algorithm that has appeared effective in other contexts and is currently of great interest to the machine learning community. The SVM predictor can employ a variety of functions, as known in the art. In some embodiments, the SVM predictor is a linear function of the log-ratios or the log-intensities that best separates the data subject to penalty costs on the number of specimens misclassified. The SVM is described in further detail in Vapnik V. The Nature of Statistical Learning Theory. Springer-Verlag, 1995.

### 10. Quantitative RT-PCR

Master mix was prepared essentially as described previously, (9) with the addition of 200 μM ROX (Sigma), and overlaid on top of each well of a freshly thawed 384-well plate containing 5 ng of RNA in each well. Reactions were performed in triplicates using an ABI 7900 Sequence Detection System (Applied Biosystems).

### 11. Immunofluorescence and ELISA

Draining lymph nodes from either naive or injected (MOG₃₅₋₅₅ or CFA alone) C57/B16 mice were removed, washed in PBS and then embedded in OCT and frozen. Sections were cut at 6 μm on a cryostat and stained for immunofluorescence examination using either a rabbit anti-TOB1 polyclonal antibody (H-70, Santa Cruz Biotechnology Inc. CA), or a purified rat anti CD4 antibody (BD Pharmingen). Secondary antibodies were anti-rabbit Alexa 488 (Molecular Probes, Eugene Oreg.) and anti-rat Alexa 594 (Molecular Probes). ELISAs for OPN were carried out using the Quantikine kit (R&D Systems) according to manufacturer's instructions.

### 12. Genotyping of TOB1 SNPs

Five single nucleotide polymorphisms (SNP) located within or near TOB1 were selected for genotyping in 62 mild and 74 severe MS patients. Mild disease was defined as EDSS<3 after 15 years of onset while severe was defined as EDSS>6 after 10 years of onset. Genotyping assays were carried out in 384-well plates using TaqMan® Universal PCR Master Mix on an ABI GeneAmp PCR System 7900 (Applied Biosystems). Statistical tests were carried out in SAS and Jmp Genomics suite (SAS). For haplotype analysis, exact p-values were calculated using the EM algorithm in a Monte Carlo approach with 10,000 permutations.

### 13. Tables

Tables 1-19 follow. Tables 1A, 1B and 2 provide differential gene expression analysis data. Table 3 provides data regarding subject characteristics at baseline. Table 4 provides a list of 975 genes differentially expressed between CIS and controls at baseline. Table 5 provides data regarding mean predictive accuracy of the top seven gene pairs. Table 6 provides data relating to the signature of group #1 patients. And Table 7 provides genotyping data of 5 TOB1 SNP in patients with mild (n=62) or severe (n=74) MS. Tables 9 to 19 present data resulting form the statistical model analysis as described herein. Terms used in the tables are as follows: The term “SD” in the context of statistical analysis refers to the standard deviation, as known in the art. The term “Ave.” refers to the statistical average, as known in the art. The term “Grpl” refers to Group #1 as described herein.

### IV. References

1. Hauser S L & Goodin D S (2005) in Harrison's Principles in Internal Medicine, eds. Braunwald E, Fauci A D, Kasper D L, Hauser S L, Longo D L, & Jameson J L (McGraw-Hill, New York), pp. 2461-2471.
2. Kappos L, et al. (2006) Treatment with interferon beta-1b delays conversion to clinically definite and McDonald M S in patients with clinically isolated syndromes. Neurology 67, 1242-1249.
3. Korteweg T, et al. (2006) MRI criteria for dissemination in space in patients with clinically isolated syndromes: a multicentre follow-up study. Lancet Neurol 5, 221-227.
4. Brex P A, et al. (2002) A longitudinal study of abnormalities on MRI and disability from multiple sclerosis. N Engl J Med 346, 158-164.
5. McDonald W I, et al. (2001) Recommended diagnostic criteria for multiple sclerosis: guidelines from the International Panel on the diagnosis of multiple sclerosis. Ann Neurol 50, 121-127.
6. Satoh J, et al. (2005) Microarray analysis identifies an aberrant expression of apoptosis and DNA damage-regulatory genes in multiple sclerosis. Neurobiol Dis 18, 537-550.
7. Satoh J, et al. (2006) T cell gene expression profiling identifies distinct subgroups of Japanese multiple sclerosis patients. J Neuroimmunol 174, 108-118.
8. Kantor A B, et al. (2007) Identification of short-term pharmacodynamic effects of interferon-beta-1a in multiple sclerosis subjects with broad-based phenotypic profiling. J Neuroimmunol 188, 103-116.
9. Baranzini SE, et al. (2005) Transcription-based prediction of response to IFNbeta using supervised computational methods. PLoS Biol 3, e2.
10. Bair E & Tibshirani R (2004) Semi-supervised methods to predict patient survival from gene expression data. PLoS Biol 2, E108.
11. Tzachanis D, et al. (2001) Tob is a negative regulator of activation that is expressed in anergic and quiescent T cells. Nat Immunol 2, 1174-1182.
12. Yusuf I & Fruman D A (2003) Regulation of quiescence in lymphocytes. Trends Immunol 24, 380-386.
13. Baranzini S E, Bernard C C, & Oksenberg J R (2005) Modular transcriptional activity characterizes the initiation and progression of autoimmune encephalomyelitis. J Immunol 174, 7412-7422.
14. Otaegui D, et al. (2007) Increased transcriptional activity of milk-related genes following the active phase of experimental autoimmune encephalomyelitis and multiple sclerosis. J Immunol 179, 4074-4082.
15. Chabas D, et al. (2001) The influence of the proinflammatory cytokine, osteopontin, on autoimmune demyelinating disease. Science 294, 1731-1735.
16. Hur E M, et al. (2007) Osteopontin-induced relapse and progression of autoimmune brain disease through enhanced survival of activated T cells. Nat Immunol 8, 74-83.
17. Tintoré M, et al. (2001) Isolated demyelinating syndromes: comparison of CSF oligoclonal bands and different MR imaging criteria to predict conversion to CDMS. Multiple Sclerosis 7, 359-363.
18. Berger T, et al. (2003) Antimyelin antibodies as a predictor of clinically definite multiple sclerosis after a first demyelinating event. N Engl J Med 349, 139-145.
19. Kuhle J, et al. (2007) Lack of association between antimyelin antibodies and progression to multiple sclerosis. N Engl J Med 356, 371-378.
20. Orban T, et al. (2007) Reduced CD4+T-cell-specific gene expression in human type 1 diabetes mellitus. J Autoimmun 28, 177-187.
21. Tzachanis D, Lafuente E M, Li L, & Boussiotis V A (2004) Intrinsic and extrinsic regulation of T lymphocyte quiescence. Leuk Lymphoma 45, 1959-1967.
22. Matsuoka S, et al. (1995) p57KIP2, a structurally distinct member of the p21CIP1 Cdk inhibitor family, is a candidate tumor suppressor gene. Genes Dev 9, 650-662.
23. Plon S E, Leppig K A, Do H N, & Groudine M (1993) Cloning of the human homolog of the CDC34 cell cycle gene by complementation in yeast. Proc Natl Acad Sci USA 90, 10484-10488.
24. Ousman SS, et al. (2007) Protective and therapeutic role for alphaB-crystallin in autoimmune demyelination. Nature 448, 474-479.
25. Vogt M H, Lopatinskaya L, Smits M, Polman C H, & Nagelkerken L (2003) Elevated osteopontin levels in active relapsing-remitting multiple sclerosis. Ann Neurol 53, 819-822.
26. Blum D, et al. (2002) Dissociating perceptual and conceptual implicit memory in multiple sclerosis patients. Brain Cogn 50, 51-61.
27. Smith S M, De Stefano N, Jenkinson M, & Matthews P M (2001) Normalized accurate measurement of longitudinal brain change. J Comput Assist Tomogr 25, 466-475.
28. Irizarry R A, et al. (2003) Summaries of Affymetrix GeneChip probe level data. Nucleic Acids Res 31, e15.
29. Benjamini Y & Hochberg Y (1995) Controlling the false discovery rate: a practical and powerful approach to multiple testing. J Roy Stat Soc, 289-300.

TABLE 1A

| | SEQ | | | | Ave. | SD | Ave. |
|---|---|---|---|---|---|---|---|
| Probe set | ID | Marker | Ave. Healthy | | Other | Other | Grp1 |
| Identifier | NO: | Gene | Control | SD Control | CIS | CIS | CIS |
| 217800_s_at | 2 | NDFIP1 | 96.7104533 | 95.75572795 | 254.1 | 321.0 | 713.3 |
| 225247_at | 298 | C19orf6 | 912.8038675 | 351.313034 | 569.7 | 511.6 | 1129.8 |
| 213915_at | 782 | NKG7 | 3748.392227 | 1002.109043 | 1319.4 | 1386.3 | 2381.9 |
| 200041_s_at | 981 | BAT1 | 1737.677862 | 498.0604971 | 2149.6 | 717.0 | 5253.8 |
| 211716_x_at | 1000 | ARHGDIA | 503.0587478 | 199.7016131 | 285.1 | 138.0 | 645.9 |
| 233350_s_at | 300 | TEX264 | 268.0886963 | 55.50046736 | 166.9 | 114.4 | 318.7 |
| 219529_at | 767 | CLIC3 | 110.284748 | 62.05526861 | 35.3 | 46.3 | 65.4 |
| 218607_s_at | 116 | SDAD1 | 143.2487323 | 58.87369449 | 220.0 | 71.4 | 494.5 |
| 202652_at | 290 | APBB1 | 183.2648549 | 49.83218809 | 125.7 | 92.2 | 240.7 |
| 216520_s_at | 138 | TPT1 | 3074.723831 | 3156.269061 | 5236.5 | 2877.9 | 10048.5 |
| 1554021_a_at | 83 | ZNF12 | 30.66688733 | 25.15376602 | 50.1 | 26.5 | 105.6 |
| 204122_at | 900 | TYROBP | 1201.644912 | 376.3319175 | 297.3 | 276.5 | 631.7 |
| 219878_s_at | 504 | KLF13 | 234.6876124 | 59.46239858 | 103.4 | 69.4 | 186.2 |
| 216231_s_at | 242 | B2M | 2860.485868 | 2244.297121 | 4205.6 | 2244.5 | 7880.0 |
| 219571_s_at | 106 | ZNF12 | 23.83333559 | 17.59710007 | 40.5 | 21.0 | 78.8 |
| 214450_at | 879 | CTSW | 1945.934778 | 615.1646128 | 598.0 | 533.2 | 1000.7 |
| 209050_s_at | 997 | RALGDS | 1261.540654 | 316.197773 | 581.1 | 336.9 | 1008.0 |
| 207088_s_at | 992 | SLC25A11 | 297.0283211 | 83.80094822 | 133.2 | 91.3 | 219.7 |
| 1558215_s_at | 71 | UBTF | 27.89662742 | 3.518774288 | 48.0 | 21.6 | 94.5 |
| 200612_s_at | 982 | AP2B1 | 138.6467451 | 46.11948903 | 60.8 | 37.4 | 106.1 |
| 227266_s_at | 257 | FYB | 741.2284544 | 471.163392 | 1058.4 | 415.7 | 1915.2 |
| 205480_s_at | 251 | UGP2 | 567.3176781 | 417.2804445 | 873.5 | 456.7 | 1479.2 |
| 201831_s_at | 213 | VDP | 80.079115 | 44.53736077 | 131.7 | 83.7 | 197.4 |
| 232535_at | 102 | unknown | 17.36604383 | 7.047231356 | 30.9 | 18.5 | 50.0 |
| 244042_x_at | 269 | unknown | 19.88215621 | 7.090260074 | 27.2 | 13.4 | 44.9 |
| 233713_at | 88 | SMYD2 | 35.51625337 | 19.31566529 | 70.1 | 26.5 | 121.3 |
| 211947_s_at | 1001 | BAT2D1 | 315.6667419 | 98.165398 | 142.4 | 52.4 | 241.4 |
| 212368_at | 152 | ZNF292 | 208.4753741 | 103.8816828 | 355.0 | 132.7 | 591.8 |
| 217854_s_at | 573 | POLR2E | 530.0863493 | 113.1975464 | 228.7 | 123.5 | 338.5 |
| 217993_s_at | 96 | MAT2B | 792.2890998 | 587.4647981 | 1582.2 | 714.0 | 2428.0 |
| 236562_at | 154 | ZNF439 | 26.29907812 | 19.04974141 | 45.3 | 19.0 | 71.7 |
| 200033_at | 217 | DDX5 | 3216.520811 | 2823.244939 | 5342.3 | 1586.0 | 8366.5 |
| 221895_at | 225 | MOSPD2 | 130.2392647 | 53.59154667 | 207.4 | 113.6 | 300.4 |
| 201998_at | 987 | ST6GAL1 | 373.7283149 | 190.1599525 | 622.2 | 291.5 | 921.2 |
| 209458_x_at | 971 | HBA1 /// | 804.5334775 | 1999.83385 | 50.0 | 11.6 | 99.4 |
| | | HBA2 | | | | | |
| 241838_at | 40 | unknown | 18.36391457 | 7.052024714 | 39.7 | 19.0 | 63.1 |
| 212540_at | 553 | CDC34 | 321.9275865 | 108.6345653 | 154.4 | 90.3 | 205.2 |
| 212926_at | 164 | SMC5L1 | 84.03206343 | 34.70164192 | 148.2 | 60.6 | 212.6 |
| 202283_at | 769 | SERPINF1 | 111.5260189 | 47.52765099 | 39.0 | 16.3 | 55.9 |
| 200735_x_at | 220 | NACA | 4139.886031 | 2666.221608 | 6913.5 | 1798.1 | 10231.1 |
| 207460_at | 617 | GZMM | 273.0278916 | 93.2137522 | 119.0 | 60.6 | 172.0 |
| 211699_x_at | 967 | HBA1 /// | 577.6797877 | 1613.670589 | 60.9 | 11.9 | 109.1 |
| | | HBA2 | | | | | |
| 208635_x_at | 214 | NACA | 3965.432116 | 2515.963282 | 6736.1 | 1672.7 | 9872.6 |
| 209651_at | 664 | TGFB1I1 | 44.654309 | 20.81058608 | 18.2 | 8.3 | 26.3 |
| 213687_s_at | 165 | RPL35A | 4375.141279 | 2306.488539 | 8120.3 | 1748.7 | 11464.2 |
| 208325_s_at | 882 | AKAP13 | 126.3933081 | 65.66546784 | 36.9 | 11.2 | 57.1 |
| 203375_s_at | 988 | TPP2 | 210.7686166 | 172.6152428 | 324.6 | 134.0 | 449.2 |
| 212063_at | 275 | CD44 | 990.7506707 | 659.8373057 | 1473.5 | 437.5 | 2055.2 |
| 235213_at | 76 | ITPKB | 779.1840666 | 419.5630058 | 1628.2 | 546.5 | 2254.6 |
| 242778_at | 869 | LPXN | 143.6867555 | 84.59695649 | 59.5 | 14.7 | 42.4 |
| 226843_s_at | 884 | PAPD5 | 1910.356104 | 604.1972303 | 798.7 | 245.3 | 547.5 |
| 1569599_at | 578 | SAMSN1 | 28.48629113 | 17.16502805 | 17.2 | 7.8 | 11.0 |
| 207111_at | 669 | EMR1 | 320.6728923 | 146.2818122 | 189.0 | 105.6 | 117.8 |
| 242918_at | 969 | NASP | 124.1118889 | 98.43225996 | 14.1 | 4.0 | 8.9 |
| 1553861_at | 976 | TCP11L2 | 79.22827834 | 43.09611716 | 53.2 | 20.4 | 35.9 |
| 1552807_{'}a_at | 765 | SIGLEC10 | 441.6801421 | 231.9664661 | 219.6 | 108.4 | 132.0 |
| 238545_at | 903 | BRD7 | 1116.910287 | 438.1978068 | 484.6 | 267.0 | 276.9 |
| 202381_at | 433 | ADAM9 | 87.37622443 | 53.85995502 | 63.3 | 36.7 | 35.1 |
| 226982_at | 353 | ELL2 | 448.6067163 | 238.3580447 | 357.9 | 288.0 | 199.1 |
| 202083_s_at | 937 | SEC14L1 | 358.4116609 | 132.1558944 | 125.7 | 71.6 | 69.6 |
| 228284_at | 844 | TLE1 | 223.8286885 | 116.1536573 | 101.7 | 47.2 | 58.4 |
| 222670_s_at | 842 | MAFB | 550.2143555 | 376.934896 | 253.1 | 185.0 | 134.5 |
| 243296_at | 396 | PBEF1 | 342.8300274 | 212.2533748 | 249.0 | 103.5 | 151.6 |
| 212840_at | 104 | KIAA0794 | 199.0536987 | 76.5416961 | 534.8 | 154.3 | 303.4 |
| 1564164_at | 11 | FLJ20054 | 64.23388467 | 26.88863142 | 269.5 | 152.2 | 139.3 |
| 204566_at | 990 | PPM1D | 356.2707527 | 120.3061939 | 314.0 | 182.3 | 160.5 |
| 226643_s_at | 184 | LOC134492 | 34.19986774 | 8.400359057 | 91.7 | 45.4 | 46.0 |
| 228049_x_at | 420 | unknown | 443.0962527 | 126.90203 | 326.6 | 127.9 | 167.2 |
| 217602_at | 1010 | PPIA | 30.95187262 | 11.59178375 | 28.2 | 17.8 | 14.4 |
| 215023_s_at | 530 | PEX1 | 64.51736193 | 26.93983789 | 48.4 | 30.2 | 21.4 |
| 1567213_at | 980 | PNN | 215.39985 | 61.15043081 | 567.9 | 244.8 | 296.4 |
| 209160_at | 845 | AKR1C3 | 443.6579502 | 176.6915813 | 214.3 | 164.7 | 121.1 |
| 205789_at | 376 | CD1D | 49.13639343 | 37.70100424 | 40.5 | 34.7 | 16.2 |
| 208750_s_at | 430 | ARF1 | 103.527944 | 48.2379301 | 94.7 | 84.8 | 39.1 |
| 201151_s_at | 983 | MBNL1 | 49.3888033 | 17.65848473 | 159.4 | 115.2 | 69.2 |
| 212649_at | 1003 | unknown | 25.30370743 | 4.159717144 | 66.1 | 32.3 | 28.8 |
| 229733_s_at | 634 | CBX6 | 76.97125046 | 63.26817093 | 47.5 | 35.4 | 19.9 |
| 203603_s_at | 346 | ZFHX1B | 218.4495249 | 85.64500979 | 185.3 | 110.2 | 99.8 |
| 201110_s_at | 858 | THBS1 | 62.01388044 | 51.89044949 | 29.1 | 23.0 | 11.1 |
| 1555226_s_at | 554 | C1orf43 | 89.84621232 | 47.08814838 | 73.1 | 64.2 | 26.8 |
| 1559249_at | 593 | ATXN1 | 276.8801913 | 158.5949281 | 176.0 | 77.6 | 81.6 |
| 204286_s_at | 502 | PMAIP1 | 556.8716059 | 379.3762268 | 370.4 | 158.6 | 188.5 |
| 229204_at | 34 | HP1-BP74 | 91.88550229 | 21.78105698 | 297.9 | 176.7 | 116.6 |
| 219099_at | 642 | C12orf5 | 458.0108323 | 140.7303981 | 289.3 | 121.0 | 133.9 |
| 213183_s_at | 320 | CDKN1C | 53.81104837 | 23.77523442 | 48.2 | 31.9 | 20.0 |
| 218611_at | 560 | IER5 | 2094.876848 | 559.6799384 | 1453.6 | 609.3 | 709.8 |
| 228638_at | 348 | MGC34648 | 182.675226 | 64.22871143 | 172.4 | 88.8 | 68.9 |
| 1566403_at | 931 | RNU68 | 124.0391022 | 72.95277998 | 52.4 | 32.4 | 20.8 |
| 227897_at | 624 | RAP2B | 307.3313058 | 148.2233283 | 206.6 | 146.5 | 69.8 |
| 229397_s_at | 1017 | GRLF1 | 25.12088088 | 5.38543903 | 56.8 | 35.8 | 20.7 |
| 216609_at | 1008 | TXN | 18.2023911 | 4.083091107 | 79.7 | 64.9 | 25.2 |
| 222487_s_at | 17 | RPS27L | 63.3711197 | 18.47674156 | 254.3 | 142.8 | 95.4 |
| 228746_s_at | 704 | H41 | 84.49107472 | 54.63100835 | 58.6 | 52.2 | 16.5 |
| 230659_at | 1019 | EDEM1 | 36.79235659 | 22.52157399 | 37.3 | 26.1 | 11.4 |
| 213872_at | 886 | C6orf62 | 87.18546287 | 74.51211865 | 45.4 | 49.0 | 12.5 |
| 228030_at | 408 | RBM6 | 66.92784638 | 49.99393716 | 61.5 | 53.5 | 15.0 |
| 230333_at | 936 | SAT | 234.8209456 | 199.3058577 | 112.6 | 128.3 | 24.8 |
| 201694_s_at | 558 | EGR1 | 1065.285188 | 523.6056069 | 866.5 | 680.4 | 345.6 |
| 227404_s_at | 685 | EGR1 | 166.0581387 | 133.9676263 | 124.8 | 120.2 | 34.3 |
| 212834_at | 292 | DDX52 | 244.079041 | 86.75908123 | 270.5 | 108.5 | 87.8 |
| 231193_s_at | 3 | unknown | 17.49654557 | 3.314087173 | 96.4 | 66.9 | 24.1 |
| 1559343_at | 978 | UBE3A | 7.560112145 | 1.222277847 | 24.5 | 14.8 | 6.6 |
| 244546_at | 666 | CYCS | 301.7642436 | 118.4016892 | 192.9 | 113.9 | 58.2 |
| 220494_s_at | 618 | unknown | 112.3873397 | 101.6367982 | 81.8 | 60.9 | 19.3 |
| 232392_at | 910 | SFRS3 | 414.1451007 | 246.2652346 | 208.8 | 161.8 | 41.6 |
| 228834_at | 876 | TOB1 | 272.4410487 | 230.8314357 | 153.0 | 179.5 | 21.2 |
| 214395_x_at | 1006 | EEF1D | 96.3768725 | 25.79618187 | 348.5 | 288.0 | 68.6 |
| 214041_x_at | 1004 | RPL37A | 13.48090589 | 4.930513681 | 124.5 | 134.2 | 10.4 |

| | | | | | T-test |
|---|---|---|---|---|---|
| | | Gene/ | | Gene/ | P-value |
| | SD | GAPDH | Gene/ | GAPDH | Grp1 vs. |
| Probe set | Grp1 | Healthy | GAPDH | Grp1 | Other |
| Identifier | CIS | Controls | Other CIS | CIS | CIS |
| 217800_s_at | 209.6 | 16.90 | 79.07 | 237.42 | 9.79E−05 |
| 225247_at | 320.9 | 159.54 | 177.28 | 376.04 | 1.69E−03 |
| 213915_at | 1054.8 | 655.14 | 410.55 | 792.80 | 2.75E−02 |
| 200041_s_at | 840.1 | 303.71 | 668.87 | 1748.69 | 9.20E−13 |
| 211716_x_at | 157.4 | 87.92 | 88.71 | 214.99 | 7.47E−08 |
| 233350_s_at | 71.1 | 46.86 | 51.94 | 106.06 | 2.23E−04 |
| 219529_at | 42.5 | 19.28 | 10.99 | 21.77 | 7.21E−02 |
| 218607_s_at | 132.8 | 25.04 | 68.47 | 164.58 | 5.41E−09 |
| 202652_at | 76.4 | 32.03 | 39.12 | 80.12 | 8.52E−04 |
| 216520_s_at | 1252.3 | 537.40 | 1629.44 | 3344.55 | 4.83E−06 |
| 1554021_a_at | 23.6 | 5.36 | 15.58 | 35.15 | 8.96E−07 |
| 204122_at | 448.1 | 210.02 | 92.50 | 210.25 | 1.10E−02 |
| 219878_s_at | 53.6 | 41.02 | 32.19 | 61.98 | 1.12E−03 |
| 216231_s_at | 682.4 | 499.95 | 1308.66 | 2622.76 | 4.64E−06 |
| 219571_s_at | 20.3 | 4.17 | 12.60 | 26.21 | 1.33E−05 |
| 214450_at | 347.5 | 340.11 | 186.09 | 333.08 | 2.51E−02 |
| 209050_s_at | 163.0 | 220.49 | 180.83 | 335.52 | 2.54E−04 |
| 207088_s_at | 91.3 | 51.91 | 41.45 | 73.14 | 1.27E−02 |
| 1558215_s_at | 42.1 | 4.88 | 14.93 | 31.44 | 1.57E−04 |
| 200612_s_at | 48.4 | 24.23 | 18.91 | 35.30 | 4.71E−03 |
| 227266_s_at | 292.0 | 129.55 | 329.33 | 637.45 | 4.31E−07 |
| 205480_s_at | 305.9 | 99.16 | 271.81 | 492.34 | 2.61E−04 |
| 201831_s_at | 48.2 | 14.00 | 40.97 | 65.70 | 1.80E−02 |
| 232535_at | 21.8 | 3.04 | 9.62 | 16.64 | 1.09E−02 |
| 244042_x_at | 18.5 | 3.47 | 8.47 | 14.94 | 3.01E−03 |
| 233713_at | 33.3 | 6.21 | 21.83 | 40.39 | 2.51E−05 |
| 211947_s_at | 60.0 | 55.17 | 44.32 | 80.35 | 1.99E−05 |
| 212368_at | 176.3 | 36.44 | 110.47 | 196.97 | 1.05E−04 |
| 217854_s_at | 80.3 | 92.65 | 71.16 | 112.67 | 9.31E−03 |
| 217993_s_at | 516.9 | 138.48 | 492.32 | 808.13 | 1.03E−03 |
| 236562_at | 21.8 | 4.60 | 14.09 | 23.88 | 8.25E−04 |
| 200033_at | 443.0 | 562.18 | 1662.37 | 2784.71 | 3.15E−07 |
| 221895_at | 93.1 | 22.76 | 64.55 | 99.99 | 2.13E−02 |
| 201998_at | 216.5 | 65.32 | 193.61 | 306.62 | 3.94E−03 |
| 209458_x_at | 80.9 | 140.62 | 15.57 | 33.07 | 7.72E−03 |
| 241838_at | 28.0 | 3.21 | 12.37 | 20.99 | 6.91E−03 |
| 212540_at | 39.4 | 56.27 | 48.05 | 68.31 | 7.41E−02 |
| 212926_at | 49.3 | 14.69 | 46.12 | 70.75 | 3.53E−03 |
| 202283_at | 21.0 | 19.49 | 12.14 | 18.60 | 1.39E−02 |
| 200735_x_at | 866.5 | 723.57 | 2151.27 | 3405.33 | 1.11E−06 |
| 207460_at | 56.1 | 47.72 | 37.02 | 57.25 | 1.78E−02 |
| 211699_x_at | 72.2 | 100.97 | 18.94 | 36.30 | 4.12E−03 |
| 208635_x_at | 1066.2 | 693.07 | 2096.07 | 3286.00 | 1.64E−06 |
| 209651_at | 13.4 | 7.80 | 5.68 | 8.77 | 3.72E−02 |
| 213687_s_at | 919.9 | 764.68 | 2526.79 | 3815.75 | 7.09E−07 |
| 208325_s_at | 31.8 | 22.09 | 11.49 | 19.02 | 1.06E−02 |
| 203375_s_at | 103.7 | 36.84 | 101.01 | 149.53 | 8.79E−03 |
| 212063_at | 584.2 | 173.16 | 458.50 | 684.06 | 2.45E−03 |
| 235213_at | 782.6 | 136.19 | 506.64 | 750.43 | 1.00E−02 |
| 242778_at | 7.7 | 25.11 | 18.52 | 14.12 | 7.42E−04 |
| 226843_s_at | 138.1 | 333.89 | 248.54 | 182.23 | 2.62E−03 |
| 1569599_at | 2.1 | 4.98 | 5.36 | 3.65 | 1.12E−02 |
| 207111_at | 39.4 | 56.05 | 58.81 | 39.21 | 3.22E−02 |
| 242918_at | 2.1 | 21.69 | 4.39 | 2.97 | 2.19E−04 |
| 1553861_at | 15.1 | 13.85 | 16.54 | 11.96 | 1.54E−02 |
| 1552807_{'}a_at | 63.7 | 77.20 | 68.33 | 43.94 | 1.55E−02 |
| 238545_at | 78.9 | 195.21 | 150.80 | 92.15 | 1.34E−02 |
| 202381_at | 10.4 | 15.27 | 19.71 | 11.69 | 1.45E−02 |
| 226982_at | 99.3 | 78.41 | 111.38 | 66.26 | 7.50E−02 |
| 202083_s_at | 32.1 | 62.64 | 39.13 | 23.17 | 1.52E−02 |
| 228284_at | 28.6 | 39.12 | 31.64 | 19.43 | 6.86E−03 |
| 222670_s_at | 74.2 | 96.17 | 78.75 | 44.76 | 4.21E−02 |
| 243296_at | 83.2 | 59.92 | 77.47 | 50.45 | 8.67E−03 |
| 212840_at | 83.7 | 34.79 | 166.41 | 100.97 | 3.52E−05 |
| 1564164_at | 63.1 | 11.23 | 83.86 | 46.38 | 8.25E−03 |
| 204566_at | 51.1 | 62.27 | 97.71 | 53.43 | 7.81E−03 |
| 226643_s_at | 17.7 | 5.98 | 28.55 | 15.30 | 2.15E−03 |
| 228049_x_at | 36.1 | 77.44 | 101.62 | 55.66 | 1.99E−04 |
| 217602_at | 3.0 | 5.41 | 8.76 | 4.80 | 1.27E−02 |
| 215023_s_at | 5.0 | 11.28 | 15.05 | 7.11 | 4.52E−03 |
| 1567213_at | 98.8 | 37.65 | 176.71 | 98.66 | 8.98E−04 |
| 209160_at | 90.4 | 77.54 | 66.70 | 40.31 | 7.98E−02 |
| 205789_at | 5.1 | 8.59 | 12.61 | 5.41 | 2.27E−02 |
| 208750_s_at | 9.2 | 18.09 | 29.47 | 13.03 | 3.18E−02 |
| 201151_s_at | 24.4 | 8.63 | 49.61 | 23.02 | 1.20E−02 |
| 212649_at | 7.0 | 4.42 | 20.58 | 9.58 | 4.21E−04 |
| 229733_s_at | 3.4 | 13.45 | 14.77 | 6.64 | 1.18E−02 |
| 203603_s_at | 59.8 | 38.18 | 57.65 | 33.20 | 1.84E−02 |
| 201110_s_at | 3.8 | 10.84 | 9.06 | 3.71 | 1.19E−02 |
| 1555226_s_at | 9.1 | 15.70 | 22.76 | 8.90 | 1.90E−02 |
| 1559249_at | 30.2 | 48.39 | 54.78 | 27.17 | 3.23E−04 |
| 204286_s_at | 107.1 | 97.33 | 115.24 | 62.73 | 1.24E−03 |
| 229204_at | 23.1 | 16.06 | 92.70 | 38.82 | 1.34E−03 |
| 219099_at | 40.8 | 80.05 | 90.01 | 44.55 | 1.53E−04 |
| 213183_s_at | 4.6 | 9.41 | 15.01 | 6.65 | 4.84E−03 |
| 218611_at | 326.4 | 366.14 | 452.30 | 236.26 | 4.45E−04 |
| 228638_at | 19.2 | 31.93 | 53.64 | 22.93 | 3.90E−04 |
| 1566403_at | 6.9 | 21.68 | 16.31 | 6.92 | 2.23E−03 |
| 227897_at | 30.8 | 53.72 | 64.30 | 23.22 | 3.28E−03 |
| 229397_s_at | 5.7 | 4.39 | 17.68 | 6.90 | 1.60E−03 |
| 216609_at | 10.2 | 3.18 | 24.80 | 8.39 | 7.21E−03 |
| 222487_s_at | 45.2 | 11.08 | 79.12 | 31.76 | 7.44E−04 |
| 228746_s_at | 2.5 | 14.77 | 18.23 | 5.49 | 9.26E−03 |
| 230659_at | 2.3 | 6.43 | 11.60 | 3.79 | 1.81E−03 |
| 213872_at | 4.5 | 15.24 | 14.13 | 4.15 | 2.75E−02 |
| 228030_at | 2.6 | 11.70 | 19.13 | 4.99 | 5.39E−03 |
| 230333_at | 4.7 | 41.04 | 35.03 | 8.26 | 2.49E−02 |
| 201694_s_at | 314.0 | 186.19 | 269.63 | 115.03 | 1.78E−02 |
| 227404_s_at | 30.1 | 29.02 | 38.82 | 11.42 | 1.59E−02 |
| 212834_at | 31.0 | 42.66 | 84.17 | 29.23 | 2.80E−06 |
| 231193_s_at | 8.2 | 3.06 | 30.01 | 8.02 | 7.95E−04 |
| 1559343_at | 1.2 | 1.32 | 7.62 | 2.20 | 2.25E−04 |
| 244546_at | 36.9 | 52.74 | 60.02 | 19.39 | 3.91E−04 |
| 220494_s_at | 5.1 | 19.64 | 25.46 | 6.43 | 1.31E−03 |
| 232392_at | 15.3 | 72.38 | 64.97 | 13.86 | 1.23E−03 |
| 228834_at | 8.1 | 47.62 | 47.62 | 7.05 | 1.67E−02 |
| 214395_x_at | 22.4 | 16.84 | 108.44 | 22.82 | 2.15E−03 |
| 214041_x_at | 4.1 | 2.36 | 38.75 | 3.48 | 6.32E−03 |

TABLE 1B

| | | | | | | | | | Gene/ | Gene/ | Gene/ | T-test P- |
|---|---|---|---|---|---|---|---|---|---|---|---|---|
| | | | | | Ave. | SD | Ave. | SD | GAPDH | GAPDH | GAPDH | value |
| Probe set | SEQ ID | Marker | Ave. Healthy | SD | Other | Other | Grp1 | Grp1 | Healthy | Other | Grp1 | Grp1 vs. |
| Identifier | NO: | Gene | Control | Control | CIS | CIS | CIS | CIS | Controls | CIS | CIS | Other CIS |
| AFFX- | 1022 | GAPDH | 5721.51139 | 1112.780879 | 3213.7 | 1181.0 | 3004.4 | 822.7 | 1000 | 1000 | 1000 | 5.90E−01 |
| HUMGAPDH/ | | | | | | | | | | | | |
| M33197_3_at | | | | | | | | | | | | |

TABLE 2

| | SEQ | | | | Ave. | SD | Ave. |
|---|---|---|---|---|---|---|---|
| Probe set | ID | Marker | Ave. Healthy | SD | Other | Other | Grp1 |
| Identifier | NO: | Gene | Control | Control | CIS | CIS | CIS |
| 1555981_at | 977 | C17orf65 | 38.95432822 | 15.34203 | 36.2 | 19.1 | 88.4 |
| 201151_s_at | 983 | MBNL1 | 49.3888033 | 17.65848 | 159.4 | 115.2 | 69.2 |
| 201369_s_at | 985 | ZFP36L2 | 324.3180809 | 179.1896 | 386.8 | 316.2 | 1259.6 |
| 204355_at | 989 | DHX30 | 165.7049361 | 74.02059 | 87.0 | 78.1 | 155.2 |
| 204443_at | 311 | ARSA | 149.3298935 | 53.38473 | 83.5 | 52.9 | 181.8 |
| 207238_s_at | 287 | PTPRC | 240.7214311 | 229.6297 | 325.1 | 120.8 | 449.7 |
| 207460_at | 617 | GZMM | 273.0278916 | 93.21375 | 119.0 | 60.6 | 172.0 |
| 207688_s_at | 993 | INHBC | 47.70860316 | 13.49079 | 184.9 | 131.1 | 42.4 |
| 208700_s_at | 994 | TKT | 773.596628 | 190.5899 | 318.6 | 131.4 | 426.8 |
| 208751_at | 995 | NAPA | 57.24012334 | 22.75417 | 36.0 | 18.9 | 97.3 |
| 208764_s_at | 996 | ATP5G2 | 1064.023434 | 298.7852 | 1031.5 | 549.3 | 2754.1 |
| 209398_at | 941 | HIST1H1C | 202.0854177 | 77.05926 | 65.4 | 31.1 | 69.6 |
| 212044_s_at | 1002 | RPL27A | 315.1552812 | 74.41744 | 796.7 | 569.0 | 236.5 |
| 213183_s_at | 320 | CDKN1C | 53.81104837 | 23.77523 | 48.2 | 31.9 | 20.0 |
| 214123_s_at | 1005 | C4orf10 | 50.3753172 | 19.53119 | 129.7 | 117.6 | 31.0 |
| 214395_x_at | 1006 | EEF1D | 96.3768725 | 25.79618 | 348.5 | 288.0 | 68.6 |
| 216520_s_at | 138 | TPT1 | 3074.723831 | 3156.269 | 5236.5 | 2877.9 | 10048.5 |
| 217257_at | 1009 | unknown | 23.65021404 | 4.594125 | 97.2 | 66.4 | 23.3 |
| 221943_x_at | 1012 | RPL38 | 408.5680429 | 139.3603 | 1035.3 | 747.2 | 273.9 |
| 221960_s_at | 1013 | RAB2 | 71.96527979 | 21.04722 | 130.3 | 77.2 | 48.8 |
| 222487_s_at | 17 | RPS27L | 63.3711197 | 18.47674 | 254.3 | 142.8 | 95.4 |
| 225247_at | 298 | C19orf6 | 912.8038675 | 351.313 | 569.7 | 511.6 | 1129.8 |
| 227259_at | 1015 | CD47 | 350.7451938 | 200.3456 | 341.6 | 202.8 | 236.2 |
| 228673_s_at | 1016 | EML4 | 117.0116781 | 48.07124 | 225.5 | 167.8 | 62.1 |
| 229543_at | 1018 | RPL29 | 137.5338645 | 66.27801 | 394.7 | 419.9 | 111.9 |
| 238761_at | 245 | unknown | 75.96646062 | 49.14559 | 169.6 | 98.7 | 98.1 |
| 239487_at | 1020 | FAM98A | 59.11653508 | 32.00945 | 101.1 | 35.9 | 57.2 |
| 241617_x_at | 1021 | unknown | 72.37997255 | 13.27325 | 149.0 | 127.2 | 43.2 |

| | | Gene/ | Gene/ | Gene/ | T-test |
|---|---|---|---|---|---|
| | SD | GAPDH | GAPDH | GAPDH | P-value |
| Probe set | Grp1 | Healthy | Other | Grp1 | Grp1 vs. |
| Identifier | CIS | Controls | CIS | CIS | Other CIS |
| 1555981_at | 22.5 | 6.81 | 11.27 | 29.43 | 4.10E−08 |
| 201151_s_at | 24.4 | 8.63 | 49.61 | 23.02 | 1.20E−02 |
| 201369_s_at | 459.4 | 56.68 | 120.35 | 419.26 | 2.27E−07 |
| 204355_at | 38.8 | 28.96 | 27.08 | 51.67 | 8.09E−03 |
| 204443_at | 53.6 | 26.10 | 26.00 | 60.52 | 1.28E−05 |
| 207238_s_at | 141.0 | 42.07 | 101.17 | 149.68 | 1.07E−02 |
| 207460_at | 56.1 | 47.72 | 37.02 | 57.25 | 1.78E−02 |
| 207688_s_at | 16.0 | 8.34 | 57.53 | 14.11 | 7.56E−04 |
| 208700_s_at | 123.4 | 135.21 | 99.15 | 142.06 | 2.55E−02 |
| 208751_at | 29.2 | 10.00 | 11.19 | 32.37 | 1.82E−08 |
| 208764_s_at | 430.4 | 185.97 | 320.98 | 916.66 | 1.05E−10 |
| 209398_at | 44.5 | 35.32 | 20.35 | 23.17 | 7.48E−01 |
| 212044_s_at | 58.7 | 55.08 | 247.92 | 78.73 | 1.94E−03 |
| 213183_s_at | 4.6 | 9.41 | 15.01 | 6.65 | 4.84E−03 |
| 214123_s_at | 7.4 | 8.80 | 40.36 | 10.31 | 6.97E−03 |
| 214395_x_at | 22.4 | 16.84 | 108.44 | 22.82 | 2.15E−03 |
| 216520_s_at | 1252.3 | 537.40 | 1629.44 | 3344.55 | 4.83E−06 |
| 217257_at | 6.9 | 4.13 | 30.25 | 7.77 | 5.84E−04 |
| 221943_x_at | 89.3 | 71.41 | 322.14 | 91.17 | 1.42E−03 |
| 221960_s_at | 13.7 | 12.58 | 40.53 | 16.24 | 1.05E−03 |
| 222487_s_at | 45.2 | 11.08 | 79.12 | 31.76 | 7.44E−04 |
| 225247_at | 320.9 | 159.54 | 177.28 | 376.04 | 1.69E−03 |
| 227259_at | 163.9 | 61.30 | 106.28 | 78.61 | 1.33E−01 |
| 228673_s_at | 18.7 | 20.45 | 70.18 | 20.66 | 2.14E−03 |
| 229543_at | 81.7 | 24.04 | 122.81 | 37.26 | 2.86E−02 |
| 238761_at | 20.3 | 13.28 | 52.78 | 32.64 | 1.92E−02 |
| 239487_at | 33.4 | 10.33 | 31.47 | 19.05 | 1.43E−03 |
| 241617_x_at | 12.3 | 12.65 | 46.37 | 14.39 | 7.50E−03 |

TABLE 4

| | SEQ | | | | | | |
|---|---|---|---|---|---|---|---|
| Probe set | ID | | | Ave. | Ave. | | |
| Identifier | NO: | Marker Gene | Gene Description | Ctrl | CIS | Ratio | P-value |
| 237383_at | 1 | | Transcribed locus | 30.5 | 143.8 | 4.72 | <1.0E−07 |
| 217800_s_at | 2 | NDFIP1 | Nedd4 family interacting protein 1 | 102.3 | 454.6 | 4.44 | <1.0E−07 |
| 231193_s_at | 3 | | CDNA clone IMAGE: 4285619 | 18.5 | 69.4 | 3.74 | 1.0E−07 |
| 226458_at | 4 | | clone IMAGE: 4449283 | 61.0 | 221.0 | 3.62 | 1.0E−07 |
| 202342_s_at | 5 | TRIM2 | tripartite motif-containing 2 | 17.6 | 62.5 | 3.56 | <1.0E−07 |
| 226035_at | 6 | USP31 | ubiquitin specific peptidase 31 | 29.0 | 98.9 | 3.41 | <1.0E−07 |
| 233085_s_at | 7 | FLJ22833 | hypothetical protein FLJ22833 | 113.0 | 380.5 | 3.37 | <1.0E−07 |
| 230085_at | 8 | | Transcribed locus | 115.1 | 383.1 | 3.33 | 7.4E−06 |
| 238355_at | 9 | RNPC2 | RNA-binding region (RNP1, RRM) containing 2 | 10.6 | 34.5 | 3.26 | 4.0E−07 |
| 209662_at | 10 | CETN3 | centrin, EF-hand protein, 3 (CDC31 homolog, yeast) | 104.9 | 341.8 | 3.26 | <1.0E−07 |
| 1564164_at | 11 | FLJ20054 | hypothetical protein FLJ20054 | 68.1 | 221.5 | 3.25 | <1.0E−07 |
| 202600_s_at | 12 | NRIP1 | nuclear receptor interacting protein 1 | 64.4 | 209.3 | 3.25 | <1.0E−07 |
| 224851_at | 13 | CDK6 | cyclin-dependent kinase 6 | 134.8 | 427.8 | 3.17 | <1.0E−07 |
| 230860_at | 14 | | Transcribed locus | 36.5 | 115.6 | 3.17 | <1.0E−07 |
| 1556064_at | 15 | LOC284926 | Hypothetical protein LOC284926 | 11.6 | 36.8 | 3.16 | <1.0E−07 |
| 1557238_s_at | 16 | FLJ10707 | Hypothetical protein FLJ10707 | 11.8 | 37.3 | 3.15 | <1.0E−07 |
| 222487_s_at | 17 | RPS27L | ribosomal protein S27-like | 64.8 | 203.5 | 3.14 | <1.0E−07 |
| 1559500_at | 18 | KIAA0804 | KIAA0804 | 21.7 | 67.2 | 3.10 | <1.0E−07 |
| 213530_at | 19 | RAB3GAP1 | RAB3 GTPase activating protein subunit 1 (catalytic) | 59.6 | 182.7 | 3.07 | <1.0E−07 |
| 225537_at | 20 | TRAPPC6B | trafficking protein particle complex 6B | 66.7 | 202.7 | 3.04 | <1.0E−07 |
| 222872_x_at | 21 | FLJ22833 | hypothetical protein FLJ22833 | 205.9 | 621.0 | 3.02 | <1.0E−07 |
| 203129_s_at | 22 | KIF5C | kinesin family member 5C | 136.3 | 407.0 | 2.99 | <1.0E−07 |
| 215898_at | 23 | TTLL5 | tubulin tyrosine ligase-like family, member 5 | 26.8 | 80.1 | 2.99 | <1.0E−07 |
| 239205_s_at | 24 | CR1 /// CR1L | complement component (3b/4b) receptor 1 | 24.0 | 71.0 | 2.96 | 5.0E−07 |
| 240168_at | 25 | XPO7 | exportin 7 | 119.5 | 350.1 | 2.93 | <1.0E−07 |
| 230323_s_at | 26 | TMEM45B | transmembrane protein 45B | 48.6 | 137.9 | 2.84 | <1.0E−07 |
| 1558250_s_at | 27 | | DKFZp686E16168 | 113.8 | 320.2 | 2.81 | <1.0E−07 |
| 242557_at | 28 | | Transcribed locus | 97.5 | 272.0 | 2.79 | 1.0E−07 |
| 223698_at | 29 | SLC25A36 | solute carrier family 25, member 36 | 25.4 | 70.7 | 2.78 | <1.0E−07 |
| 226977_at | 30 | LOC492311 | similar to bovine IgA regulatory protein | 268.8 | 746.6 | 2.78 | <1.0E−07 |
| 1552343_s_at | 31 | PDE7A | phosphodiesterase 7A | 95.0 | 258.6 | 2.72 | <1.0E−07 |
| 218820_at | 32 | C14orf132 | chromosome 14 open reading frame 132 | 107.1 | 290.7 | 2.71 | <1.0E−07 |
| 244517_x_at | 33 | RNF146 | Ring finger protein 146 | 63.4 | 170.2 | 2.68 | <1.0E−07 |
| 229204_at | 34 | HP1-BP74 | Heterochromatin protein 1, binding protein 3 | 93.2 | 249.2 | 2.67 | 7.0E−07 |
| 236165_at | 35 | MSL3L1 | male-specific lethal 3-like 1 (Drosophila) | 102.6 | 274.2 | 2.67 | <1.0E−07 |
| 236450_at | 36 | TARSL2 | Threonyl-tRNA synthetase-like 2 | 18.9 | 50.6 | 2.67 | <1.0E−07 |
| 210077_s_at | 37 | SFRS5 | splicing factor, arginine/serine-rich 5 | 42.3 | 112.3 | 2.66 | <1.0E−07 |
| 243981_at | 38 | STK4 | serine/threonine kinase 4 | 174.9 | 461.5 | 2.64 | <1.0E−07 |
| 225414_at | 39 | RNF149 | ring finger protein 149 | 243.1 | 638.9 | 2.63 | <1.0E−07 |
| 241838_at | 40 | | Transcribed locus | 20.6 | 54.0 | 2.63 | <1.0E−07 |
| 224558_s_at | 41 | MALAT1 | metastasis associated lung adenocarcinoma transcript 1 | 1851.8 | 4857.9 | 2.62 | <1.0E−07 |
| 217536_x_at | 42 | | weakly similar to NP_055301.1 neuronal thread protein | 44.2 | 116.0 | 2.62 | <1.0E−07 |
| | | | AD7c-NTP | | | | |
| 1558233_s_at | 43 | ATF1 | Activating transcription factor 1 | 63.7 | 166.8 | 2.62 | <1.0E−07 |
| 239449_at | 44 | ANKH | Ankylosis, progressive homolog (mouse) | 53.1 | 139.0 | 2.62 | <1.0E−07 |
| 239441_at | 45 | | | 21.1 | 55.1 | 2.61 | 1.1E−05 |
| 204373_s_at | 46 | CAP350 | centrosome-associated protein 350 | 465.7 | 1210.3 | 2.60 | <1.0E−07 |
| 223243_s_at | 47 | C1orf22 | chromosome 1 open reading frame 22 | 156.1 | 402.5 | 2.58 | <1.0E−07 |
| 237387_at | 48 | | Transcribed locus | 21.9 | 56.4 | 2.57 | <1.0E−07 |
| 227871_at | 49 | CHM | choroideremia (Rab escort protein 1) | 318.8 | 819.9 | 2.57 | <1.0E−07 |
| 229007_at | 50 | LOC283788 | hypothetical protein LOC283788 | 29.5 | 75.6 | 2.56 | 7.5E−06 |
| 226041_at | 51 | NAPE-PLD | N-acyl-phosphatidylethanolamine-hydrolyzing | 65.5 | 167.1 | 2.55 | 1.0E−07 |
| | | | phospholipase D | | | | |
| 228734_at | 52 | UBE2V2 | Ubiquitin-conjugating enzyme E2 variant 2 | 19.3 | 49.2 | 2.54 | 1.5E−05 |
| 217655_at | 53 | FXYD5 | FXYD domain containing ion transport regulator 5 | 63.6 | 161.1 | 2.53 | 6.0E−07 |
| 212215_at | 54 | PREPL | prolyl endopeptidase-like | 190.8 | 483.2 | 2.53 | <1.0E−07 |
| 244521_at | 55 | C20orf17 | Chromosome 20 open reading frame 17 | 38.8 | 98.2 | 2.53 | <1.0E−07 |
| 218643_s_at | 56 | CRIPT | postsynaptic protein CRIPT | 56.5 | 142.9 | 2.53 | <1.0E−07 |
| 229366_at | 57 | CRBN | Cereblon | 115.0 | 290.5 | 2.53 | <1.0E−07 |
| 220459_at | 58 | MCM3APAS | MCM3 minichromosome maintenance deficient 3 (S. cerevisiae) | 22.9 | 57.7 | 2.52 | <1.0E−07 |
| | | | associated protein antisense | | | | |
| 219308_s_at | 59 | AK5 | adenylate kinase 5 | 90.9 | 228.8 | 2.52 | <1.0E−07 |
| 219467_at | 60 | FLJ20125 | hypothetical protein FLJ20125 | 102.7 | 258.6 | 2.52 | <1.0E−07 |
| 222714_s_at | 61 | LACTB2 | lactamase, beta 2 | 14.6 | 36.8 | 2.52 | <1.0E−07 |
| 230556_at | 62 | IMMP1L | IMP1 inner mitochondrial membrane peptidase-like (S. cerevisiae) | 37.6 | 94.2 | 2.51 | <1.0E−07 |
| 1554480_a_at | 63 | SVH | SVH protein | 105.5 | 263.4 | 2.50 | <1.0E−07 |
| 237464_at | 64 | IMAA | LAT1-3TM protein 2 | 77.1 | 192.2 | 2.49 | <1.0E−07 |
| 1561195_at | 65 | TM7SF1 | Transmembrane 7 superfamily member 1 (upregulated in | 10.9 | 27.0 | 2.48 | <1.0E−07 |
| | | | kidney) | | | | |
| 239888_at | 66 | | Transcribed locus | 12.7 | 31.4 | 2.48 | 1.0E−07 |
| 229070_at | 67 | C6orf105 | chromosome 6 open reading frame 105 | 229.8 | 570.7 | 2.48 | <1.0E−07 |
| 239577_at | 68 | | | 46.2 | 114.7 | 2.48 | <1.0E−07 |
| 213149_at | 69 | DLAT | dihydrolipoamide S-acetyltransferase (E2 component of | 25.8 | 64.1 | 2.48 | <1.0E−07 |
| | | | pyruvate dehydrogenase complex) | | | | |
| 224786_at | 70 | SCOC | short coiled-coil protein | 122.2 | 302.9 | 2.48 | <1.0E−07 |
| 1558215_s_at | 71 | UBTF | upstream binding transcription factor, RNA polymerase I | 28.3 | 70.3 | 2.48 | <1.0E−07 |
| 213268_at | 72 | CAMTA1 | calmodulin binding transcription activator 1 | 21.2 | 52.6 | 2.48 | 3.0E−07 |
| 218967_s_at | 73 | PTER | phosphotriesterase related | 54.3 | 134.4 | 2.47 | <1.0E−07 |
| 235427_at | 74 | | Transcribed locus | 150.8 | 372.3 | 2.47 | <1.0E−07 |
| 231956_at | 75 | KIAA1618 | KIAA1618 | 91.0 | 224.5 | 2.47 | <1.0E−07 |
| 235213_at | 76 | ITPKB | Inositol 1,4,5-trisphosphate 3-kinase B | 799.3 | 1965.6 | 2.46 | <1.0E−07 |
| 229287_at | 77 | | Full-length cDNA clone CS0DK010YA20 of HeLa cells Cot | 212.7 | 522.6 | 2.46 | <1.0E−07 |
| | | | 25-normalized of Homo sapiens | | | | |
| 200056_s_at | 78 | C1D | nuclear DNA-binding protein /// nuclear DNA-binding | 169.1 | 415.0 | 2.46 | <1.0E−07 |
| | | | protein | | | | |
| 206061_s_at | 79 | DICER1 | Dicer1, Dcr-1 homolog (Drosophila) | 114.9 | 281.7 | 2.45 | 3.0E−07 |
| 228446_at | 80 | KIAA2026 | KIAA2026 | 460.5 | 1118.7 | 2.43 | <1.0E−07 |
| 1557055_s_at | 81 | LOC284591 | hypothetical protein LOC284591 | 159.0 | 385.7 | 2.43 | 1.0E−07 |
| 232628_at | 82 | FAM13A1 | Family with sequence similarity 13, member A1 | 110.2 | 267.2 | 2.43 | 3.8E−06 |
| 1554021_a_at | 83 | ZNF12 | zinc finger protein 12 | 29.6 | 71.3 | 2.41 | <1.0E−07 |
| 240824_at | 84 | OBFC1 | Chromosome 21 open reading frame 53 | 68.9 | 165.6 | 2.40 | <1.0E−07 |
| 231866_at | 85 | LNPEP | leucyl/cystinyl aminopeptidase | 386.2 | 927.8 | 2.40 | <1.0E−07 |
| 215985_at | 86 | HCG8 | HLA complex group 8 | 64.7 | 155.3 | 2.40 | <1.0E−07 |
| 213317_at | 87 | CLIC5 | Chloride intracellular channel 5 | 42.1 | 100.9 | 2.39 | <1.0E−07 |
| 233713_at | 88 | SMYD2 | SET and MYND domain containing 2 | 36.5 | 87.3 | 2.39 | <1.0E−07 |
| 209840_s_at | 89 | LRRN3 | leucine rich repeat neuronal 3 | 368.5 | 879.7 | 2.39 | 2.9E−06 |
| 240513_at | 90 | | | 36.6 | 87.0 | 2.38 | <1.0E−07 |
| 1558732_at | 91 | | | 186.7 | 442.5 | 2.37 | <1.0E−07 |
| 206641_at | 92 | TNFRSF17 | tumor necrosis factor receptor superfamily, member 17 | 23.1 | 54.6 | 2.36 | 1.9E−03 |
| 1556543_at | 93 | ZCCHC7 | Zinc finger, CCHC domain containing 7 | 72.3 | 170.9 | 2.36 | <1.0E−07 |
| 223324_s_at | 94 | TRPM7 | transient receptor potential cation channel, subfamily M, | 63.1 | 148.3 | 2.35 | <1.0E−07 |
| | | | member 7 | | | | |
| 236000_s_at | 95 | HNRPD | Heterogeneous nuclear ribonucleoprotein D (AU-rich | 322.8 | 758.1 | 2.35 | <1.0E−07 |
| | | | element RNA binding protein 1, 37 kDa) | | | | |
| 217993_s_at | 96 | MAT2B | methionine adenosyltransferase II, beta | 820.4 | 1919.3 | 2.34 | <1.0E−07 |
| 225127_at | 97 | KIAA1423 | KIAA1423 | 179.8 | 420.4 | 2.34 | <1.0E−07 |
| 241741_at | 98 | C20orf155 | chromosome 20 open reading frame 155 | 52.1 | 121.7 | 2.34 | <1.0E−07 |
| 213353_at | 99 | ABCA5 | ATP-binding cassette, sub-family A (ABC1), member 5 | 176.2 | 411.5 | 2.33 | <1.0E−07 |
| 239726_at | 100 | | | 156.3 | 364.7 | 2.33 | <1.0E−07 |
| 225835_at | 101 | SLC12A2 | solute carrier family 12 (sodium/potassium/chloride | 49.5 | 115.2 | 2.33 | <1.0E−07 |
| | | | transporters), member 2 | | | | |
| 232535_at | 102 | | MRNA; cDNA DKFZp434L201 (from clone | 19.0 | 44.1 | 2.32 | 9.0E−07 |
| | | | DKFZp434L201) | | | | |
| 233898_s_at | 103 | FGFR1OP2 | FGFR1 oncogene partner 2 | 693.7 | 1606.0 | 2.32 | <1.0E−07 |
| 212840_at | 104 | KIAA0794 | KIAA0794 protein | 190.9 | 440.8 | 2.31 | <1.0E−07 |
| 238635_at | 105 | FLJ21657 | hypothetical protein FLJ21657 | 11.3 | 26.1 | 2.31 | 2.0E−07 |
| 219571_s_at | 106 | ZNF12 | zinc finger protein 12 | 25.0 | 57.4 | 2.30 | 1.0E−07 |
| 1554345_a_at | 107 | FLJ20125 | hypothetical protein FLJ20125 | 35.0 | 80.1 | 2.29 | 3.0E−07 |
| 238598_s_at | 108 | RNF32 | Ring finger protein 32 | 56.7 | 129.8 | 2.29 | <1.0E−07 |
| 205763_s_at | 109 | DDX18 | DEAD (Asp-Glu-Ala-Asp) box polypeptide 18 | 116.5 | 266.5 | 2.29 | <1.0E−07 |
| 242827_x_at | 110 | | Transcribed locus | 134.4 | 307.3 | 2.29 | <1.0E−07 |
| 225240_s_at | 111 | MSI2 | musashi homolog 2 (Drosophila) | 363.7 | 830.8 | 2.28 | <1.0E−07 |
| 218135_at | 112 | PTX1 | PTX1 protein | 77.3 | 176.3 | 2.28 | <1.0E−07 |
| 238174_at | 113 | FBXL17 | F-box and leucine-rich repeat protein 17 | 23.6 | 53.8 | 2.28 | 2.5E−05 |
| 232165_at | 114 | EPPK1 | epiplakin 1 | 142.9 | 325.6 | 2.28 | 1.0E−07 |
| 221830_at | 115 | RAP2A | RAP2A, member of RAS oncogene family | 225.4 | 510.5 | 2.27 | <1.0E−07 |
| 218607_s_at | 116 | SDAD1 | SDA1 domain containing 1 | 153.8 | 346.7 | 2.25 | <1.0E−07 |
| 228157_at | 117 | ZNF207 | zinc finger protein 207 | 469.4 | 1055.0 | 2.25 | <1.0E−07 |
| 232974_at | 118 | HDHD1A | Haloacid dehalogenase-like hydrolase domain containing 1A | 101.2 | 227.3 | 2.25 | <1.0E−07 |
| 202351_at | 119 | ITGAV | integrin, alpha V (vitronectin receptor, alpha polypeptide, | 196.1 | 440.2 | 2.24 | <1.0E−07 |
| | | | antigen CD51) | | | | |
| 228248_at | 120 | AVO3 | TORC2-specific protein AVO3 | 97.5 | 218.4 | 2.24 | 2.9E−05 |
| 226329_s_at | 121 | LOC129531 | hypothetical protein BC018453 | 126.8 | 284.0 | 2.24 | <1.0E−07 |
| 205097_at | 122 | SLC26A2 | solute carrier family 26 (sulfate transporter), member 2 | 63.1 | 140.9 | 2.23 | <1.0E−07 |
| 230192_at | 123 | RFP2 | ret finger protein 2 | 46.7 | 104.3 | 2.23 | 2.4E−06 |
| 236835_at | 124 | FUT8 | fucosyltransferase 8 (alpha (1,6) fucosyltransferase) | 21.8 | 48.6 | 2.23 | <1.0E−07 |
| 213331_s_at | 125 | NEK1 | NIMA (never in mitosis gene a)-related kinase 1 | 101.4 | 226.3 | 2.23 | <1.0E−07 |
| 237006_at | 126 | MLLT2 | AF4/FMR2 family, member 1 | 196.8 | 439.1 | 2.23 | <1.0E−07 |
| 202760_s_at | 127 | PALM2-AKAP2 | PALM2-AKAP2 protein | 124.1 | 276.5 | 2.23 | <1.0E−07 |
| 201737_s_at | 128 | MARCH6 | membrane-associated ring finger (C3HC4) 6 | 168.8 | 375.7 | 2.23 | <1.0E−07 |
| 235302_at | 129 | | Full-length cDNA clone CS0CAP006YP08 of Thymus of | 65.8 | 146.3 | 2.22 | <1.0E−07 |
| | | | Homo sapiens (human) | | | | |
| 1557733_a_at | 130 | | MRNA; cDNA DKFZp667K2218 (from clone | 648.3 | 1441.2 | 2.22 | <1.0E−07 |
| | | | DKFZp667K2218) | | | | |
| 217196_s_at | 131 | CAMSAP1L1 | calmodulin regulated spectrin-associated protein 1-like 1 | 65.1 | 144.7 | 2.22 | <1.0E−07 |
| 211761_s_at | 132 | CACYBP | calcyclin binding protein /// calcyclin binding protein | 478.0 | 1061.0 | 2.22 | <1.0E−07 |
| 244414_at | 133 | MAML2 | Mastermind-like 2 (Drosophila) | 419.7 | 931.4 | 2.22 | <1.0E−07 |
| 205292_s_at | 134 | HNRPA2B1 | heterogeneous nuclear ribonucleoprotein A2/B1 | 772.3 | 1711.6 | 2.22 | <1.0E−07 |
| 1565830_at | 135 | KIAA1731 | KIAA1731 | 19.6 | 43.5 | 2.21 | 4.8E−06 |
| 206314_at | 136 | ZNF167 | zinc finger protein 167 | 93.5 | 207.1 | 2.21 | <1.0E−07 |
| 238577_s_at | 137 | | Transcribed locus | 74.5 | 164.6 | 2.21 | <1.0E−07 |
| 216520_s_at | 138 | TPT1 | tumor protein, translationally-controlled 1 | 3119.4 | 6895.4 | 2.21 | 1.0E−07 |
| 223681_s_at | 139 | INADL | InaD-like (Drosophila) | 25.0 | 55.3 | 2.21 | <1.0E−07 |
| 235306_at | 140 | GIMAP8 | GTPase, IMAP family member 8 | 249.0 | 548.3 | 2.20 | 1.0E−07 |
| 228190_at | 141 | | | 41.9 | 92.3 | 2.20 | 1.1E−05 |
| 241891_at | 142 | DOCK8 | Dedicator of cytokinesis 8 | 84.8 | 185.8 | 2.19 | <1.0E−07 |
| 219004_s_at | 143 | C21orf45 | chromosome 21 open reading frame 45 | 301.5 | 660.2 | 2.19 | <1.0E−07 |
| 226587_at | 144 | SNRPN | Small nuclear ribonucleoprotein polypeptide N | 71.9 | 157.3 | 2.19 | <1.0E−07 |
| 218361_at | 145 | GOLPH3L | golgi phosphoprotein 3-like | 139.2 | 304.1 | 2.19 | 8.0E−07 |
| 240182_at | 146 | | Transcribed locus, strongly similar to XP_531023.1 | 50.7 | 110.9 | 2.19 | 5.0E−04 |
| 235728_at | 147 | ZFP3 | zinc finger protein 3 homolog (mouse) | 70.2 | 153.1 | 2.18 | 2.0E−07 |
| 222791_at | 148 | RSBN1 | round spermatid basic protein 1 | 112.3 | 244.8 | 2.18 | <1.0E−07 |
| 219979_s_at | 149 | HSPC138 | hypothetical protein HSPC138 | 46.0 | 100.3 | 2.18 | 4.4E−05 |
| 226503_at | 150 | RIF1 | RAP1 interacting factor homolog (yeast) | 98.1 | 213.0 | 2.17 | <1.0E−07 |
| 231896_s_at | 151 | DENR | density-regulated protein | 664.7 | 1442.1 | 2.17 | <1.0E−07 |
| 212368_at | 152 | ZNF292 | zinc finger protein 292 | 208.4 | 451.9 | 2.17 | <1.0E−07 |
| 1569607_s_at | 153 | ANKRD20A2 | ankyrin repeat domain 20 family, member A2 | 172.8 | 374.6 | 2.17 | 2.2E−06 |
| 236562_at | 154 | ZNF439 | zinc finger protein 439 | 26.2 | 56.7 | 2.17 | <1.0E−07 |
| 229075_at | 155 | | Transcribed locus | 54.7 | 118.5 | 2.16 | 6.7E−06 |
| 225100_at | 156 | FBXO45 | F-box protein 45 | 145.9 | 314.0 | 2.15 | <1.0E−07 |
| 243363_at | 157 | LEF1 | lymphoid enhancer-binding factor 1 | 135.1 | 290.3 | 2.15 | <1.0E−07 |
| 203870_at | 158 | USP46 | ubiquitin specific peptidase 46 | 111.4 | 239.0 | 2.15 | 2.7E−05 |
| 229333_at | 159 | | Transcribed locus, weakly similar to XP_512541.1 | 79.0 | 169.3 | 2.14 | 2.5E−06 |
| 229531_at | 160 | | Mitochondrial carrier triple repeat 6 | 62.1 | 133.1 | 2.14 | <1.0E−07 |
| 220235_s_at | 161 | C1orf103 | chromosome 1 open reading frame 103 | 76.0 | 162.8 | 2.14 | 1.0E−07 |
| 232333_at | 162 | MAML2 | Mastermind-like 2 (Drosophila) | 116.9 | 249.9 | 2.14 | <1.0E−07 |
| 228729_at | 163 | CCNB1 | cyclin B1 | 15.6 | 33.3 | 2.14 | 1.9E−05 |
| 212926_at | 164 | SMC5L1 | SMC5 structural maintenance of chromosomes 5-like 1 | 87.2 | 186.3 | 2.14 | <1.0E−07 |
| | | | (yeast) | | | | |
| 213687_s_at | 165 | RPL35A | ribosomal protein L35a | 4596.7 | 9812.4 | 2.13 | <1.0E−07 |
| 201143_s_at | 166 | EIF2S1 | eukaryotic translation initiation factor 2, subunit 1 alpha, | 239.8 | 511.5 | 2.13 | 1.0E−07 |
| | | | 35 kDa | | | | |
| 238653_at | 167 | LRIG2 | Leucine-rich repeats and immunoglobulin-like domains 2 | 153.0 | 326.0 | 2.13 | <1.0E−07 |
| 223264_at | 168 | MESDC1 | mesoderm development candidate 1 | 395.9 | 843.2 | 2.13 | <1.0E−07 |
| 230494_at | 169 | | | 206.3 | 438.5 | 2.13 | <1.0E−07 |
| 227751_at | 170 | PDCD5 | Programmed cell death 5 | 36.7 | 77.9 | 2.12 | <1.0E−07 |
| 1569827_at | 171 | APG7L | ATG7 autophagy related 7 homolog (S. cerevisiae) | 38.1 | 80.8 | 2.12 | <1.0E−07 |
| 1560926_at | 172 | PPP4R2 | Protein phosphatase 4, regulatory subunit 2 | 63.8 | 135.0 | 2.12 | 2.3E−04 |
| 244663_at | 173 | | Transcribed locus | 48.5 | 102.7 | 2.12 | 3.0E−07 |
| 205449_at | 174 | SAC3D1 | SAC3 domain containing 1 | 109.7 | 232.0 | 2.12 | 5.5E−05 |
| 211276_at | 175 | TCEAL2 | transcription elongation factor A (SII)-like 2 | 48.2 | 101.9 | 2.11 | 8.0E−06 |
| 238944_at | 176 | | Transcribed locus, moderately similar to XP_512724.1 | 181.3 | 381.7 | 2.11 | 2.0E−07 |
| 238468_at | 177 | TNRC6B | trinucleotide repeat containing 6B | 289.3 | 608.0 | 2.10 | 1.0E−07 |
| 203983_at | 178 | TSNAX | translin-associated factor X | 279.6 | 584.8 | 2.09 | <1.0E−07 |
| 218943_s_at | 179 | DDX58 | DEAD (Asp-Glu-Ala-Asp) box polypeptide 58 | 108.3 | 225.9 | 2.09 | 1.0E−07 |
| 209841_s_at | 180 | LRRN3 | leucine rich repeat neuronal 3 | 126.4 | 263.8 | 2.09 | 1.5E−04 |
| 225178_at | 181 | TTC14 | tetratricopeptide repeat domain 14 | 110.9 | 230.7 | 2.08 | <1.0E−07 |
| 207983_s_at | 182 | STAG2 | stromal antigen 2 | 145.6 | 302.2 | 2.08 | <1.0E−07 |
| 201027_s_at | 183 | EIF5B | eukaryotic translation initiation factor 5B | 109.8 | 227.7 | 2.07 | <1.0E−07 |
| 226643_s_at | 184 | LOC134492 | NudC domain containing 2 | 35.5 | 73.4 | 2.07 | 1.0E−07 |
| 227636_at | 185 | THAP5 | THAP domain containing 5 | 118.6 | 244.4 | 2.06 | <1.0E−07 |
| 200988_s_at | 186 | PSME3 | proteasome (prosome, macropain) activator subunit 3 (PA28 | 76.2 | 156.5 | 2.05 | 3.0E−07 |
| | | | gamma; Ki) | | | | |
| 228974_at | 187 | MGC48625 | Zinc finger protein 677 | 282.4 | 578.0 | 2.05 | 1.3E−06 |
| 228694_at | 188 | | Homo sapiens, clone IMAGE: 3352913, mRNA | 79.7 | 163.0 | 2.05 | <1.0E−07 |
| 235310_at | 189 | GCET2 | germinal center expressed transcript 2 | 127.2 | 260.3 | 2.05 | 2.3E−06 |
| 226107_at | 190 | C1GALT1 | Core 1 synthase, glycoprotein-N-acetylgalactosamine 3-beta- | 175.1 | 356.1 | 2.03 | 3.0E−07 |
| | | | galactosyltransferase, 1 | | | | |
| 224827_at | 191 | DC-UbP | Dendritic cell-derived ubiquitin-like protein | 44.5 | 90.5 | 2.03 | 7.0E−07 |
| 236321_at | 192 | LOC285550 | hypothetical protein LOC285550 | 53.9 | 109.4 | 2.03 | 3.3E−06 |
| 210073_at | 193 | ST8SIA1 | ST8 alpha-N-acetyl-neuraminide alpha-2,8-sialyltransferase 1 | 61.6 | 125.1 | 2.03 | 3.0E−06 |
| 201503_at | 194 | G3BP | Ras-GTPase-activating protein SH3-domain-binding protein | 135.7 | 273.5 | 2.02 | 4.0E−07 |
| 236583_at | 195 | | Transcribed locus | 1130.7 | 2278.6 | 2.02 | 4.0E−07 |
| 222691_at | 196 | SLC35B3 | solute carrier family 35, member B3 | 164.9 | 332.3 | 2.01 | 3.7E−06 |
| 229797_at | 197 | MCOLN3 | mucolipin 3 | 92.8 | 187.0 | 2.01 | 5.0E−05 |
| 236046_at | 198 | FLJ44896 | FLJ44896 protein | 24.4 | 48.9 | 2.01 | 1.8E−03 |
| 235424_at | 199 | C9orf42 | Chromosome 9 open reading frame 42 | 113.5 | 227.4 | 2.00 | 3.0E−07 |
| 227665_at | 200 | MCART1 | Mitochondrial carrier triple repeat 1 | 126.4 | 252.5 | 2.00 | <1.0E−07 |
| 34031_i_at | 201 | KRIT1 | KRIT1, ankyrin repeat containing | 90.4 | 180.6 | 2.00 | 9.3E−04 |
| 208864_s_at | 202 | TXN | thioredoxin | 301.9 | 602.4 | 2.00 | 1.0E−07 |
| 203404_at | 203 | ARMCX2 | armadillo repeat containing, X-linked 2 | 36.3 | 72.5 | 2.00 | 4.6E−04 |
| 201660_at | 204 | ACSL3 | Acyl-CoA synthetase long-chain family member 3 | 156.2 | 311.4 | 1.99 | <1.0E−07 |
| 203275_at | 205 | IRF2 | interferon regulatory factor 2 | 160.0 | 318.6 | 1.99 | 1.4E−05 |
| 230036_at | 206 | SAMD9L | sterile alpha motif domain containing 9-like | 372.7 | 741.5 | 1.99 | 3.0E−07 |
| 212771_at | 207 | C10orf38 | chromosome 10 open reading frame 38 | 132.6 | 263.8 | 1.99 | <1.0E−07 |
| 232614_at | 208 | BCL2 | B-cell CLL/lymphoma 2 | 307.7 | 608.3 | 1.98 | <1.0E−07 |
| 238923_at | 209 | SPOP | speckle-type POZ protein | 28.7 | 56.7 | 1.97 | 2.4E−06 |
| 239655_at | 210 | PRDM2 | PR domain containing 2, with ZNF domain | 54.8 | 108.0 | 1.97 | <1.0E−07 |
| 235177_at | 211 | LOC151194 | similar to hepatocellular carcinoma-associated antigen | 55.9 | 109.8 | 1.97 | 6.8E−06 |
| | | | HCA557b | | | | |
| 201964_at | 212 | ALS4 | amyotrophic lateral sclerosis 4 | 474.4 | 931.8 | 1.96 | 5.4E−06 |
| 201831_s_at | 213 | VDP | vesicle docking protein p115 | 83.2 | 163.5 | 1.96 | 9.9E−06 |
| 208635_x_at | 214 | NACA | nascent-polypeptide-associated complex alpha polypeptide | 4172.6 | 8189.6 | 1.96 | <1.0E−07 |
| 238554_at | 215 | LOC283852 | hypothetical protein LOC283852 | 56.1 | 109.7 | 1.96 | <1.0E−07 |
| 205078_at | 216 | PIGF | phosphatidylinositol glycan, class F | 39.9 | 78.0 | 1.95 | 2.0E−07 |
| 200033_at | 217 | DDX5 | DEAD (Asp-Glu-Ala-Asp) box polypeptide 5 | 3291.1 | 6424.5 | 1.95 | 1.0E−07 |
| 222679_s_at | 218 | DCUN1D1 | DCN1, defective in cullin neddylation 1, domain containing 1 | 28.8 | 56.2 | 1.95 | <1.0E−07 |
| 227492_at | 219 | | Transcribed locus, moderately similar to NP_689672.2 | 110.3 | 214.3 | 1.94 | 6.5E−04 |
| 200735_x_at | 220 | NACA | nascent-polypeptide-associated complex alpha polypeptide | 4369.0 | 8482.3 | 1.94 | <1.0E−07 |
| 212762_s_at | 221 | TCF7L2 | transcription factor 7-like 2 (T-cell specific, HMG-box) | 61.3 | 118.8 | 1.94 | 2.2E−04 |
| 235940_at | 222 | C9orf64 | chromosome 9 open reading frame 64 | 79.8 | 154.2 | 1.93 | 1.2E−03 |
| 224953_at | 223 | YIPF5 | Yip1 domain family, member 5 | 53.7 | 103.6 | 1.93 | 2.3E−06 |
| 225060_at | 224 | LRP11 | low density lipoprotein receptor-related protein 11 | 61.9 | 119.3 | 1.93 | 5.1E−06 |
| 221895_at | 225 | MOSPD2 | motile sperm domain containing 2 | 134.9 | 260.0 | 1.93 | 2.4E−06 |
| 236328_at | 226 | ZNF285 | zinc finger protein 285 | 60.6 | 116.3 | 1.92 | 3.3E−05 |
| 205668_at | 227 | LY75 | lymphocyte antigen 75 | 229.1 | 439.0 | 1.92 | 2.0E−05 |
| 224797_at | 228 | ARRDC3 | arrestin domain containing 3 | 319.9 | 612.9 | 1.92 | <1.0E−07 |
| 230168_at | 229 | | Transcribed locus | 198.1 | 379.3 | 1.91 | 2.9E−06 |
| 222457_s_at | 230 | EPLIN | epithelial protein lost in neoplasm beta | 41.3 | 78.9 | 1.91 | 3.6E−05 |
| 201455_s_at | 231 | NPEPPS | aminopeptidase puromycin sensitive | 131.3 | 250.7 | 1.91 | 2.7E−05 |
| 208860_s_at | 232 | ATRX | alpha thalassemia/mental retardation syndrome X-linked | 275.7 | 524.8 | 1.90 | 1.4E−02 |
| | | | (RAD54 homolog) | | | | |
| 226423_at | 233 | PAQR8 | progestin and adipoQ receptor family member VIII | 264.0 | 502.3 | 1.90 | 3.2E−06 |
| 240721_at | 234 | KIAA1967 | KIAA1967 | 32.8 | 62.2 | 1.90 | 1.1E−04 |
| 203608_at | 235 | ALDH5A1 | aldehyde dehydrogenase 5 family, member A1 | 67.4 | 127.9 | 1.90 | 1.1E−06 |
| 221916_at | 236 | NEFL | Neurofilament, light polypeptide 68 kDa | 104.2 | 196.8 | 1.89 | 7.4E−05 |
| 213246_at | 237 | C14orf109 | chromosome 14 open reading frame 109 | 168.1 | 317.4 | 1.89 | 8.1E−05 |
| 204759_at | 238 | RCBTB2 | regulator of chromosome condensation (RCC1) and BTB | 547.2 | 1033.0 | 1.89 | 2.0E−07 |
| | | | (POZ) domain containing protein 2 | | | | |
| 218984_at | 239 | FLJ20485 | hypothetical protein FLJ20485 | 250.3 | 472.2 | 1.89 | 1.2E−06 |
| 229285_at | 240 | RNASEL | ribonuclease L (2′,5′-oligoisoadenylate synthetase- | 139.9 | 263.5 | 1.88 | 1.3E−05 |
| | | | dependent) | | | | |
| 202303_x_at | 241 | SMARCA5 | SWI/SNF related, matrix associated, actin dependent | 81.2 | 153.0 | 1.88 | 7.3E−05 |
| | | | regulator of chromatin, subfamily a, member 5 | | | | |
| 216231_s_at | 242 | B2M | beta-2-microglobulin | 2873.8 | 5397.4 | 1.88 | 2.0E−05 |
| 231964_at | 243 | | MRNA; cDNA DKFZp564H1663 (from clone | 33.3 | 62.4 | 1.87 | 9.6E−05 |
| | | | DKFZp564H1663) | | | | |
| 1552790_a_at | 244 | TLOC1 | translocation protein 1 | 155.3 | 289.1 | 1.86 | 6.2E−06 |
| 238761_at | 245 | MED28 | Mediator of RNA polymerase II transcription, subunit 28 | 76.4 | 141.2 | 1.85 | 3.3E−06 |
| | | | homolog (yeast) | | | | |
| 215388_s_at | 246 | CFH /// CFHL1 | complement factor H /// complement factor H-related 1 | 45.4 | 83.8 | 1.84 | 1.1E−05 |
| 242363_at | 247 | DNCI2 | Dynein, cytoplasmic, intermediate polypeptide 2 | 116.9 | 215.6 | 1.84 | 8.5E−06 |
| 201928_at | 248 | PKP4 | plakophilin 4 | 87.7 | 161.6 | 1.84 | 1.1E−05 |
| 241497_at | 249 | | | 152.3 | 280.3 | 1.84 | 1.9E−02 |
| 1553508_at | 250 | MDS2 | myelodysplastic syndrome 2 translocation associated | 223.0 | 410.3 | 1.84 | 2.5E−04 |
| 205480_s_at | 251 | UGP2 | UDP-glucose pyrophosphorylase 2 | 568.7 | 1045.9 | 1.84 | 1.0E−06 |
| 218197_s_at | 252 | OXR1 | oxidation resistance 1 | 103.2 | 189.4 | 1.84 | <1.0E−07 |
| 242245_at | 253 | FLJ13815 | Synapse defective 1, Rho GTPase, homolog 2 (C. elegans) | 80.3 | 147.2 | 1.83 | 7.4E−06 |
| 244035_at | 254 | BCL2 | B-cell CLL/lymphoma 2 | 227.5 | 416.8 | 1.83 | 4.5E−05 |
| 212407_at | 255 | KIAA0859 | KIAA0859 | 53.4 | 97.6 | 1.83 | 1.2E−05 |
| 225290_at | 256 | | MRNA; cDNA DKFZp566C034 (from clone | 123.6 | 226.1 | 1.83 | 2.8E−06 |
| | | | DKFZp566C034) | | | | |
| 227266_s_at | 257 | FYB | FYN binding protein (FYB-120/130) | 762.5 | 1393.5 | 1.83 | 1.7E−06 |
| 214059_at | 258 | IFI44 | Interferon-induced protein 44 | 102.1 | 185.0 | 1.81 | 3.3E−04 |
| 224802_at | 259 | NDFIP2 | Nedd4 family interacting protein 2 | 66.5 | 119.9 | 1.80 | 4.9E−05 |
| 213388_at | 260 | PDE4DIP | phosphodiesterase 4D interacting protein (myomegalin) | 97.0 | 173.9 | 1.79 | 5.2E−06 |
| 243764_at | 261 | VSIG1 | V-set and immunoglobulin domain containing 1 | 128.3 | 230.0 | 1.79 | 8.4E−04 |
| 225348_at | 262 | | | 75.7 | 135.6 | 1.79 | 3.1E−05 |
| 202546_at | 263 | VAMP8 | vesicle-associated membrane protein 8 (endobrevin) | 857.9 | 1535.9 | 1.79 | 3.0E−05 |
| 230261_at | 264 | ST8SIA4 | ST8 alpha-N-acetyl-neuraminide alpha-2,8-sialyltransferase 4 | 46.0 | 82.0 | 1.78 | 5.8E−06 |
| 225669_at | 265 | IFNAR1 | interferon (alpha, beta and omega) receptor 1 | 137.2 | 243.1 | 1.77 | 1.2E−05 |
| 223377_x_at | 266 | CISH | cytokine inducible SH2-containing protein | 215.3 | 381.1 | 1.77 | 2.5E−04 |
| 203465_at | 267 | MRPL19 | mitochondrial ribosomal protein L19 | 46.6 | 82.3 | 1.77 | 1.6E−06 |
| 229090_at | 268 | LOC220930 | hypothetical protein LOC220930 | 38.1 | 66.6 | 1.75 | 1.4E−05 |
| 244042_x_at | 269 | | Transcribed locus | 22.2 | 38.6 | 1.74 | 1.5E−03 |
| 235551_at | 270 | WDR4 | WD repeat domain 4 | 35.8 | 62.3 | 1.74 | 2.4E−03 |
| 232584_at | 271 | C20orf17 | Chromosome 20 open reading frame 17 | 92.8 | 159.8 | 1.72 | 2.8E−05 |
| 221805_at | 272 | NEFL | neurofilament, light polypeptide 68 kDa | 15.7 | 26.8 | 1.71 | 1.2E−04 |
| 1559025_at | 273 | SEPT9 | septin 9 | 35.1 | 59.1 | 1.69 | 4.8E−04 |
| 213808_at | 274 | ADAM23 | ADAM metallopeptidase domain 23 | 46.7 | 78.7 | 1.68 | 8.7E−05 |
| 212063_at | 275 | CD44 | CD44 antigen (homing function and Indian blood group | 998.6 | 1680.2 | 1.68 | 5.0E−07 |
| | | | system) | | | | |
| 235422_at | 276 | | LOC440461 | 158.5 | 264.1 | 1.67 | 3.9E−03 |
| 208778_s_at | 277 | TCP1 | t-complex 1 | 1060.4 | 1760.8 | 1.66 | 6.0E−05 |
| 229850_at | 278 | FVT1 | Follicular lymphoma variant translocation 1 | 32.5 | 53.0 | 1.63 | 1.1E−03 |
| 205898_at | 279 | CX3CR1 | chemokine (C—X3—C motif) receptor 1 | 1377.8 | 2218.7 | 1.61 | 5.0E−03 |
| 1558569_at | 280 | MAML2 | Mastermind-like 2 (Drosophila) | 406.9 | 651.7 | 1.60 | 7.1E−03 |
| 214683_s_at | 281 | CLK1 | CDC-like kinase 1 | 510.2 | 805.3 | 1.58 | 1.0E−05 |
| 221223_x_at | 282 | CISH | cytokine inducible SH2-containing protein | 235.2 | 369.2 | 1.57 | 6.1E−04 |
| 239944_at | 283 | | CDNA FLJ42561 fis, clone BRACE3006463 | 102.3 | 160.4 | 1.57 | 4.8E−04 |
| 226671_at | 284 | | CDNA clone IMAGE: 4797120 | 75.0 | 117.6 | 1.57 | 6.9E−05 |
| 211985_s_at | 285 | CALM1 | calmodulin 1 (phosphorylase kinase, delta) | 720.8 | 1118.7 | 1.55 | 5.0E−03 |
| 209993_at | 286 | ABCB1 | ATP-binding cassette, sub-family B (MDR/TAP), member 1 | 53.2 | 82.4 | 1.55 | 1.3E−03 |
| 207238_s_at | 287 | PTPRC | protein tyrosine phosphatase, receptor type, C | 245.1 | 362.9 | 1.48 | 3.0E−05 |
| 214741_at | 288 | ZNF131 | zinc finger protein 131 (clone pHZ-10) | 136.6 | 195.6 | 1.43 | 8.2E−03 |
| 204042_at | 289 | WASF3 | WAS protein family, member 3 | 138.3 | 146.8 | 1.06 | 2.5E−02 |
| 202652_at | 290 | APBB1 | amyloid beta (A4) precursor protein-binding, family B, | 207.2 | 186.8 | −1.11 | 2.9E−02 |
| | | | member 1 (Fe65) | | | | |
| 201005_at | 291 | CD9 | CD9 antigen (p24) | 443.6 | 384.1 | −1.15 | 3.0E−03 |
| 212834_at | 292 | DDX52 | DEAD (Asp-Glu-Ala-Asp) box polypeptide 52 | 273.1 | 231.5 | −1.18 | 2.8E−02 |
| 220496_at | 293 | CLEC1B | C-type lectin domain family 1, member B | 343.0 | 290.4 | −1.18 | 1.2E−02 |
| 222891_s_at | 294 | BCL11A | B-cell CLL/lymphoma 11A (zinc finger protein) | 84.4 | 71.3 | −1.18 | 3.4E−02 |
| 235490_at | 295 | MGC10744 | hypothetical protein MGC10744 | 82.4 | 68.0 | −1.21 | 1.1E−02 |
| 215894_at | 296 | PTGDR | prostaglandin D2 receptor (DP) | 941.7 | 770.9 | −1.22 | 2.8E−02 |
| 240077_at | 297 | C18orf1 | Chromosome 18 open reading frame 1 | 236.8 | 193.5 | −1.22 | 3.3E−03 |
| 225247_at | 298 | C19orf6 | chromosome 19 open reading frame 6 | 937.6 | 763.7 | −1.23 | 1.4E−02 |
| 202729_s_at | 299 | LTBP1 | latent transforming growth factor beta binding protein 1 | 159.6 | 129.0 | −1.24 | 2.7E−02 |
| 233350_s_at | 300 | TEX264 | testis expressed sequence 264 | 266.1 | 215.0 | −1.24 | 6.6E−03 |
| 1556338_at | 301 | UBE1C | Ubiquitin-activating enzyme E1C (UBA3 homolog, yeast) | 85.6 | 69.1 | −1.24 | 2.8E−02 |
| 244026_at | 302 | ELL2 | Elongation factor, RNA polymerase II, 2 | 66.0 | 53.1 | −1.24 | 6.7E−03 |
| 202932_at | 303 | YES1 | v-yes-1 Yamaguchi sarcoma viral oncogene homolog 1 | 302.8 | 237.2 | −1.28 | 1.9E−02 |
| 201163_s_at | 304 | IGFBP7 | insulin-like growth factor binding protein 7 | 353.6 | 275.9 | −1.28 | 2.2E−02 |
| 212081_x_at | 305 | BAT2 | HLA-B associated transcript 2 | 272.0 | 212.2 | −1.28 | 1.6E−03 |
| 243006_at | 306 | FYN | FYN oncogene related to SRC, FGR, YES | 605.3 | 470.5 | −1.29 | 2.3E−02 |
| 224376_s_at | 307 | C20orf24 | chromosome 20 open reading frame 24 | 2071.4 | 1608.8 | −1.29 | 2.0E−02 |
| 240038_at | 308 | ELL2 | Elongation factor, RNA polymerase II, 2 | 380.3 | 295.2 | −1.29 | 1.2E−03 |
| 224496_s_at | 309 | MGC10744 | hypothetical protein MGC10744 | 368.1 | 285.3 | −1.29 | 5.2E−03 |
| 222687_s_at | 310 | PHCA | phytoceramidase, alkaline | 66.7 | 51.7 | −1.29 | 2.2E−02 |
| 204443_at | 311 | ARSA | arylsulfatase A | 142.9 | 110.3 | −1.30 | 4.7E−03 |
| 215813_s_at | 312 | PTGS1 | prostaglandin-endoperoxide synthase 1 | 287.2 | 221.5 | −1.30 | 2.6E−03 |
| 219090_at | 313 | SLC24A3 | solute carrier family 24 (sodium/potassium/calcium | 110.5 | 85.1 | −1.30 | 2.9E−02 |
| | | | exchanger), member 3 | | | | |
| 204232_at | 314 | FCER1G | Fc fragment of IgE, high affinity I, receptor for; gamma | 727.1 | 559.0 | −1.30 | 1.3E−02 |
| | | | polypeptide | | | | |
| 208777_s_at | 315 | PSMD11 | proteasome (prosome, macropain) 26S subunit, non-ATPase, | 595.3 | 457.5 | −1.30 | 2.8E−02 |
| | | | 11 | | | | |
| 241985_at | 316 | JMY | junction-mediating and regulatory protein | 495.0 | 379.9 | −1.30 | 1.8E−03 |
| 233127_at | 317 | ZNF331 | Zinc finger protein 331 | 246.6 | 188.2 | −1.31 | 4.8E−03 |
| 1552628_a_at | 318 | FLJ22313 | hypothetical protein FLJ22313 | 214.5 | 163.1 | −1.32 | 1.9E−02 |
| 201466_s_at | 319 | JUN | v-jun sarcoma virus 17 oncogene homolog (avian) | 1391.1 | 1057.4 | −1.32 | 4.4E−03 |
| 213183_s_at | 320 | CDKN1C | Cyclin-dependent kinase inhibitor 1C (p57, Kip2) | 52.7 | 40.0 | −1.32 | 1.7E−03 |
| 209305_s_at | 321 | GADD45B | growth arrest and DNA-damage-inducible, beta | 146.7 | 110.9 | −1.32 | 3.1E−02 |
| 209301_at | 322 | CA2 | carbonic anhydrase II | 167.0 | 126.1 | −1.32 | 1.6E−02 |
| 212062_at | 323 | ATP9A | ATPase, Class II, type 9A | 159.3 | 120.3 | −1.32 | 6.4E−03 |
| 227647_at | 324 | KCNE3 | potassium voltage-gated channel, Isk-related family, member 3 | 117.0 | 88.1 | −1.33 | 4.0E−03 |
| 209199_s_at | 325 | MEF2C | MADS box transcription enhancer factor 2, polypeptide C | 482.1 | 362.9 | −1.33 | 2.5E−03 |
| | | | (myocyte enhancer factor 2C) | | | | |
| 200869_at | 326 | RPL18A | ribosomal protein L18a | 1154.0 | 859.9 | −1.34 | 1.0E−02 |
| 208931_s_at | 327 | ILF3 | interleukin enhancer binding factor 3, 90 kDa | 362.4 | 269.0 | −1.35 | 2.6E−03 |
| 223204_at | 328 | DKFZp434L142 | hypothetical protein DKFZp434L142 | 83.7 | 61.7 | −1.36 | 2.4E−02 |
| 201278_at | 329 | DAB2 | Disabled homolog 2, mitogen-responsive phosphoprotein | 127.5 | 94.0 | −1.36 | 2.6E−03 |
| 231225_at | 330 | | | 116.3 | 85.6 | −1.36 | 2.3E−03 |
| 204141_at | 331 | TUBB2 | tubulin, beta 2 | 1463.0 | 1068.9 | −1.37 | 1.2E−03 |
| 230645_at | 332 | FRMD3 | FERM domain containing 3 | 167.9 | 122.2 | −1.37 | 1.8E−02 |
| 205859_at | 333 | LY86 | lymphocyte antigen 86 | 346.9 | 251.9 | −1.38 | 1.3E−02 |
| 243618_s_at | 334 | LOC152485 | Hypothetical protein LOC152485 | 273.7 | 198.2 | −1.38 | 9.5E−05 |
| 203827_at | 335 | WIPI49 | WD40 repeat protein Interacting with phosphoInositides of | 97.7 | 70.3 | −1.39 | 6.7E−03 |
| | | | 49 kDa | | | | |
| 227798_at | 336 | SMAD1 | SMAD, mothers against DPP homolog 1 (Drosophila) | 43.5 | 31.3 | −1.39 | 1.2E−02 |
| 208893_s_at | 337 | DUSP6 | dual specificity phosphatase 6 | 147.1 | 105.1 | −1.40 | 3.3E−03 |
| 241702_at | 338 | HNRPD | Heterogeneous nuclear ribonucleoprotein D | 327.6 | 233.3 | −1.40 | 5.7E−04 |
| 210836_x_at | 339 | PDE4D | phosphodiesterase 4D | 131.6 | 93.7 | −1.40 | 4.3E−05 |
| 229560_at | 340 | TLR8 | toll-like receptor 8 | 112.6 | 80.0 | −1.41 | 6.8E−03 |
| 209162_s_at | 341 | PRPF4 | PRP4 pre-mRNA processing factor 4 homolog (yeast) | 57.5 | 40.9 | −1.41 | 7.6E−03 |
| 228771_at | 342 | ADRBK2 | adrenergic, beta, receptor kinase 2 | 467.0 | 330.1 | −1.41 | 1.1E−03 |
| 227146_at | 343 | QSCN6L1 | quiescin Q6-like 1 | 1040.2 | 735.0 | −1.42 | 2.0E−03 |
| 216035_x_at | 344 | TCF7L2 | transcription factor 7-like 2 (T-cell specific, HMG-box) | 211.6 | 149.5 | −1.42 | 5.3E−03 |
| 207840_at | 345 | CD160 | CD160 antigen | 594.9 | 418.4 | −1.42 | 1.9E−03 |
| 203603_s_at | 346 | ZFHX1B | zinc finger homeobox 1b | 224.6 | 157.8 | −1.42 | 1.4E−03 |
| 215342_s_at | 347 | RABGAP1L | RAB GTPase activating protein 1-like | 66.4 | 46.6 | −1.42 | 7.4E−03 |
| 228638_at | 348 | MGC34648 | Family with sequence similarity 76, member A | 189.6 | 133.1 | −1.42 | 2.4E−03 |
| 230134_s_at | 349 | MNAB | membrane associated DNA binding protein | 559.5 | 392.7 | −1.42 | 1.5E−05 |
| 220439_at | 350 | RIN3 | Ras and Rab interactor 3 | 104.5 | 73.2 | −1.43 | 5.4E−04 |
| 1559739_at | 351 | CHPT1 | Choline phosphotransferase 1 | 76.2 | 53.2 | −1.43 | 9.7E−04 |
| 223650_s_at | 352 | NRBF2 | nuclear receptor binding factor 2 | 72.7 | 50.8 | −1.43 | 1.3E−02 |
| 226982_at | 353 | ELL2 | elongation factor, RNA polymerase II, 2 | 462.1 | 321.9 | −1.44 | 2.5E−03 |
| 226763_at | 354 | SESTD1 | SEC14 and spectrin domains 1 | 100.9 | 70.3 | −1.44 | 6.1E−04 |
| 1555890_at | 355 | OR2A20P | Olfactory receptor, family 2, subfamily A, member 20 | 78.4 | 54.6 | −1.44 | 1.4E−02 |
| | | | pseudogene | | | | |
| 208703_s_at | 356 | APLP2 | amyloid beta (A4) precursor-like protein 2 | 303.4 | 209.5 | −1.45 | 1.2E−03 |
| 226841_at | 357 | MPEG1 | macrophage expressed gene 1 | 322.0 | 221.7 | −1.45 | 4.6E−03 |
| 220005_at | 358 | P2RY13 | purinergic receptor P2Y, G-protein coupled, 13 | 40.9 | 28.2 | −1.45 | 1.9E−02 |
| 206036_s_at | 359 | REL | v-rel reticuloendotheliosis viral oncogene homolog (avian) | 126.1 | 86.7 | −1.45 | 7.5E−04 |
| 202437_s_at | 360 | CYP1B1 | cytochrome P450, family 1, subfamily B, polypeptide 1 | 32.2 | 22.1 | −1.46 | 2.8E−03 |
| 202933_s_at | 361 | YES1 | v-yes-1 Yamaguchi sarcoma viral oncogene homolog 1 | 543.7 | 372.9 | −1.46 | 2.3E−04 |
| 208892_s_at | 362 | DUSP6 | dual specificity phosphatase 6 | 223.4 | 153.1 | −1.46 | 9.6E−04 |
| 213566_at | 363 | RNASE6 | ribonuclease, RNase A family, k6 /// ribonuclease, RNase A | 213.5 | 146.0 | −1.46 | 9.3E−03 |
| | | | family, k6 | | | | |
| 221731_x_at | 364 | CSPG2 | chondroitin sulfate proteoglycan 2 (versican) | 1677.8 | 1145.3 | −1.47 | 3.4E−03 |
| 1558739_at | 365 | DUSP16 | Dual specificity phosphatase 16 | 114.0 | 77.6 | −1.47 | 1.9E−03 |
| 226865_at | 366 | | MRNA; cDNA DKFZp564O0862 (from clone | 96.4 | 65.6 | −1.47 | 2.2E−03 |
| | | | DKFZp564O0862) | | | | |
| 221676_s_at | 367 | CORO1C | coronin, actin binding protein, 1C | 122.1 | 83.1 | −1.47 | 1.7E−04 |
| 216037_x_at | 368 | TCF7L2 | transcription factor 7-like 2 (T-cell specific, HMG-box) | 143.2 | 97.4 | −1.47 | 1.7E−03 |
| 207550_at | 369 | MPL | myeloproliferative leukemia virus oncogene | 66.7 | 45.3 | −1.47 | 2.0E−03 |
| 209967_s_at | 370 | CREM | cAMP responsive element modulator | 1515.2 | 1029.7 | −1.47 | 7.8E−04 |
| 210837_s_at | 371 | PDE4D | phosphodiesterase 4D, cAMP-specific | 123.2 | 83.7 | −1.47 | 6.9E−05 |
| 243683_at | 372 | MORF4L2 | Mortality factor 4 like 2 | 80.6 | 54.4 | −1.48 | 1.8E−02 |
| 204834_at | 373 | FGL2 | fibrinogen-like 2 | 211.1 | 141.9 | −1.49 | 5.0E−03 |
| 240221_at | 374 | CSNK1A1 | Casein kinase 1, alpha 1 | 281.5 | 189.1 | −1.49 | 3.7E−04 |
| 240024_at | 375 | SEC14L2 | SEC14-like 2 (S. cerevisiae) | 164.1 | 110.1 | −1.49 | 1.3E−04 |
| 205789_at | 376 | CD1D | CD1D antigen, d polypeptide | 51.7 | 34.7 | −1.49 | 7.6E−03 |
| 235592_at | 377 | ELL2 | Elongation factor, RNA polymerase II, 2 | 273.5 | 182.8 | −1.50 | 6.5E−04 |
| 215440_s_at | 378 | BEXL1 | brain expressed X-linked-like 1 | 515.3 | 344.2 | −1.50 | 3.7E−03 |
| 226152_at | 379 | TTC7B | tetratricopeptide repeat domain 7B | 47.3 | 31.6 | −1.50 | 1.9E−02 |
| 216202_s_at | 380 | SPTLC2 | serine palmitoyltransferase, long chain base subunit 2 | 72.6 | 48.4 | −1.50 | 2.5E−03 |
| 223940_x_at | 381 | MALAT1 | metastasis associated lung adenocarcinoma transcript 1 (non- | 317.3 | 211.3 | −1.50 | 1.6E−03 |
| | | | coding RNA) | | | | |
| 219667_s_at | 382 | BANK1 | B-cell scaffold protein with ankyrin repeats 1 | 186.1 | 123.6 | −1.51 | 2.8E−04 |
| 217805_at | 383 | ILF3 | interleukin enhancer binding factor 3, 90 kDa | 200.4 | 133.1 | −1.51 | 2.0E−03 |
| 204620_s_at | 384 | CSPG2 | chondroitin sulfate proteoglycan 2 (versican) | 466.9 | 309.9 | −1.51 | 2.4E−03 |
| 205686_s_at | 385 | CD86 | CD86 antigen (CD28 antigen ligand 2, B7-2 antigen) | 78.0 | 51.6 | −1.51 | 7.1E−04 |
| 212651_at | 386 | RHOBTB1 | Rho-related BTB domain containing 1 | 33.1 | 21.9 | −1.51 | 3.2E−03 |
| 232027_at | 387 | SYNE1 | spectrin repeat containing, nuclear envelope 1 | 186.1 | 122.7 | −1.52 | 3.6E−04 |
| 233614_at | 388 | CBLB | Cas-Br-M (murine) ecotropic retroviral transforming | 91.8 | 60.4 | −1.52 | 3.0E−04 |
| | | | sequence b | | | | |
| 203392_s_at | 389 | CTBP1 | C-terminal binding protein 1 | 5636.1 | 3710.4 | −1.52 | 2.5E−03 |
| 229128_s_at | 390 | ANP32E | Acidic (leucine-rich) nuclear phosphoprotein 32 family, | 69.0 | 45.4 | −1.52 | 6.8E−03 |
| | | | member E | | | | |
| 226818_at | 391 | MPEG1 | macrophage expressed gene 1 | 402.1 | 263.0 | −1.53 | 1.0E−03 |
| 223343_at | 392 | MS4A7 | membrane-spanning 4-domains, subfamily A, member 7 | 410.6 | 268.1 | −1.53 | 7.1E−03 |
| 210146_x_at | 393 | LILRB2 | leukocyte immunoglobulin-like receptor, subfamily B, | 42.7 | 27.8 | −1.53 | 3.0E−03 |
| | | | member 2 | | | | |
| 203817_at | 394 | GUCY1B3 | guanylate cyclase 1, soluble, beta 3 | 168.0 | 109.4 | −1.54 | 1.8E−03 |
| 1553681_a_at | 395 | PRF1 | perforin 1 (pore forming protein) | 234.0 | 152.2 | −1.54 | 3.1E−04 |
| 243296_at | 396 | PBEF1 | Pre-B-cell colony enhancing factor 1 | 351.7 | 228.7 | −1.54 | 4.6E−03 |
| 208146_s_at | 397 | CPVL | carboxypeptidase, vitellogenic-like | 159.4 | 103.1 | −1.55 | 7.3E−03 |
| 225567_at | 398 | | Hypothetical LOC388114 | 131.4 | 85.0 | −1.55 | 1.7E−03 |
| 201635_s_at | 399 | FXR1 | fragile X mental retardation, autosomal homolog 1 | 157.3 | 101.5 | −1.55 | 8.3E−05 |
| 223922_x_at | 400 | MS4A6A | membrane-spanning 4-domains, subfamily A, member 6A | 241.4 | 155.5 | −1.55 | 7.7E−04 |
| 206488_s_at | 401 | CD36 | CD36 antigen (collagen type I receptor, thrombospondin | 48.2 | 31.0 | −1.55 | 6.0E−03 |
| | | | receptor) | | | | |
| 203799_at | 402 | CD302 | CD302 antigen | 185.9 | 119.6 | −1.55 | 3.7E−03 |
| 211676_s_at | 403 | IFNGR1 | interferon gamma receptor 1 | 194.6 | 125.2 | −1.55 | 3.0E−04 |
| 207795_s_at | 404 | KLRD1 | killer cell lectin-like receptor subfamily D, member 1 | 408.3 | 262.4 | −1.56 | 1.3E−03 |
| 203922_s_at | 405 | CYBB | cytochrome b-245, beta polypeptide (chronic granulomatous | 129.1 | 82.9 | −1.56 | 1.0E−03 |
| | | | disease) | | | | |
| 211397_x_at | 406 | KIR2DL2 | killer cell immunoglobulin-like receptor, two domains, long | 134.9 | 86.6 | −1.56 | 3.2E−04 |
| | | | cytoplasmic tail, 2 | | | | |
| 207540_s_at | 407 | SYK | spleen tyrosine kinase | 612.6 | 392.7 | −1.56 | 1.9E−04 |
| 228030_at | 408 | RBM6 | RNA binding motif protein 6 | 68.5 | 43.8 | −1.56 | 9.8E−03 |
| 1553704_x_at | 409 | | CDNA FLJ13242 fis, clone OVARC1000578 | 402.5 | 257.4 | −1.56 | 5.8E−06 |
| 1558710_at | 410 | ARIH1 | Ariadne homolog, ubiquitin-conjugating enzyme E2 binding | 378.3 | 241.8 | −1.56 | 2.6E−03 |
| | | | protein, 1 | | | | |
| 1552398_a_at | 411 | CLEC12A | C-type lectin domain family 12, member A | 309.8 | 198.0 | −1.56 | 1.7E−03 |
| 217739_s_at | 412 | PBEF1 | pre-B-cell colony enhancing factor 1 | 700.6 | 447.6 | −1.57 | 1.2E−03 |
| 241403_at | 413 | CLK4 | CDC-like kinase 4 | 101.4 | 64.7 | −1.57 | 1.2E−03 |
| 225782_at | 414 | MSRB3 | methionine sulfoxide reductase B3 | 62.4 | 39.8 | −1.57 | 6.2E−04 |
| 209883_at | 415 | GLT25D2 | glycosyltransferase 25 domain containing 2 | 112.0 | 71.4 | −1.57 | 1.9E−04 |
| 205997_at | 416 | ADAM28 | ADAM metallopeptidase domain 28 | 84.8 | 53.9 | −1.57 | 5.5E−05 |
| 227088_at | 417 | PDE5A | phosphodiesterase 5A, cGMP-specific | 73.1 | 46.5 | −1.57 | 2.2E−03 |
| 226489_at | 418 | TMCC3 | transmembrane and coiled-coil domain family 3 | 106.4 | 67.6 | −1.57 | 5.1E−04 |
| 220122_at | 419 | MCTP1 | multiple C2-domains with two transmembrane regions 1 | 55.1 | 35.0 | −1.58 | 1.6E−03 |
| 228049_x_at | 420 | | Transcribed locus | 463.0 | 293.5 | −1.58 | 1.5E−06 |
| 244804_at | 421 | SQSTM1 | Sequestosome 1 | 1147.8 | 726.4 | −1.58 | 2.0E−04 |
| 227889_at | 422 | FLJ20481 | hypothetical protein FLJ20481 | 39.6 | 25.0 | −1.58 | 7.5E−05 |
| 224657_at | 423 | ERRFI1 | ERBB receptor feedback inhibitor 1 | 408.7 | 257.8 | −1.59 | 1.9E−04 |
| 213830_at | 424 | TRD@ | T cell receptor delta locus | 1434.6 | 904.8 | −1.59 | 9.3E−04 |
| 1569856_at | 425 | TPP2 | tripeptidyl peptidase II | 167.2 | 105.1 | −1.59 | 4.3E−06 |
| 201218_at | 426 | CTBP2 | C-terminal binding protein 2 | 292.5 | 183.7 | −1.59 | 3.7E−04 |
| 201506_at | 427 | TGFBI | transforming growth factor, beta-induced, 68 kDa | 273.2 | 170.9 | −1.60 | 9.5E−04 |
| 210660_at | 428 | LILRA1 | leukocyte immunoglobulin-like receptor, subfamily A (with | 251.0 | 156.8 | −1.60 | 1.2E−03 |
| | | | TM domain) | | | | |
| 217764_s_at | 429 | RAB31 | RAB31, member RAS oncogene family | 472.6 | 295.1 | −1.60 | 4.3E−03 |
| 208750_s_at | 430 | ARF1 | ADP-ribosylation factor 1 | 107.2 | 66.9 | −1.60 | 5.1E−03 |
| 212382_at | 431 | TCF4 | Transcription factor 4 | 76.9 | 48.0 | −1.60 | 7.4E−06 |
| 231777_at | 432 | CSNK2B | Casein kinase 2, beta polypeptide | 81.0 | 50.5 | −1.60 | 1.1E−04 |
| 202381_at | 433 | ADAM9 | ADAM metallopeptidase domain 9 (meltrin gamma) | 85.7 | 53.2 | −1.61 | 1.6E−04 |
| 206666_at | 434 | GZMK | granzyme K (granzyme 3; tryptase II) | 1619.7 | 1004.5 | −1.61 | 6.8E−05 |
| 223344_s_at | 435 | MS4A7 | membrane-spanning 4-domains, subfamily A, member 7 | 125.1 | 77.4 | −1.61 | 2.3E−02 |
| 221841_s_at | 436 | KLF4 | Kruppel-like factor 4 (gut) | 2386.0 | 1475.8 | −1.62 | 4.0E−04 |
| 204621_s_at | 437 | NR4A2 | nuclear receptor subfamily 4, group A, member 2 | 1681.2 | 1039.7 | −1.62 | 3.5E−05 |
| 211478_s_at | 438 | DPP4 | dipeptidylpeptidase 4 (CD26, adenosine deaminase | 87.7 | 54.1 | −1.62 | 1.8E−03 |
| | | | complexing protein 2) | | | | |
| 240240_at | 439 | UBE2J2 | Ubiquitin-conjugating enzyme E2, J2 (UBC6 homolog, | 93.8 | 57.8 | −1.62 | 1.3E−05 |
| | | | yeast) | | | | |
| 204015_s_at | 440 | DUSP4 | dual specificity phosphatase 4 | 161.5 | 99.4 | −1.62 | 4.1E−05 |
| 200751_s_at | 441 | HNRPC | heterogeneous nuclear ribonucleoprotein C (C1/C2) | 48.5 | 29.7 | −1.63 | 1.9E−03 |
| 230983_at | 442 | BCNP1 | B-cell novel protein 1 | 488.5 | 298.8 | −1.63 | 3.9E−05 |
| 223125_s_at | 443 | C1orf21 | chromosome 1 open reading frame 21 | 756.2 | 461.1 | −1.64 | 6.6E−05 |
| 217762_s_at | 444 | RAB31 | RAB31, member RAS oncogene family | 554.1 | 337.8 | −1.64 | 5.8E−03 |
| 210555_s_at | 445 | NFATC3 | nuclear factor of activated T-cells, cytoplasmic, calcineurin- | 527.4 | 321.1 | −1.64 | 3.3E−04 |
| | | | dependent 3 | | | | |
| 211824_x_at | 446 | NALP1 | NACHT, leucine rich repeat and PYD (pyrin domain) | 231.4 | 140.7 | −1.64 | 1.4E−04 |
| | | | containing 1 | | | | |
| 201104_x_at | 447 | AG1 | AG1 protein | 434.2 | 264.0 | −1.64 | 9.8E−04 |
| 204285_s_at | 448 | PMAIP1 | phorbol-12-myristate-13-acetate-induced protein 1 | 1151.8 | 699.9 | −1.65 | 2.5E−04 |
| 201798_s_at | 449 | FER1L3 | fer-1-like 3, myoferlin (C. elegans) | 117.2 | 71.2 | −1.65 | 1.9E−03 |
| 210778_s_at | 450 | MXD4 | MAX dimerization protein 4 | 347.9 | 211.1 | −1.65 | 3.1E−06 |
| 208891_at | 451 | DUSP6 | dual specificity phosphatase 6 | 188.8 | 114.4 | −1.65 | 1.9E−05 |
| 208438_s_at | 452 | FGR | Gardner-Rasheed feline sarcoma viral (v-fgr) oncogene | 408.8 | 247.4 | −1.65 | 3.6E−06 |
| | | | homolog | | | | |
| 39318_at | 453 | TCL1A | T-cell leukemia/lymphoma 1A | 156.1 | 94.3 | −1.66 | 5.2E−04 |
| 211532_x_at | 454 | KIR2DS2 | killer cell immunoglobulin-like receptor, two domains, short | 212.6 | 128.1 | −1.66 | 6.7E−04 |
| | | | cytoplasmic tail, 2 | | | | |
| 226023_at | 455 | MAP2K7 | mitogen-activated protein kinase kinase 7 | 181.6 | 109.3 | −1.66 | 1.1E−05 |
| 201341_at | 456 | ENC1 | ectodermal-neural cortex (with BTB-like domain) | 797.8 | 479.7 | −1.66 | 3.0E−06 |
| 231889_at | 457 | RBAF600 | retinoblastoma-associated factor 600 | 143.4 | 86.1 | −1.67 | 1.8E−05 |
| 241762_at | 458 | | | 67.3 | 40.4 | −1.67 | 2.8E−06 |
| 206414_s_at | 459 | DDEF2 | development and differentiation enhancing factor 2 | 168.4 | 100.8 | −1.67 | 5.9E−04 |
| 209995_s_at | 460 | TCL1A | T-cell leukemia/lymphoma 1A /// T-cell leukemia/lymphoma | 262.7 | 157.1 | −1.67 | 6.5E−04 |
| | | | 1A | | | | |
| 214958_s_at | 461 | EVER1 | epidermodysplasia verruciformis 1 | 488.7 | 292.1 | −1.67 | 2.9E−04 |
| 202464_s_at | 462 | PFKFB3 | 6-phosphofructo-2-kinase/fructose-2,6-biphosphatase 3 | 1425.4 | 850.3 | −1.68 | 8.0E−06 |
| 201906_s_at | 463 | CTDSPL | carboxy-terminal domain, RNA polymerase II, polypeptide | 262.5 | 156.2 | −1.68 | 6.8E−04 |
| | | | A small phosphatase-like | | | | |
| 228204_at | 464 | PSMB4 | Proteasome (prosome, macropain) subunit, beta type, 4 | 189.6 | 112.7 | −1.68 | 4.1E−04 |
| 224576_at | 465 | KIAA1181 | endoplasmic reticulum-golgi intermediate compartment 32 kDa | 626.0 | 371.7 | −1.68 | 3.6E−06 |
| | | | protein | | | | |
| 210164_at | 466 | GZMB | granzyme B | 2606.6 | 1546.0 | −1.69 | 7.0E−07 |
| 1555117_at | 467 | | | 122.1 | 72.4 | −1.69 | 1.1E−05 |
| 243115_at | 468 | | Transcribed locus | 41.3 | 24.4 | −1.69 | 2.0E−04 |
| 225466_at | 469 | FLJ36874 | hypothetical protein FLJ36874 | 822.1 | 485.3 | −1.69 | 8.0E−06 |
| 207723_s_at | 470 | KLRC3 | killer cell lectin-like receptor subfamily C, member 3 | 194.8 | 114.9 | −1.70 | 3.9E−04 |
| 223126_s_at | 471 | C1orf21 | chromosome 1 open reading frame 21 | 172.3 | 101.6 | −1.70 | 5.0E−06 |
| 226018_at | 472 | Ells1 | hypothetical protein Ells1 | 478.6 | 280.7 | −1.70 | 2.5E−05 |
| 221293_s_at | 473 | DEF6 | differentially expressed in FDCP 6 homolog (mouse) | 158.7 | 92.9 | −1.71 | 1.7E−05 |
| 215047_at | 474 | TRIM58 | tripartite motif-containing 58 | 41.7 | 24.4 | −1.71 | 1.4E−03 |
| 206363_at | 475 | MAF | v-maf musculoaponeurotic fibrosarcoma oncogene homolog | 926.4 | 541.9 | −1.71 | 2.7E−05 |
| | | | (avian) | | | | |
| 212843_at | 476 | NCAM1 | neural cell adhesion molecule 1 | 418.3 | 244.4 | −1.71 | 7.0E−07 |
| 1555705_a_at | 477 | CKLFSF3 | chemokine-like factor superfamily 3 | 521.1 | 304.2 | −1.71 | 8.0E−07 |
| 204466_s_at | 478 | SNCA | synuclein, alpha | 281.6 | 164.1 | −1.72 | 3.5E−05 |
| 217388_s_at | 479 | KYNU | kynureninase (L-kynurenine hydrolase) | 176.1 | 102.6 | −1.72 | 1.6E−04 |
| 206283_s_at | 480 | TAL1 | T-cell acute lymphocytic leukemia 1 | 123.3 | 71.7 | −1.72 | 2.7E−04 |
| 204249_s_at | 481 | LMO2 | LIM domain only 2 (rhombotin-like 1) | 205.5 | 119.4 | −1.72 | 1.1E−04 |
| 208782_at | 482 | FSTL1 | follistatin-like 1 | 191.3 | 110.8 | −1.73 | 1.1E−04 |
| 203923_s_at | 483 | CYBB | cytochrome b-245, beta polypeptide | 246.0 | 142.2 | −1.73 | 9.2E−04 |
| 224925_at | 484 | PREX1 | phosphatidylinositol 3,4,5-trisphosphate-dependent RAC | 924.7 | 534.6 | −1.73 | 2.4E−06 |
| | | | exchanger 1 | | | | |
| 205987_at | 485 | CD1C | CD1C antigen, c polypeptide | 157.0 | 90.7 | −1.73 | 1.8E−05 |
| 205758_at | 486 | CD8A | CD8 antigen | 658.7 | 380.5 | −1.73 | 3.0E−06 |
| 1555756_a_at | 487 | CLEC7A | C-type lectin domain family 7, member A | 33.4 | 19.2 | −1.73 | 1.2E−02 |
| 202917_s_at | 488 | S100A8 | S100 calcium binding protein A8 (calgranulin A) | 4374.7 | 2519.0 | −1.74 | 1.0E−03 |
| 215275_at | 489 | TRAF3IP3 | TRAF3 interacting protein 3 | 84.9 | 48.9 | −1.74 | 1.4E−04 |
| 207156_at | 490 | HIST1H2AG | histone 1, H2ag | 120.4 | 69.2 | −1.74 | 3.4E−03 |
| 214567_s_at | 491 | XCL1 | chemokine (C motif) ligand 1 | 217.3 | 124.4 | −1.75 | 5.7E−06 |
| 222892_s_at | 492 | TMEM40 | transmembrane protein 40 | 268.7 | 153.4 | −1.75 | 9.9E−05 |
| 205128_x_at | 493 | PTGS1 | prostaglandin-endoperoxide synthase 1 | 614.8 | 350.3 | −1.76 | 1.4E−04 |
| 207163_s_at | 494 | AKT1 | v-akt murine thymoma viral oncogene homolog 1 | 243.5 | 138.6 | −1.76 | 1.2E−06 |
| 215684_s_at | 495 | ASCC2 | activating signal cointegrator 1 complex subunit 2 | 160.1 | 91.0 | −1.76 | 1.0E−05 |
| 223280_x_at | 496 | MS4A6A | membrane-spanning 4-domains, subfamily A, member 6A | 538.0 | 305.0 | −1.76 | 2.4E−04 |
| 221986_s_at | 497 | KLHL24 | kelch-like 24 (Drosophila) | 513.2 | 290.7 | −1.76 | 5.9E−05 |
| 222483_at | 498 | EFHD2 | EF-hand domain family, member D2 | 805.2 | 454.4 | −1.77 | <1.0E−07 |
| 205826_at | 499 | MYOM2 | myomesin (M-protein) 2, 165 kDa /// myomesin (M-protein) | 1350.7 | 761.8 | −1.77 | 1.5E−02 |
| | | | 2, 165 kDa | | | | |
| 226034_at | 500 | | Homo sapiens, clone IMAGE: 3881549, mRNA | 376.1 | 212.0 | −1.77 | 1.5E−05 |
| 229778_at | 501 | MGC10946 | Hypothetical protein MGC10946 | 31.0 | 17.5 | −1.77 | 1.3E−03 |
| 204286_s_at | 502 | PMAIP1 | phorbol-12-myristate-13-acetate-induced protein 1 | 565.0 | 318.4 | −1.77 | 3.3E−05 |
| 217223_s_at | 503 | BCR | breakpoint cluster region | 151.1 | 85.1 | −1.78 | 1.9E−05 |
| 219878_s_at | 504 | KLF13 | Kruppel-like factor 13 | 239.9 | 135.1 | −1.78 | 2.2E−06 |
| 242352_at | 505 | NIPBL | Nipped-B homolog (Drosophila) | 130.5 | 73.3 | −1.78 | 3.0E−07 |
| 204860_s_at | 506 | BIRC1 | baculoviral IAP repeat-containing 1 | 158.1 | 88.7 | −1.78 | 6.0E−04 |
| 219228_at | 507 | ZNF331 | zinc finger protein 331 | 1917.2 | 1075.9 | −1.78 | 1.8E−05 |
| 210417_s_at | 508 | PIK4CB | phosphatidylinositol 4-kinase, catalytic, beta polypeptide | 105.9 | 59.3 | −1.78 | 1.2E−05 |
| 239555_at | 509 | LYN | V-yes-1 Yamaguchi sarcoma viral related oncogene | 229.5 | 128.5 | −1.79 | 7.0E−07 |
| | | | homolog | | | | |
| 208450_at | 510 | LGALS2 | lectin, galactoside-binding, soluble, 2 (galectin 2) | 202.6 | 113.4 | −1.79 | 8.7E−04 |
| 212000_at | 511 | SFRS14 | splicing factor, arginine/serine-rich 14 | 170.7 | 95.4 | −1.79 | 9.0E−07 |
| 218856_at | 512 | TNFRSF21 | tumor necrosis factor receptor superfamily, member 21 | 225.0 | 125.6 | −1.79 | 6.4E−06 |
| 218718_at | 513 | PDGFC | platelet derived growth factor C | 90.3 | 50.3 | −1.79 | 7.7E−05 |
| 205119_s_at | 514 | FPR1 | formyl peptide receptor 1 /// formyl peptide receptor 1 | 230.2 | 128.3 | −1.80 | 1.3E−04 |
| 1554309_at | 515 | EIF4G3 | eukaryotic translation initiation factor 4 gamma, 3 | 215.9 | 120.2 | −1.80 | 2.0E−07 |
| 223364_s_at | 516 | DHX37 | DEAH (Asp-Glu-Ala-His) box polypeptide 37 | 148.4 | 82.6 | −1.80 | 4.0E−07 |
| 207414_s_at | 517 | PCSK6 | proprotein convertase subtilisin/kexin type 6 | 51.8 | 28.8 | −1.80 | 4.6E−05 |
| 228195_at | 518 | MGC13057 | Hypothetical protein MGC13057 | 217.7 | 120.8 | −1.80 | 8.1E−04 |
| 204069_at | 519 | MEIS1 | Meis1, myeloid ecotropic viral integration site 1 homolog | 140.9 | 77.9 | −1.81 | 3.7E−04 |
| | | | (mouse) | | | | |
| 210338_s_at | 520 | HSPA8 | heat shock 70 kDa protein 8 | 462.4 | 255.6 | −1.81 | 1.5E−03 |
| 203066_at | 521 | GALNAC4S | B cell RAG associated protein | 597.0 | 330.1 | −1.81 | 2.3E−04 |
| 213300_at | 522 | KIAA0404 | KIAA0404 protein | 680.5 | 375.8 | −1.81 | 1.0E−06 |
| 226279_at | 523 | PRSS23 | protease, serine, 23 | 225.6 | 124.4 | −1.81 | 7.3E−06 |
| 206857_s_at | 524 | FKBP1B | FK506 binding protein 1B, 12.6 kDa | 130.8 | 72.1 | −1.82 | 3.2E−06 |
| 208776_at | 525 | PSMD11 | proteasome (prosome, macropain) 26S subunit, non-ATPase, | 1201.1 | 661.2 | −1.82 | <1.0E−07 |
| | | | 11 | | | | |
| 206722_s_at | 526 | EDG4 | endothelial differentiation, lysophosphatidic acid G-protein- | 261.0 | 143.6 | −1.82 | 9.0E−07 |
| | | | coupled receptor, 4 | | | | |
| 217427_s_at | 527 | HIRA | HIR histone cell cycle regulation defective homolog A | 289.4 | 159.0 | −1.82 | 1.4E−06 |
| | | | (S. cerevisiae) | | | | |
| 239027_at | 528 | DOCK8 | dedicator of cytokinesis 8 | 258.4 | 141.8 | −1.82 | 4.4E−05 |
| 217817_at | 529 | ARPC4 | actin related protein 2/3 complex, subunit 4, 20 kDa | 1429.8 | 784.1 | −1.82 | 4.0E−07 |
| 215023_s_at | 530 | PEX1 | peroxisome biogenesis factor 1 | 70.2 | 38.4 | −1.83 | 3.2E−05 |
| 230903_s_at | 531 | INM01 | Chromosome 8 open reading frame 42 | 127.9 | 70.0 | −1.83 | 3.6E−04 |
| 202455_at | 532 | HDAC5 | histone deacetylase 5 | 459.1 | 250.7 | −1.83 | 1.0E−07 |
| 204619_s_at | 533 | CSPG2 | chondroitin sulfate proteoglycan 2 (versican) | 114.3 | 62.4 | −1.83 | 3.6E−03 |
| 204663_at | 534 | ME3 | malic enzyme 3, NADP(+)-dependent, mitochondrial | 142.3 | 77.6 | −1.83 | 3.9E−06 |
| 205098_at | 535 | CCR1 | chemokine (C-C motif) receptor 1 | 108.4 | 59.1 | −1.84 | 1.1E−03 |
| 206366_x_at | 536 | XCL2 | chemokine (C motif) ligand 2 | 275.5 | 150.0 | −1.84 | 5.0E−07 |
| 204655_at | 537 | CCL5 | chemokine (C-C motif) ligand 5 /// chemokine (C-C motif) | 1263.2 | 687.0 | −1.84 | <1.0E−07 |
| | | | ligand 5 | | | | |
| 204875_s_at | 538 | GMDS | GDP-mannose 4,6-dehydratase | 203.2 | 110.4 | −1.84 | 3.8E−05 |
| 1405_i_at | 539 | CCL5 | chemokine (C-C motif) ligand 5 | 1231.0 | 668.9 | −1.84 | 1.3E−06 |
| 202897_at | 540 | PTPNS1 | protein tyrosine phosphatase, non-receptor type substrate 1 | 102.4 | 55.6 | −1.84 | 2.7E−04 |
| 203908_at | 541 | SLC4A4 | solute carrier family 4, sodium bicarbonate cotransporter, | 142.7 | 77.4 | −1.84 | 3.0E−07 |
| | | | member 4 | | | | |
| 210354_at | 542 | IFNG | interferon, gamma | 193.0 | 104.7 | −1.84 | 5.5E−03 |
| 212235_at | 543 | PLXND1 | plexin D1 | 486.8 | 264.1 | −1.84 | <1.0E−07 |
| 207428_x_at | 544 | CDC2L1 | cell division cycle 2-like 1 (PITSLRE proteins) | 237.8 | 128.9 | −1.84 | 3.0E−07 |
| 208714_at | 545 | NDUFV1 | NADH dehydrogenase (ubiquinone) flavoprotein 1, 51 kDa | 1750.6 | 948.9 | −1.84 | 2.0E−07 |
| 208960_s_at | 546 | KLF6 | Kruppel-like factor 6 | 96.8 | 52.4 | −1.85 | 2.3E−03 |
| 200885_at | 547 | RHOC | ras homolog gene family, member C | 272.8 | 147.6 | −1.85 | 1.0E−07 |
| 213891_s_at | 548 | TCF4 | Transcription factor 4 | 274.7 | 148.6 | −1.85 | <1.0E−07 |
| 211168_s_at | 549 | RENT1 | regulator of nonsense transcripts 1 | 1025.3 | 553.8 | −1.85 | 1.1E−05 |
| 209192_x_at | 550 | HTATIP | HIV-1 Tat interacting protein, 60 kDa | 148.6 | 80.2 | −1.85 | 3.0E−07 |
| 204993_at | 551 | GNAZ | guanine nucleotide binding protein (G protein), alpha z | 442.8 | 238.4 | −1.86 | 3.8E−05 |
| | | | polypeptide | | | | |
| 1552309_a_at | 552 | NEXN | nexilin (F actin binding protein) | 151.3 | 81.4 | −1.86 | 8.9E−05 |
| 212540_at | 553 | CDC34 | cell division cycle 34 | 348.0 | 186.9 | −1.86 | <1.0E−07 |
| 1555226_s_at | 554 | C1orf43 | chromosome 1 open reading frame 43 | 92.5 | 49.6 | −1.86 | 4.4E−04 |
| 216907_x_at | 555 | KIR3DL2 | killer cell immunoglobulin-like receptor, three domains, long | 150.2 | 80.4 | −1.87 | 1.2E−05 |
| | | | cytoplasmic tail, 2 | | | | |
| 227013_at | 556 | LATS2 | LATS, large tumor suppressor, homolog 2 (Drosophila) | 1475.0 | 789.5 | −1.87 | 1.0E−07 |
| 221214_s_at | 557 | NELF | nasal embryonic LHRH factor | 212.5 | 113.7 | −1.87 | 2.0E−07 |
| 201694_s_at | 558 | EGR1 | early growth response 1 | 1053.6 | 563.2 | −1.87 | 2.5E−04 |
| 201055_s_at | 559 | HNRPA0 | heterogeneous nuclear ribonucleoprotein A0 | 527.4 | 281.8 | −1.87 | 4.7E−06 |
| 218611_at | 560 | IER5 | immediate early response 5 | 2114.7 | 1128.6 | −1.87 | 3.0E−07 |
| 218272_at | 561 | FLJ20699 | hypothetical protein FLJ20699 | 937.5 | 498.5 | −1.88 | <1.0E−07 |
| 207857_at | 562 | LILRA2 | leukocyte immunoglobulin-like receptor, subfamily A (with | 112.7 | 59.9 | −1.88 | 4.8E−05 |
| | | | TM domain), member 2 | | | | |
| 37117_at | 563 | ARHGAP8 | Rho GTPase activating protein 8 /// PRR5-ARHGAP8 fusion | 101.6 | 53.9 | −1.88 | 1.0E−06 |
| 202084_s_at | 564 | SEC14L1 | SEC14-like 1 (S. cerevisiae) | 1229.8 | 652.6 | −1.88 | 2.1E−06 |
| 201583_s_at | 565 | SEC23B | Sec23 homolog B (S. cerevisiae) | 269.4 | 142.6 | −1.89 | 1.0E−07 |
| 222717_at | 566 | SDPR | serum deprivation response (phosphatidylserine binding | 185.6 | 98.1 | −1.89 | 1.4E−04 |
| | | | protein) | | | | |
| 1554821_a_at | 567 | ZBED1 | zinc finger, BED-type containing 1 | 136.5 | 72.1 | −1.89 | 6.0E−07 |
| 208478_s_at | 568 | BAX | BCL2-associated X protein | 77.2 | 40.7 | −1.89 | 8.3E−05 |
| 226068_at | 569 | SYK | Spleen tyrosine kinase | 449.2 | 237.1 | −1.89 | 6.0E−07 |
| 210627_s_at | 570 | GCS1 | glucosidase I | 328.4 | 172.4 | −1.91 | <1.0E−07 |
| 219256_s_at | 571 | SH3TC1 | SH3 domain and tetratricopeptide repeats 1 | 232.6 | 122.0 | −1.91 | 1.0E−07 |
| 201108_s_at | 572 | THBS1 | thrombospondin 1 | 205.3 | 107.7 | −1.91 | 1.4E−04 |
| 217854_s_at | 573 | POLR2E | polymerase (RNA) II (DNA directed) polypeptide E, 25 kDa | 547.0 | 286.0 | −1.91 | <1.0E−07 |
| 201059_at | 574 | CTTN | cortactin | 614.6 | 321.2 | −1.91 | 2.5E−05 |
| 237322_at | 575 | | Hypothetical protein LOC150271 | 171.4 | 89.6 | −1.91 | 8.5E−05 |
| 221969_at | 576 | PAX5 | Paired box gene 5 (B-cell lineage specific activator) | 196.3 | 102.5 | −1.91 | 1.0E−04 |
| 203680_at | 577 | PRKAR2B | protein kinase, cAMP-dependent, regulatory, type II, beta | 503.1 | 262.8 | −1.91 | 2.4E−04 |
| 1569599_at | 578 | SAMSN1 | SAM domain, SH3 domain and nuclear localisation signals, 1 | 30.6 | 16.0 | −1.92 | 3.0E−04 |
| 222407_s_at | 579 | ZFP106 | zinc finger protein 106 homolog (mouse) | 209.0 | 108.8 | −1.92 | 1.1E−05 |
| 211688_x_at | 580 | KIR3DL2 | | 187.9 | 97.8 | −1.92 | 4.2E−06 |
| 210624_s_at | 581 | ILVBL | ilvB (bacterial acetolactate synthase)-like | 512.6 | 266.7 | −1.92 | <1.0E−07 |
| 214487_s_at | 582 | RAP2A | RAP2A, member of RAS oncogene family | 106.5 | 55.3 | −1.93 | <1.0E−07 |
| 243107_at | 583 | CCR7 | Chemokine (C-C motif) receptor 7 | 65.2 | 33.8 | −1.93 | 5.6E−06 |
| 205349_at | 584 | GNA15 | guanine nucleotide binding protein (G protein), alpha 15 (Gq | 371.6 | 192.5 | −1.93 | <1.0E−07 |
| | | | class) | | | | |
| 1559910_at | 585 | FLJ20701 | Hypothetical protein FLJ20701 | 89.6 | 46.4 | −1.93 | 4.3E−04 |
| 211581_x_at | 586 | LST1 | leukocyte specific transcript 1 | 319.3 | 165.2 | −1.93 | 1.0E−07 |
| 224356_x_at | 587 | MS4A6A | membrane-spanning 4-domains, subfamily A, member 6A | 540.2 | 278.9 | −1.94 | 1.1E−04 |
| 212497_at | 588 | C14orf32 | chromosome 14 open reading frame 32 | 112.3 | 58.0 | −1.94 | 1.0E−07 |
| 213318_s_at | 589 | BAT3 | HLA-B associated transcript 3 | 1411.3 | 727.2 | −1.94 | <1.0E−07 |
| 201904_s_at | 590 | CTDSPL | carboxy-terminal domain, RNA polymerase II, polypeptide | 329.4 | 169.6 | −1.94 | 8.7E−05 |
| | | | A small phosphatase-like | | | | |
| 214146_s_at | 591 | PPBP | pro-platelet basic protein (chemokine (C—X—C motif) ligand | 2851.2 | 1468.0 | −1.94 | 9.6E−06 |
| | | | 7) | | | | |
| 216191_s_at | 592 | TCRA | T cell receptor alpha | 1196.0 | 615.3 | −1.94 | 7.7E−05 |
| 1559249_at | 593 | ATXN1 | Ataxin 1 | 286.0 | 147.0 | −1.95 | 1.5E−05 |
| 224823_at | 594 | MYLK | myosin, light polypeptide kinase | 413.2 | 211.9 | −1.95 | 1.7E−04 |
| 208627_s_at | 595 | YBX1 | Y box binding protein 1 | 748.0 | 383.0 | −1.95 | 6.7E−05 |
| 200661_at | 596 | PPGB | protective protein for beta-galactosidase (galactosialidosis) | 702.7 | 359.8 | −1.95 | 1.0E−07 |
| 1557910_at | 597 | HSPCB | heat shock 90 kDa protein 1, beta | 55.6 | 28.4 | −1.96 | 2.7E−04 |
| 208657_s_at | 598 | SEPT9 | septin 9 | 926.0 | 473.6 | −1.96 | <1.0E−07 |
| 202583_s_at | 599 | RANBP9 | RAN binding protein 9 | 87.7 | 44.8 | −1.96 | 1.7E−05 |
| 228170_at | 600 | OLIG1 | oligodendrocyte transcription factor 1 | 65.0 | 33.1 | −1.96 | 1.4E−03 |
| 201267_s_at | 601 | PSMC3 | proteasome (prosome, macropain) 26S subunit, ATPase, 3 | 129.7 | 66.1 | −1.96 | 4.1E−06 |
| 1554406_a_at | 602 | CLEC7A | C-type lectin domain family 7, member A | 176.2 | 89.7 | −1.96 | 3.8E−05 |
| 204115_at | 603 | GNG11 | guanine nucleotide binding protein (G protein), gamma 11 | 447.8 | 227.9 | −1.97 | 1.1E−04 |
| 214623_at | 604 | SHFM3P1 | split hand/foot malformation (ectrodactyly) type 3 | 197.2 | 100.3 | −1.97 | <1.0E−07 |
| | | | pseudogene 1 | | | | |
| 226188_at | 605 | HSPC159 | HSPC159 protein | 328.3 | 166.5 | −1.97 | 9.7E−05 |
| 225154_at | 606 | SYAP1 | synapse associated protein 1, SAP47 homolog (Drosophila) | 947.8 | 480.3 | −1.97 | 2.0E−07 |
| 223811_s_at | 607 | C7orf20 | chromosome 7 open reading frame 20 | 314.4 | 159.2 | −1.97 | 4.0E−07 |
| 221704_s_at | 608 | VPS37B | vacuolar protein sorting 37B (yeast) | 503.6 | 254.7 | −1.98 | <1.0E−07 |
| 212657_s_at | 609 | IL1RN | interleukin 1 receptor antagonist | 62.8 | 31.7 | −1.98 | 2.2E−04 |
| 214974_x_at | 610 | CXCL5 | chemokine (C—X—C motif) ligand 5 | 319.8 | 161.6 | −1.98 | 2.9E−05 |
| 206494_s_at | 611 | ITGA2B | integrin, alpha 2b | 58.7 | 29.6 | −1.98 | 1.8E−05 |
| 201417_at | 612 | SOX4 | SRY (sex determining region Y)-box 4 | 300.8 | 151.5 | −1.99 | 2.4E−05 |
| 205660_at | 613 | OASL | 2′-5′-oligoadenylate synthetase-like | 201.3 | 101.2 | −1.99 | 4.0E−04 |
| 202876_s_at | 614 | PBX2 | pre-B-cell leukemia transcription factor 2 | 385.1 | 193.4 | −1.99 | 3.0E−07 |
| 206445_s_at | 615 | HRMT1L2 | HMT1 hnRNP methyltransferase-like 2 (S. cerevisiae) | 694.8 | 348.8 | −1.99 | 5.0E−07 |
| 203561_at | 616 | FCGR2A | Fc fragment of IgG, low affinity IIa, receptor (CD32) | 129.8 | 65.1 | −1.99 | 3.3E−06 |
| 207460_at | 617 | GZMM | granzyme M (lymphocyte met-ase 1) | 287.9 | 143.8 | −2.00 | <1.0E−07 |
| 220494_s_at | 618 | | | 149.1 | 74.3 | −2.01 | 7.2E−03 |
| 202856_s_at | 619 | SLC16A3 | solute carrier family 16 (monocarboxylic acid transporters), | 135.3 | 67.4 | −2.01 | <1.0E−07 |
| | | | member 3 | | | | |
| 217356_s_at | 620 | PGK1 | phosphoglycerate kinase 1 | 135.0 | 67.3 | −2.01 | 1.2E−04 |
| 203045_at | 621 | NINJ1 | ninjurin 1 | 340.4 | 169.5 | −2.01 | <1.0E−07 |
| 213087_s_at | 622 | EEF1D | Eukaryotic translation elongation factor 1 delta | 63.1 | 31.4 | −2.01 | 8.1E−06 |
| 225354_s_at | 623 | SH3BGRL2 | SH3 domain binding glutamic acid-rich protein like 2 | 227.0 | 113.0 | −2.01 | 9.6E−05 |
| 227897_at | 624 | RAP2B | RAP2B, member of RAS oncogene family | 307.7 | 152.8 | −2.01 | 1.9E−05 |
| 238646_at | 625 | CLTC | Clathrin, heavy polypeptide (Hc) | 196.8 | 97.7 | −2.01 | 4.0E−07 |
| 202354_s_at | 626 | GTF2F1 | general transcription factor IIF, polypeptide 1, 74 kDa | 158.9 | 78.8 | −2.02 | 1.0E−07 |
| 237510_at | 627 | | | 336.9 | 167.0 | −2.02 | 2.0E−07 |
| 229441_at | 628 | PRSS23 | Protease, serine, 23 | 91.0 | 45.1 | −2.02 | <1.0E−07 |
| 204562_at | 629 | IRF4 | interferon regulatory factor 4 | 414.7 | 205.4 | −2.02 | 6.0E−07 |
| 242705_x_at | 630 | LRPAP1 | Low density lipoprotein receptor-related protein associated | 167.9 | 83.1 | −2.02 | <1.0E−07 |
| | | | protein 1 | | | | |
| 1553589_a_at | 631 | PDZK1IP1 | PDZK1 interacting protein 1 | 116.3 | 57.6 | −2.02 | 8.3E−03 |
| 212509_s_at | 632 | MXRA7 | matrix-remodelling associated 7 | 604.0 | 298.9 | −2.02 | <1.0E−07 |
| 223553_s_at | 633 | DOK3 | docking protein 3 | 211.4 | 104.6 | −2.02 | 4.3E−05 |
| 229733_s_at | 634 | CBX6 | Chromobox homolog 6 | 78.8 | 39.0 | −2.02 | 2.6E−03 |
| 220712_at | 635 | C8orf60 | chromosome 8 open reading frame 60 | 327.3 | 161.4 | −2.03 | <1.0E−07 |
| 212761_at | 636 | TCF7L2 | transcription factor 7-like 2 (T-cell specific, HMG-box) | 2472.6 | 1218.6 | −2.03 | <1.0E−07 |
| 238669_at | 637 | PTGS1 | prostaglandin-endoperoxide synthase 1 | 712.9 | 351.2 | −2.03 | 8.3E−06 |
| 213175_s_at | 638 | SNRPB | small nuclear ribonucleoprotein polypeptides B and B1 | 3194.1 | 1572.8 | −2.03 | 4.5E−06 |
| 205220_at | 639 | GPR109B | G protein-coupled receptor 109B | 75.0 | 36.9 | −2.03 | 2.1E−05 |
| 203139_at | 640 | DAPK1 | death-associated protein kinase 1 | 357.5 | 175.9 | −2.03 | 1.8E−06 |
| 203574_at | 641 | NFIL3 | nuclear factor, interleukin 3 regulated | 378.1 | 185.8 | −2.04 | 1.0E−07 |
| 219099_at | 642 | C12orf5 | chromosome 12 open reading frame 5 | 469.5 | 230.6 | −2.04 | <1.0E−07 |
| 204760_s_at | 643 | THRA | thyroid hormone receptor, alpha | 417.0 | 204.7 | −2.04 | <1.0E−07 |
| 224563_at | 644 | WASF2 | WAS protein family, member 2 | 95.2 | 46.7 | −2.04 | 2.0E−07 |
| 1562255_at | 645 | SYTL3 | synaptotagmin-like 3 | 184.4 | 90.3 | −2.04 | 1.1E−05 |
| 210785_s_at | 646 | C1orf38 | chromosome 1 open reading frame 38 | 398.8 | 195.2 | −2.04 | 8.0E−07 |
| 1554260_a_at | 647 | KIAA0826 | KIAA0826 | 83.5 | 40.8 | −2.05 | 1.0E−07 |
| 244470_at | 648 | RNF12 | Ring finger protein 12 | 1139.6 | 555.5 | −2.05 | 2.0E−07 |
| 1558719_s_at | 649 | C1QBP | Complement component 1, q subcomponent binding protein | 173.3 | 84.4 | −2.05 | 2.5E−06 |
| 228056_s_at | 650 | NAPSB | napsin B aspartic peptidase pseudogene | 343.4 | 167.2 | −2.05 | 2.0E−07 |
| 207314_x_at | 651 | KIR3DL2 | killer cell immunoglobulin-like receptor, three domains, long | 272.3 | 132.5 | −2.05 | 1.4E−06 |
| | | | cytoplasmic tail, 2 | | | | |
| 213418_at | 652 | HSPA6 | heat shock 70 kDa protein 6 (HSP70B′) | 345.8 | 168.1 | −2.06 | 5.3E−06 |
| 218895_at | 653 | GPATC3 | G patch domain containing 3 | 192.3 | 93.3 | −2.06 | <1.0E−07 |
| 211582_x_at | 654 | LST1 | leukocyte specific transcript 1 | 468.9 | 227.3 | −2.06 | 2.0E−07 |
| 202910_s_at | 655 | CD97 | CD97 antigen | 1457.3 | 705.2 | −2.07 | <1.0E−07 |
| 242832_at | 656 | PER1 | period homolog 1 (Drosophila) | 358.9 | 173.6 | −2.07 | <1.0E−07 |
| 223454_at | 657 | CXCL16 | chemokine (C—X—C motif) ligand 16 | 221.1 | 106.9 | −2.07 | 1.0E−07 |
| 221211_s_at | 658 | C21orf7 | chromosome 21 open reading frame 7 | 78.8 | 38.0 | −2.07 | 2.9E−04 |
| 202993_at | 659 | ILVBL | ilvB (bacterial acetolactate synthase)-like | 360.2 | 173.8 | −2.07 | 2.0E−07 |
| 200736_s_at | 660 | GPX1 | glutathione peroxidase 1 | 4595.0 | 2211.6 | −2.08 | 1.5E−05 |
| 225063_at | 661 | UBL7 | ubiquitin-like 7 (bone marrow stromal cell-derived) | 204.1 | 98.0 | −2.08 | <1.0E−07 |
| 217831_s_at | 662 | NSFL1C | NSFL1 (p97) cofactor (p47) | 125.3 | 60.2 | −2.08 | 1.0E−07 |
| 36829_at | 663 | PER1 | period homolog 1 (Drosophila) | 1374.7 | 659.2 | −2.09 | 3.0E−07 |
| 209651_at | 664 | TGFB1I1 | transforming growth factor beta 1 induced transcript 1 | 42.7 | 20.4 | −2.09 | <1.0E−07 |
| 217853_at | 665 | TENS1 | Tensin 3 | 243.3 | 116.5 | −2.09 | 4.5E−06 |
| 244546_at | 666 | CYCS | cytochrome c, somatic | 312.0 | 149.3 | −2.09 | 6.0E−07 |
| 222113_s_at | 667 | EPS15L1 | epidermal growth factor receptor pathway substrate 15-like 1 | 173.9 | 83.1 | −2.09 | <1.0E−07 |
| 220091_at | 668 | SLC2A6 | solute carrier family 2 (facilitated glucose transporter), | 256.1 | 122.3 | −2.09 | 1.0E−07 |
| | | | member 6 | | | | |
| 207111_at | 669 | EMR1 | egf-like module containing, mucin-like, hormone receptor- | 329.2 | 157.2 | −2.09 | 1.0E−07 |
| | | | like 1 | | | | |
| 227180_at | 670 | ELOVL7 | ELOVL family member 7, elongation of long chain fatty | 299.3 | 142.9 | −2.09 | 2.2E−05 |
| | | | acids (yeast) | | | | |
| 205821_at | 671 | KLRK1 | killer cell lectin-like receptor subfamily K, member 1 | 1120.3 | 534.5 | −2.10 | <1.0E−07 |
| 206390_x_at | 672 | PF4 | platelet factor 4 (chemokine (C—X—C motif) ligand 4) | 2309.3 | 1097.7 | −2.10 | 2.5E−06 |
| 201401_s_at | 673 | ADRBK1 | adrenergic, beta, receptor kinase 1 | 217.4 | 103.2 | −2.11 | <1.0E−07 |
| 212384_at | 674 | BAT1 | HLA-B associated transcript 1 | 824.0 | 390.6 | −2.11 | 2.0E−07 |
| 212041_at | 675 | ATP6V0D1 | ATPase, H+ transporting, lysosomal 38 kDa, V0 subunit d | 1045.3 | 495.5 | −2.11 | 5.4E−06 |
| | | | isoform 1 | | | | |
| 201137_s_at | 676 | HLA-DPB1 | major histocompatibility complex, class II, DP beta 1 | 1414.5 | 669.7 | −2.11 | 1.0E−07 |
| 208885_at | 677 | LCP1 | lymphocyte cytosolic protein 1 (L-plastin) | 423.0 | 200.3 | −2.11 | 1.2E−05 |
| 223368_s_at | 678 | AD-003 | AD-003 protein | 480.8 | 227.4 | −2.11 | <1.0E−07 |
| 239451_at | 679 | TRA1 | Tumor rejection antigen (gp96) 1 | 188.9 | 89.3 | −2.12 | 1.1E−05 |
| 202912_at | 680 | ADM | adrenomedullin | 193.5 | 91.5 | −2.12 | 2.3E−05 |
| 230833_at | 681 | ACRBP | acrosin binding protein | 144.4 | 68.2 | −2.12 | 9.2E−06 |
| 209774_x_at | 682 | CXCL2 | chemokine (C—X—C motif) ligand 2 | 36.0 | 17.0 | −2.12 | 1.1E−04 |
| 224703_at | 683 | WDR22 | WD repeat domain 22 | 393.2 | 185.5 | −2.12 | <1.0E−07 |
| 200878_at | 684 | EPAS1 | endothelial PAS domain protein 1 | 73.0 | 34.4 | −2.12 | <1.0E−07 |
| 227404_s_at | 685 | EGR1 | Early growth response 1 | 168.9 | 79.4 | −2.13 | 2.3E−04 |
| 1568870_at | 686 | | CDNA clone IMAGE: 5260228 | 93.9 | 44.1 | −2.13 | 2.0E−05 |
| 214574_x_at | 687 | LST1 | leukocyte specific transcript 1 | 562.1 | 264.0 | −2.13 | 1.0E−07 |
| 204852_s_at | 688 | PTPN7 | protein tyrosine phosphatase, non-receptor type 7 | 258.9 | 121.4 | −2.13 | <1.0E−07 |
| 204541_at | 689 | SEC14L2 | SEC14-like 2 (S. cerevisiae) | 107.3 | 50.3 | −2.13 | <1.0E−07 |
| 1569030_s_at | 690 | NYREN18 | NEDD8 ultimate buster-1 | 936.9 | 438.5 | −2.14 | <1.0E−07 |
| 212827_at | 691 | IGHM | immunoglobulin heavy constant mu | 523.7 | 245.1 | −2.14 | 2.2E−06 |
| 221712_s_at | 692 | WDR74 | WD repeat domain 74 /// WD repeat domain 74 | 531.5 | 248.6 | −2.14 | <1.0E−07 |
| 208579_x_at | 693 | H2BFS | H2B histone family, member S | 116.2 | 54.3 | −2.14 | <1.0E−07 |
| 204446_s_at | 694 | ALOX5 | arachidonate 5-lipoxygenase | 772.5 | 360.8 | −2.14 | <1.0E−07 |
| 221765_at | 695 | UGCG | UDP-glucose ceramide glucosyltransferase | 97.8 | 45.6 | −2.14 | 2.0E−07 |
| 208722_s_at | 696 | ANAPC5 | anaphase promoting complex subunit 5 | 760.5 | 353.9 | −2.15 | <1.0E−07 |
| 212975_at | 697 | DENND3 | DENN/MADD domain containing 3 | 541.1 | 251.8 | −2.15 | <1.0E−07 |
| 1557261_at | 698 | LOC339005 | hypothetical protein LOC339005 | 191.2 | 88.8 | −2.15 | 3.3E−05 |
| 38671_at | 699 | PLXND1 | plexin D1 | 765.1 | 355.2 | −2.15 | <1.0E−07 |
| 206881_s_at | 700 | LILRA3 | leukocyte immunoglobulin-like receptor, subfamily A, | 133.3 | 61.8 | −2.16 | 3.0E−07 |
| | | | member 3 | | | | |
| 223048_at | 701 | FLJ20487 | hypothetical protein FLJ20487 | 437.9 | 202.7 | −2.16 | <1.0E−07 |
| 233749_at | 702 | LOC442456 | similar to bA368D24A.1 (novel protein) | 93.8 | 43.2 | −2.17 | 1.0E−06 |
| 201245_s_at | 703 | OTUB1 | OTU domain, ubiquitin aldehyde binding 1 | 480.2 | 221.2 | −2.17 | 1.0E−07 |
| 228746_s_at | 704 | H41 | Hypothetical protein H41 | 84.6 | 39.0 | −2.17 | 3.6E−04 |
| 233177_s_at | 705 | MR-1 | myofibrillogenesis regulator 1 | 200.5 | 92.3 | −2.17 | 1.0E−07 |
| 230511_at | 706 | CREM | cAMP responsive element modulator | 1560.8 | 718.5 | −2.17 | 1.7E−06 |
| 1569346_a_at | 707 | P2RX1 | Purinergic receptor P2X, ligand-gated ion channel, 1 | 67.9 | 31.2 | −2.18 | <1.0E−07 |
| 222916_s_at | 708 | HDLBP | high density lipoprotein binding protein (vigilin) | 64.2 | 29.5 | −2.18 | <1.0E−07 |
| 209257_s_at | 709 | CSPG6 | chondroitin sulfate proteoglycan 6 (bamacan) | 116.4 | 53.5 | −2.18 | 8.0E−07 |
| 232627_at | 710 | HGS | Hepatocyte growth factor-regulated tyrosine kinase substrate | 246.2 | 113.1 | −2.18 | <1.0E−07 |
| 208792_s_at | 711 | CLU | clusterin | 390.9 | 179.5 | −2.18 | 5.8E−05 |
| 209020_at | 712 | C20orf111 | chromosome 20 open reading frame 111 | 1142.2 | 524.1 | −2.18 | <1.0E−07 |
| 218020_s_at | 713 | TEX27 | testis expressed sequence 27 | 230.5 | 105.7 | −2.18 | <1.0E−07 |
| 1555349_a_at | 714 | ITGB2 | integrin, beta 2 (antigen CD18) | 448.7 | 205.6 | −2.18 | 3.6E−06 |
| 226053_at | 715 | MAP2K7 | mitogen-activated protein kinase kinase 7 | 250.6 | 114.8 | −2.18 | <1.0E−07 |
| 1554423_a_at | 716 | FBXO7 | F-box protein 7 | 756.1 | 345.7 | −2.19 | <1.0E−07 |
| 202626_s_at | 717 | LYN | v-yes-1 Yamaguchi sarcoma viral related oncogene homolog | 1907.3 | 870.5 | −2.19 | <1.0E−07 |
| 204535_s_at | 718 | REST | RE1-silencing transcription factor | 212.9 | 97.0 | −2.19 | <1.0E−07 |
| 202301_s_at | 719 | FLJ11021 | similar to splicing factor, arginine/serine-rich 4 | 824.3 | 375.7 | −2.19 | <1.0E−07 |
| 211748_x_at | 720 | PTGDS | prostaglandin D2 synthase 21 kDa (brain) | 794.4 | 361.7 | −2.20 | <1.0E−07 |
| 202284_s_at | 721 | CDKN1A | cyclin-dependent kinase inhibitor 1A (p21, Cip1) | 513.0 | 233.6 | −2.20 | <1.0E−07 |
| 214181_x_at | 722 | LST1 | leukocyte specific transcript 1 | 503.1 | 229.0 | −2.20 | <1.0E−07 |
| 223369_at | 723 | AD-003 | AD-003 protein | 281.7 | 127.9 | −2.20 | <1.0E−07 |
| 200658_s_at | 724 | PHB | prohibitin | 404.2 | 183.4 | −2.20 | 3.0E−07 |
| 212695_at | 725 | CRY2 | cryptochrome 2 (photolyase-like) | 344.3 | 156.0 | −2.21 | <1.0E−07 |
| 203585_at | 726 | ZNF185 | zinc finger protein 185 (LIM domain) | 332.0 | 150.2 | −2.21 | 7.0E−07 |
| 230245_s_at | 727 | LOC283663 | hypothetical protein LOC283663 | 883.5 | 399.0 | −2.21 | 4.0E−07 |
| 201416_at | 728 | SOX4 | SRY (sex determining region Y)-box 4 | 1730.9 | 780.2 | −2.22 | <1.0E−07 |
| 201751_at | 729 | KIAA0063 | KIAA0063 gene product | 1852.9 | 833.3 | −2.22 | <1.0E−07 |
| 220532_s_at | 730 | LR8 | LR8 protein | 116.8 | 52.5 | −2.22 | 4.2E−03 |
| 209959_at | 731 | NR4A3 | nuclear receptor subfamily 4, group A, member 3 | 162.7 | 72.9 | −2.23 | 1.0E−07 |
| 213338_at | 732 | RIS1 | Ras-induced senescence 1 | 272.8 | 122.2 | −2.23 | 2.6E−05 |
| 36711_at | 733 | MAFF | v-maf musculoaponeurotic fibrosarcoma oncogene homolog F | 5920.0 | 2650.8 | −2.23 | <1.0E−07 |
| 220953_s_at | 734 | MTMR12 | myotubularin related protein 12 | 166.2 | 74.4 | −2.23 | <1.0E−07 |
| 212085_at | 735 | SLC25A6 | solute carrier family 25, member 6 | 4293.9 | 1921.3 | −2.23 | 1.2E−06 |
| 204103_at | 736 | CCL4 | chemokine (C-C motif) ligand 4 | 1023.7 | 457.6 | −2.24 | 1.0E−07 |
| 208690_s_at | 737 | PDLIM1 | PDZ and LIM domain 1 (elfin) | 1160.3 | 516.5 | −2.25 | <1.0E−07 |
| 206465_at | 738 | ACSBG1 | acyl-CoA synthetase bubblegum family member 1 | 64.7 | 28.7 | −2.25 | 9.0E−06 |
| 230690_at | 739 | TUBB1 | tubulin, beta 1 | 1947.5 | 865.4 | −2.25 | 1.5E−05 |
| 211654_x_at | 740 | HLA-DQB1 | major histocompatibility complex, class II, DQ beta 1 | 397.5 | 176.4 | −2.25 | 3.9E−06 |
| 226858_at | 741 | CSNK1E | Casein kinase 1, epsilon | 601.6 | 266.8 | −2.26 | <1.0E−07 |
| 207697_x_at | 742 | LILRB2 | leukocyte immunoglobulin-like receptor, subfamily B, | 137.1 | 60.6 | −2.26 | <1.0E−07 |
| | | | member 2 | | | | |
| 212025_s_at | 743 | FLII | flightless I homolog (Drosophila) | 98.3 | 43.4 | −2.26 | 1.0E−07 |
| 206486_at | 744 | LAG3 | lymphocyte-activation gene 3 | 110.9 | 48.9 | −2.26 | <1.0E−07 |
| 202068_s_at | 745 | LDLR | low density lipoprotein receptor (familial | 200.4 | 88.4 | −2.27 | <1.0E−07 |
| | | | hypercholesterolemia) | | | | |
| 201755_at | 746 | MCM5 | MCM5 minichromosome maintenance deficient 5, cell | 234.2 | 103.1 | −2.27 | <1.0E−07 |
| | | | division cycle 46 | | | | |
| 217591_at | 747 | SKIL | SKI-like | 1484.9 | 652.7 | −2.27 | <1.0E−07 |
| 209383_at | 748 | DDIT3 | DNA-damage-inducible transcript 3 | 339.9 | 149.3 | −2.28 | <1.0E−07 |
| 226011_at | 749 | CCDC12 | coiled-coil domain containing 12 | 1372.4 | 602.2 | −2.28 | <1.0E−07 |
| 205193_at | 750 | MAFF | v-maf musculoaponeurotic fibrosarcoma oncogene homolog F | 486.0 | 213.2 | −2.28 | <1.0E−07 |
| 213348_at | 751 | CDKN1C | Cyclin-dependent kinase inhibitor 1C (p57, Kip2) | 271.3 | 119.0 | −2.28 | 2.0E−07 |
| 211833_s_at | 752 | BAX | BCL2-associated X protein | 349.6 | 153.4 | −2.28 | 2.0E−07 |
| 205883_at | 753 | ZBTB16 | zinc finger and BTB domain containing 16 | 525.1 | 229.2 | −2.29 | <1.0E−07 |
| 202708_s_at | 754 | HIST2H2BE | histone 2, H2be | 746.8 | 325.6 | −2.29 | 6.6E−06 |
| 221432_s_at | 755 | SLC25A28 | solute carrier family 25, member 28 | 430.7 | 187.6 | −2.30 | 2.0E−07 |
| 215210_s_at | 756 | DLST | dihydrolipoamide S-succinyltransferase | 544.5 | 236.9 | −2.30 | <1.0E−07 |
| 208869_s_at | 757 | GABARAPL1 | GABA(A) receptor-associated protein like 1 | 514.8 | 224.0 | −2.30 | <1.0E−07 |
| 212002_at | 758 | C1orf144 | chromosome 1 open reading frame 144 | 759.2 | 330.3 | −2.30 | 1.8E−06 |
| 210046_s_at | 759 | IDH2 | isocitrate dehydrogenase 2 (NADP+), mitochondrial | 555.4 | 241.6 | −2.30 | <1.0E−07 |
| 212187_x_at | 760 | PTGDS | prostaglandin D2 synthase 21 kDa (brain) | 353.7 | 153.8 | −2.30 | <1.0E−07 |
| 224980_at | 761 | LEMD2 | LEM domain containing 2 | 120.6 | 52.4 | −2.30 | 5.1E−06 |
| 241133_at | 762 | | CDNA FLJ35984 fis, clone TESTI2014097 | 290.9 | 126.3 | −2.30 | 3.5E−06 |
| 1568768_s_at | 763 | BRE | brain and reproductive organ-expressed (TNFRSF1A | 336.0 | 145.9 | −2.30 | 3.0E−07 |
| | | | modulator) | | | | |
| 220248_x_at | 764 | NSFL1C | NSFL1 (p97) cofactor (p47) | 361.7 | 157.0 | −2.30 | <1.0E−07 |
| 1552807_a_at | 765 | SIGLEC10 | sialic acid binding Ig-like lectin 10 | 453.1 | 196.5 | −2.31 | <1.0E−07 |
| 230972_at | 766 | ANKRD9 | ankyrin repeat domain 9 | 209.4 | 90.7 | −2.31 | <1.0E−07 |
| 219529_at | 767 | CLIC3 | chloride intracellular channel 3 | 112.6 | 48.8 | −2.31 | 7.0E−07 |
| 201095_at | 768 | DAP | death-associated protein | 420.0 | 181.6 | −2.31 | <1.0E−07 |
| 202283_at | 769 | SERPINF1 | serpin peptidase inhibitor, clade F member 1 | 113.2 | 48.8 | −2.32 | <1.0E−07 |
| 228376_at | 770 | GGTA1 | Glycoprotein, alpha-galactosyltransferase 1 | 247.1 | 106.5 | −2.32 | 6.3E−06 |
| 238893_at | 771 | LOC338758 | hypothetical protein LOC338758 | 1714.0 | 737.6 | −2.32 | <1.0E−07 |
| 232311_at | 772 | B2M | Beta-2-microglobulin | 499.7 | 215.0 | −2.32 | <1.0E−07 |
| 220016_at | 773 | AHNAK | AHNAK nucleoprotein (desmoyokin) | 325.9 | 140.2 | −2.32 | <1.0E−07 |
| 243857_at | 774 | MORF4L2 | Mortality factor 4 like 2 | 86.4 | 37.1 | −2.33 | 7.0E−07 |
| 217362_x_at | 775 | HLA-DRB6 | major histocompatibility complex, class II, DR beta 6 | 456.3 | 195.8 | −2.33 | <1.0E−07 |
| | | | (pseudogene) | | | | |
| 227101_at | 776 | LOC168850 | hypothetical protein LOC168850 | 80.0 | 34.4 | −2.33 | <1.0E−07 |
| 204627_s_at | 777 | ITGB3 | integrin, beta 3 (platelet glycoprotein IIIa, antigen CD61) | 169.9 | 72.9 | −2.33 | 3.9E−06 |
| 204383_at | 778 | DGCR14 | DiGeorge syndrome critical region gene 14 | 149.3 | 64.0 | −2.33 | <1.0E−07 |
| 201204_s_at | 779 | RRBP1 | Ribosome binding protein 1 homolog 180 kDa (dog) | 197.8 | 84.8 | −2.33 | <1.0E−07 |
| 227799_at | 780 | MYO1G | myosin IG | 368.1 | 157.6 | −2.34 | 1.5E−05 |
| 204440_at | 781 | CD83 | CD83 antigen | 879.9 | 376.5 | −2.34 | <1.0E−07 |
| 213915_at | 782 | NKG7 | natural killer cell group 7 sequence | 3867.3 | 1653.3 | −2.34 | 1.0E−07 |
| 202982_s_at | 783 | ACOT2 | acyl-CoA thioesterase 2 | 392.7 | 167.3 | −2.35 | <1.0E−07 |
| 225954_s_at | 784 | MIDN | midnolin | 363.4 | 154.6 | −2.35 | <1.0E−07 |
| 222874_s_at | 785 | CLN8 | ceroid-lipofuscinosis, neuronal 8 | 115.8 | 49.3 | −2.35 | <1.0E−07 |
| 205936_s_at | 786 | HK3 | hexokinase 3 (white cell) | 508.7 | 216.3 | −2.35 | 1.6E−06 |
| 217763_s_at | 787 | RAB31 | RAB31, member RAS oncogene family | 326.1 | 138.6 | −2.35 | 1.6E−05 |
| 226389_s_at | 788 | RAPGEF1 | Rap guanine nucleotide exchange factor (GEF) 1 | 694.6 | 294.1 | −2.36 | <1.0E−07 |
| 218009_s_at | 789 | PRC1 | protein regulator of cytokinesis 1 | 324.6 | 137.1 | −2.37 | <1.0E−07 |
| 212581_x_at | 790 | GAPDH | glyceraldehyde-3-phosphate dehydrogenase | 7043.2 | 2969.8 | −2.37 | <1.0E−07 |
| 201028_s_at | 791 | CD99 | CD99 antigen | 651.2 | 274.5 | −2.37 | 4.0E−07 |
| 225173_at | 792 | ARHGAP18 | Rho GTPase activating protein 18 | 180.9 | 76.3 | −2.37 | <1.0E−07 |
| 227364_at | 793 | | | 276.5 | 116.4 | −2.38 | 4.0E−07 |
| 201118_at | 794 | PGD | phosphogluconate dehydrogenase | 160.0 | 67.2 | −2.38 | <1.0E−07 |
| 231727_s_at | 795 | AD023 | AD023 protein | 265.9 | 111.7 | −2.38 | <1.0E−07 |
| 207979_s_at | 796 | CD8B1 | CD8 antigen, beta polypeptide 1 (p37) | 796.9 | 334.6 | −2.38 | 8.0E−07 |
| 207815_at | 797 | PF4V1 | platelet factor 4 variant 1 | 194.0 | 81.4 | −2.38 | 7.1E−04 |
| 1555613_a_at | 798 | ZAP70 | zeta-chain (TCR) associated protein kinase 70 kDa | 883.4 | 370.5 | −2.38 | 1.7E−05 |
| 204838_s_at | 799 | MLH3 | mutL homolog 3 (E. coli) | 571.4 | 239.6 | −2.38 | 5.0E−07 |
| 32032_at | 800 | DGCR14 | DiGeorge syndrome critical region gene 14 | 505.9 | 212.1 | −2.39 | <1.0E−07 |
| 210321_at | 801 | GZMH | granzyme H (cathepsin G-like 2, protein h-CCPX) | 1997.6 | 836.8 | −2.39 | <1.0E−07 |
| 209949_at | 802 | NCF2 | neutrophil cytosolic factor 2 | 497.3 | 208.2 | −2.39 | 4.0E−07 |
| 208622_s_at | 803 | VIL2 | villin 2 (ezrin) | 1065.9 | 445.7 | −2.39 | <1.0E−07 |
| 235014_at | 804 | LOC147727 | hypothetical protein LOC147727 | 267.2 | 111.4 | −2.40 | <1.0E−07 |
| 211656_x_at | 805 | HLA-DQB1 | major histocompatibility complex, class II, DQ beta 1 | 410.8 | 169.8 | −2.42 | <1.0E−07 |
| 218280_x_at | 806 | HIST2H2AA | histone 2, H2aa | 689.1 | 284.5 | −2.42 | <1.0E−07 |
| 218454_at | 807 | FLJ22662 | hypothetical protein FLJ22662 | 739.1 | 305.2 | −2.42 | 1.4E−05 |
| 215092_s_at | 808 | NFAT5 | nuclear factor of activated T-cells 5 | 120.6 | 49.8 | −2.42 | <1.0E−07 |
| 222043_at | 809 | CLU | clusterin | 57.9 | 23.9 | −2.42 | 5.7E−06 |
| 204039_at | 810 | CEBPA | CCAAT/enhancer binding protein (C/EBP), alpha | 207.8 | 85.7 | −2.42 | <1.0E−07 |
| 230574_at | 811 | LOC388480 | hypothetical LOC388480 | 62.9 | 25.9 | −2.43 | 3.6E−06 |
| 225738_at | 812 | RAPGEF1 | Rap guanine nucleotide exchange factor (GEF) 1 | 1133.9 | 467.3 | −2.43 | <1.0E−07 |
| 211192_s_at | 813 | CD84 | CD84 antigen (leukocyte antigen) | 51.4 | 21.1 | −2.43 | <1.0E−07 |
| 209953_s_at | 814 | CDC37 | CDC37 cell division cycle 37 homolog (S. cerevisiae) | 191.5 | 78.8 | −2.43 | 1.4E−06 |
| 201465_s_at | 815 | JUN | v-jun sarcoma virus 17 oncogene homolog (avian) | 302.6 | 124.3 | −2.43 | 1.1E−06 |
| 201264_at | 816 | COPE | coatomer protein complex, subunit epsilon | 391.5 | 160.5 | −2.44 | <1.0E−07 |
| 200866_s_at | 817 | PSAP | prosaposin | 381.7 | 156.2 | −2.44 | <1.0E−07 |
| 207206_s_at | 818 | ALOX12 | arachidonate 12-lipoxygenase | 250.5 | 102.3 | −2.45 | 4.0E−06 |
| 217716_s_at | 819 | SEC61A1 | Sec61 alpha 1 subunit (S. cerevisiae) | 338.6 | 138.0 | −2.45 | 6.0E−07 |
| 218064_s_at | 820 | AKAP8L | A kinase (PRKA) anchor protein 8-like | 137.1 | 55.8 | −2.46 | 4.8E−06 |
| 202510_s_at | 821 | TNFAIP2 | tumor necrosis factor, alpha-induced protein 2 | 285.5 | 116.0 | −2.46 | 2.0E−07 |
| 200982_s_at | 822 | ANXA6 | annexin A6 | 404.7 | 163.7 | −2.47 | 1.0E−07 |
| 1555759_a_at | 823 | CCL5 | chemokine (C-C motif) ligand 5 | 2643.5 | 1067.3 | −2.48 | 3.0E−07 |
| 1555962_at | 824 | B3GNT7 | UDP-GlcNAc:betaGal beta-1,3-N- | 143.8 | 58.0 | −2.48 | <1.0E−07 |
| | | | acetylglucosaminyltransferase 7 | | | | |
| 208161_s_at | 825 | ABCC3 | ATP-binding cassette, sub-family C (CFTR/MRP), member 3 | 205.8 | 83.0 | −2.48 | <1.0E−07 |
| 206110_at | 826 | HIST1H3H | histone 1, H3h | 659.4 | 265.8 | −2.48 | 1.1E−05 |
| 208624_s_at | 827 | EIF4G1 | eukaryotic translation initiation factor 4 gamma, 1 | 242.3 | 97.4 | −2.49 | <1.0E−07 |
| 204890_s_at | 828 | LCK | lymphocyte-specific protein tyrosine kinase | 727.7 | 292.4 | −2.49 | 4.5E−06 |
| 234942_s_at | 829 | DNTTIP1 | deoxynucleotidyltransferase, terminal, interacting protein 1 | 122.9 | 49.2 | −2.50 | <1.0E−07 |
| 204588_s_at | 830 | SLC7A7 | solute carrier family 7 (cationic amino acid transporter, y+ | 951.0 | 380.4 | −2.50 | 9.0E−07 |
| | | | system), member 7 | | | | |
| 221748_s_at | 831 | TNS1 | tensin 1 /// tensin 1 | 109.7 | 43.9 | −2.50 | 1.0E−06 |
| 201340_s_at | 832 | ENC1 | ectodermal-neural cortex (with BTB-like domain) | 177.2 | 70.8 | −2.50 | 1.0E−07 |
| 204638_at | 833 | ACP5 | acid phosphatase 5, tartrate resistant | 369.5 | 147.3 | −2.51 | <1.0E−07 |
| 204971_at | 834 | CSTA | cystatin A (stefin A) | 751.1 | 298.9 | −2.51 | 3.2E−06 |
| 201195_s_at | 835 | SLC7A5 | solute carrier family 7 member 5 | 1028.5 | 409.0 | −2.51 | <1.0E−07 |
| 200665_s_at | 836 | SPARC | osteonectin | 1365.2 | 542.0 | −2.52 | 5.0E−07 |
| 218028_at | 837 | ELOVL1 | elongation of very long chain fatty acids (FEN1/Elo2, | 291.2 | 115.5 | −2.52 | <1.0E−07 |
| | | | SUR4/Elo3, yeast)-like 1 | | | | |
| 210895_s_at | 838 | CD86 | CD86 antigen (CD28 antigen ligand 2, B7-2 antigen) | 94.0 | 37.2 | −2.53 | 1.3E−06 |
| 213453_x_at | 839 | GAPDH | glyceraldehyde-3-phosphate dehydrogenase | 4683.0 | 1853.4 | −2.53 | <1.0E−07 |
| 212065_s_at | 840 | USP34 | ubiquitin specific peptidase 34 | 95.7 | 37.8 | −2.53 | <1.0E−07 |
| 203535_at | 841 | S100A9 | S100 calcium binding protein A9 (calgranulin B) | 603.6 | 238.3 | −2.53 | 2.2E−06 |
| 222670_s_at | 842 | MAFB | v-maf musculoaponeurotic fibrosarcoma oncogene homolog | 566.5 | 223.6 | −2.53 | 6.0E−07 |
| | | | B (avian) | | | | |
| 217078_s_at | 843 | CD300A | CD300A antigen | 59.3 | 23.4 | −2.54 | 1.0E−06 |
| 228284_at | 844 | TLE1 | transducin-like enhancer of split 1 | 246.7 | 97.1 | −2.54 | <1.0E−07 |
| 209160_at | 845 | AKR1C3 | aldo-keto reductase family 1, member C3 | 468.3 | 183.8 | −2.55 | <1.0E−07 |
| 205863_at | 846 | S100A12 | S100 calcium binding protein A12 (calgranulin C) | 188.5 | 74.0 | −2.55 | 7.1E−06 |
| 222218_s_at | 847 | PILRA | paired immunoglobin-like type 2 receptor alpha | 148.2 | 58.1 | −2.55 | <1.0E−07 |
| 215071_s_at | 848 | HIST1H2AC | histone 1, H2ac | 1220.5 | 478.0 | −2.55 | 1.1E−06 |
| 217398_x_at | 849 | GAPDH | glyceraldehyde-3-phosphate dehydrogenase | 4863.9 | 1901.0 | −2.56 | <1.0E−07 |
| 202481_at | 850 | DHRS3 | dehydrogenase/reductase (SDR family) member 3 | 581.6 | 227.2 | −2.56 | <1.0E−07 |
| 214469_at | 851 | HIST1H2AE | histone 1, H2ae | 112.7 | 43.9 | −2.57 | <1.0E−07 |
| 236213_at | 852 | NFE2L2 | Nuclear factor (erythroid-derived 2)-like 2 | 102.4 | 39.8 | −2.57 | <1.0E−07 |
| 202861_at | 853 | PER1 | period homolog 1 (Drosophila) | 1514.4 | 586.7 | −2.58 | 3.0E−07 |
| 223717_s_at | 854 | ACRBP | acrosin binding protein | 846.7 | 327.8 | −2.58 | 1.8E−06 |
| 201422_at | 855 | IFI30 | interferon, gamma-inducible protein 30 | 3272.5 | 1265.0 | −2.59 | 9.0E−07 |
| 220088_at | 856 | C5R1 | complement component 5 receptor 1 (C5a ligand) | 132.8 | 51.3 | −2.59 | <1.0E−07 |
| 202241_at | 857 | TRIB1 | tribbles homolog 1 (Drosophila) | 277.4 | 107.0 | −2.59 | <1.0E−07 |
| 201110_s_at | 858 | THBS1 | thrombospondin 1 | 62.8 | 24.1 | −2.61 | 2.3E−05 |
| 207075_at | 859 | CIAS1 | cold autoinflammatory syndrome 1 | 284.6 | 109.0 | −2.61 | <1.0E−07 |
| 204419_x_at | 860 | HBG1 | hemoglobin, gamma A | 119.9 | 45.9 | −2.61 | 4.1E−03 |
| 205442_at | 861 | MFAP3L | microfibrillar-associated protein 3-like | 159.5 | 60.8 | −2.62 | 1.5E−06 |
| 203305_at | 862 | F13A1 | coagulation factor XIII, A1 polypeptide | 823.4 | 312.3 | −2.64 | 8.0E−07 |
| 208887_at | 863 | EIF3S4 | eukaryotic translation initiation factor 3, subunit 4 delta, | 3452.5 | 1308.1 | −2.64 | <1.0E−07 |
| | | | 44 kDa | | | | |
| 208699_x_at | 864 | TKT | transketolase (Wernicke-Korsakoff syndrome) | 155.1 | 58.6 | −2.65 | <1.0E−07 |
| 203821_at | 865 | HBEGF | heparin-binding EGF-like growth factor | 207.6 | 78.3 | −2.65 | 7.0E−07 |
| 225955_at | 866 | METRNL | meteorin, glial cell differentiation regulator-like | 497.4 | 187.3 | −2.66 | <1.0E−07 |
| 208890_s_at | 867 | PLXNB2 | plexin B2 | 178.9 | 67.1 | −2.67 | <1.0E−07 |
| 220751_s_at | 868 | C5orf4 | chromosome 5 open reading frame 4 | 200.0 | 75.0 | −2.67 | <1.0E−07 |
| 242778_at | 869 | LPXN | leupaxin | 128.2 | 48.0 | −2.67 | <1.0E−07 |
| 210423_s_at | 870 | SLC11A1 | solute carrier family 11, member 1 | 267.4 | 100.2 | −2.67 | <1.0E−07 |
| 223809_at | 871 | RGS18 | regulator of G-protein signalling 18 | 841.3 | 314.8 | −2.67 | 1.0E−07 |
| 210613_s_at | 872 | SYNGR1 | synaptogyrin 1 | 221.1 | 82.7 | −2.67 | <1.0E−07 |
| 208791_at | 873 | CLU | clusterin | 199.9 | 74.7 | −2.68 | 1.5E−06 |
| 204088_at | 874 | P2RX4 | purinergic receptor P2X, ligand-gated ion channel, 4 | 190.0 | 71.0 | −2.68 | <1.0E−07 |
| 211991_s_at | 875 | HLA-DPA1 | major histocompatibility complex, class II, DP alpha 1 | 438.5 | 163.5 | −2.68 | <1.0E−07 |
| 228834_at | 876 | TOB1 | transducer of ERBB2, 1 | 277.5 | 103.4 | −2.68 | 4.5E−05 |
| 208092_s_at | 877 | FAM49A | family with sequence similarity 49, member A | 160.5 | 59.6 | −2.69 | <1.0E−07 |
| 219630_at | 878 | PDZK1IP1 | PDZK1 interacting protein 1 | 141.7 | 52.3 | −2.71 | 7.6E−04 |
| 214450_at | 879 | CTSW | cathepsin W (lymphopain) | 2011.5 | 742.2 | −2.71 | <1.0E−07 |
| 212722_s_at | 880 | PTDSR | phosphatidylserine receptor | 773.2 | 284.7 | −2.72 | <1.0E−07 |
| 242020_s_at | 881 | ZBP1 | Z-DNA binding protein 1 | 214.3 | 78.9 | −2.72 | <1.0E−07 |
| 208325_s_at | 882 | AKAP13 | A kinase (PRKA) anchor protein 13 | 140.6 | 51.6 | −2.73 | <1.0E−07 |
| 202555_s_at | 883 | MYLK | myosin, light polypeptide kinase /// myosin, light | 187.9 | 68.8 | −2.73 | 3.0E−07 |
| | | | polypeptide kinase | | | | |
| 226843_s_at | 884 | PAPD5 | PAP associated domain containing 5 | 1944.0 | 710.6 | −2.74 | <1.0E−07 |
| 242701_at | 885 | TBRG1 | Transforming growth factor beta regulator 1 | 102.6 | 37.1 | −2.77 | <1.0E−07 |
| 213872_at | 886 | C6orf62 | Chromosome 6 open reading frame 62 | 94.5 | 34.1 | −2.77 | 2.5E−05 |
| 231262_at | 887 | | Transcribed locus | 286.3 | 103.3 | −2.77 | <1.0E−07 |
| 230348_at | 888 | LATS2 | LATS, large tumor suppressor, homolog 2 (Drosophila) | 85.7 | 30.7 | −2.79 | <1.0E−07 |
| 206655_s_at | 889 | GP1BB | glycoprotein Ib (platelet), beta polypeptide | 242.9 | 86.6 | −2.80 | 4.0E−07 |
| 210754_s_at | 890 | LYN | v-yes-1 Yamaguchi sarcoma viral related oncogene homolog | 515.6 | 183.7 | −2.81 | <1.0E−07 |
| 211026_s_at | 891 | MGLL | monoglyceride lipase /// monoglyceride lipase | 252.4 | 89.5 | −2.82 | <1.0E−07 |
| 204670_x_at | 892 | HLA-DRB1 | major histocompatibility complex, class II, DR beta 1 | 4251.4 | 1499.2 | −2.84 | <1.0E−07 |
| 204187_at | 893 | GMPR | guanosine monophosphate reductase | 223.7 | 78.8 | −2.84 | 1.6E−06 |
| 212998_x_at | 894 | HLA-DQB1 | major histocompatibility complex, class II, DQ beta 1 | 365.2 | 128.1 | −2.85 | <1.0E−07 |
| 1555963_x_at | 895 | B3GNT7 | UDP-GlcNAc:betaGal beta-1,3-N- | 134.2 | 46.9 | −2.86 | <1.0E−07 |
| | | | acetylglucosaminyltransferase 7 | | | | |
| 214290_s_at | 896 | HIST2H2AA | histone 2, H2aa | 450.1 | 157.3 | −2.86 | <1.0E−07 |
| 208306_x_at | 897 | HLA-DRB5 | Major histocompatibility complex, class II, DR beta 3 | 4781.7 | 1656.4 | −2.89 | <1.0E−07 |
| 231484_at | 898 | ATP8A1 | ATPase, aminophospholipid transporter (APLT), Class I, | 543.5 | 188.0 | −2.89 | 4.8E−05 |
| | | | type 8A, member 1 | | | | |
| 207535_s_at | 899 | NFKB2 | nuclear factor of kappa light polypeptide gene enhancer in | 531.0 | 183.6 | −2.89 | 1.0E−07 |
| | | | B-cells 2 (p49/p100) | | | | |
| 204122_at | 900 | TYROBP | TYRO protein tyrosine kinase binding protein | 1294.3 | 447.0 | −2.90 | <1.0E−07 |
| 235751_s_at | 901 | LOC284013 | secretory protein LOC284013 | 97.6 | 33.7 | −2.90 | 1.0E−05 |
| 222439_s_at | 902 | THRAP3 | thyroid hormone receptor associated protein 3 | 2016.2 | 694.5 | −2.90 | <1.0E−07 |
| 238545_at | 903 | BRD7 | Bromodomain containing 7 | 1166.4 | 399.7 | −2.92 | <1.0E−07 |
| 208018_s_at | 904 | HCK | hemopoietic cell kinase | 206.2 | 69.7 | −2.96 | <1.0E−07 |
| 213787_s_at | 905 | EBP | emopamil binding protein (sterol isomerase) | 612.1 | 206.6 | −2.96 | <1.0E−07 |
| 217234_s_at | 906 | VIL2 | villin 2 (ezrin) | 346.4 | 116.3 | −2.98 | <1.0E−07 |
| 214349_at | 907 | | Hypothetical LOC388388 | 856.3 | 286.9 | −2.98 | <1.0E−07 |
| 224836_at | 908 | TP53INP2 | tumor protein p53 inducible nuclear protein 2 | 323.3 | 108.0 | −2.99 | <1.0E−07 |
| 1556545_at | 909 | | CDNA FLJ32379 fis, clone SKMUS1000030 | 1248.0 | 414.9 | −3.01 | <1.0E−07 |
| 232392_at | 910 | SFRS3 | Splicing factor, arginine/serine-rich 3 | 449.4 | 149.3 | −3.01 | 1.7E−06 |
| 205767_at | 911 | EREG | epiregulin | 79.3 | 26.3 | −3.02 | 1.0E−07 |
| 31874_at | 912 | GAS2L1 | growth arrest-specific 2 like 1 | 161.2 | 53.4 | −3.02 | <1.0E−07 |
| 205114_s_at | 913 | CCL3 | chemokine (C-C motif) ligand 3 | 555.1 | 183.5 | −3.03 | <1.0E−07 |
| 201743_at | 914 | CD14 | CD14 antigen /// CD14 antigen | 199.0 | 65.6 | −3.03 | 2.0E−07 |
| 1553043_a_at | 915 | CD300LF | CD300 antigen like family member F | 264.8 | 87.0 | −3.04 | <1.0E−07 |
| 219434_at | 916 | TREM1 | triggering receptor expressed on myeloid cells 1 | 113.4 | 37.3 | −3.04 | <1.0E−07 |
| 201125_s_at | 917 | ITGB5 | integrin, beta 5 | 494.5 | 162.4 | −3.04 | <1.0E−07 |
| 211429_s_at | 918 | SERPINA1 | serpin peptidase inhibitor, clade A, member 1 | 560.5 | 182.9 | −3.06 | 2.0E−07 |
| 205067_at | 919 | IL1B | interleukin 1, beta | 135.8 | 44.2 | −3.08 | <1.0E−07 |
| 236439_at | 920 | BCL6 | B-cell CLL/lymphoma 6 (zinc finger protein 51) | 99.6 | 32.4 | −3.08 | 4.2E−05 |
| 204614_at | 921 | SERPINB2 | serpin peptidase inhibitor, clade B (ovalbumin), member 2 | 42.6 | 13.7 | −3.11 | 1.0E−06 |
| 210982_s_at | 922 | HLA-DRA | major histocompatibility complex, class II, DR alpha | 409.7 | 130.6 | −3.14 | <1.0E−07 |
| 1555659_a_at | 923 | TREML1 | triggering receptor expressed on myeloid cells-like 1 | 181.4 | 56.7 | −3.20 | <1.0E−07 |
| 221698_s_at | 924 | CLEC7A | C-type lectin domain family 7, member A | 186.8 | 57.5 | −3.25 | <1.0E−07 |
| 209823_x_at | 925 | HLA-DQB1 | major histocompatibility complex, class II, DQ beta 1 | 292.3 | 89.9 | −3.25 | <1.0E−07 |
| 202545_at | 926 | PRKCD | protein kinase C, delta | 194.4 | 59.7 | −3.26 | <1.0E−07 |
| 203665_at | 927 | HMOX1 | heme oxygenase (decycling) 1 | 252.5 | 77.5 | −3.26 | <1.0E−07 |
| 241889_at | 928 | TA-NFKBH | T-cell activation NFKB-like protein | 84.1 | 25.8 | −3.26 | <1.0E−07 |
| 206877_at | 929 | MXD1 | MAX dimerization protein 1 | 79.8 | 24.5 | −3.26 | <1.0E−07 |
| 202672_s_at | 930 | ATF3 | activating transcription factor 3 | 525.2 | 160.7 | −3.27 | <1.0E−07 |
| 1566403_at | 931 | RNU68 | RNA, U68 small nucleolar | 122.7 | 37.4 | −3.28 | <1.0E−07 |
| 224568_x_at | 932 | MALAT1 | metastasis associated lung adenocarcinoma transcript 1 (non- | 270.3 | 81.5 | −3.32 | <1.0E−07 |
| | | | coding RNA) | | | | |
| 235102_x_at | 933 | GRAP | GRB2-related adaptor protein-like | 978.0 | 293.2 | −3.34 | <1.0E−07 |
| 230942_at | 934 | CKLFSF5 | chemokine-like factor superfamily 5 | 164.4 | 48.9 | −3.36 | <1.0E−07 |
| 208930_s_at | 935 | ILF3 | interleukin enhancer binding factor 3, 90 kDa | 1377.8 | 408.8 | −3.37 | <1.0E−07 |
| 230333_at | 936 | SAT | Spermidine/spermine N1-acetyltransferase | 244.7 | 72.1 | −3.39 | 9.5E−06 |
| 202083_s_at | 937 | SEC14L1 | SEC14-like 1 (S. cerevisiae) | 370.4 | 107.9 | −3.43 | <1.0E−07 |
| 210387_at | 938 | HIST1H2BG | histone 1, H2bg | 226.8 | 65.9 | −3.44 | <1.0E−07 |
| 228055_at | 939 | NAPSB | napsin B aspartic peptidase pseudogene | 457.9 | 133.0 | −3.44 | <1.0E−07 |
| 218559_s_at | 940 | MAFB | v-maf musculoaponeurotic fibrosarcoma oncogene homolog | 725.8 | 210.8 | −3.44 | <1.0E−07 |
| | | | B (avian) | | | | |
| 209398_at | 941 | HIST1H1C | histone 1, H1c | 217.1 | 62.8 | −3.45 | <1.0E−07 |
| 210512_s_at | 942 | VEGF | vascular endothelial growth factor | 56.8 | 16.2 | −3.50 | <1.0E−07 |
| 222760_at | 943 | ZNF703 | zinc finger protein 703 | 182.3 | 51.4 | −3.55 | <1.0E−07 |
| 217878_s_at | 944 | CDC27 | cell division cycle 27 | 236.9 | 66.7 | −3.55 | <1.0E−07 |
| 205382_s_at | 945 | DF | D component of complement (adipsin) | 475.4 | 133.8 | −3.55 | <1.0E−07 |
| 208894_at | 946 | HLA-DRA | major histocompatibility complex, class II, DR alpha | 696.7 | 196.0 | −3.56 | <1.0E−07 |
| 214073_at | 947 | CTTN | cortactin | 128.5 | 35.7 | −3.60 | <1.0E−07 |
| 235086_at | 948 | THBS1 | Thrombospondin 1 | 169.1 | 46.6 | −3.63 | 1.5E−06 |
| 202859_x_at | 949 | IL8 | interleukin 8 | 831.7 | 229.3 | −3.63 | <1.0E−07 |
| 238013_at | 950 | PLEKHA2 | pleckstrin homology domain containing, family A member 2 | 560.9 | 151.6 | −3.70 | <1.0E−07 |
| 202833_s_at | 951 | SERPINA1 | serpin peptidase inhibitor, clade A, member 1 | 125.4 | 32.2 | −3.89 | 2.0E−07 |
| 209312_x_at | 952 | HLA-DRB1 | major histocompatibility complex, class II, DR beta 1 | 4733.8 | 1216.4 | −3.89 | <1.0E−07 |
| 205237_at | 953 | FCN1 | ficolin (collagen/fibrinogen domain containing) 1 | 1909.9 | 482.9 | −3.95 | <1.0E−07 |
| 211924_s_at | 954 | PLAUR | plasminogen activator, urokinase receptor | 185.6 | 46.5 | −3.99 | <1.0E−07 |
| 204748_at | 955 | PTGS2 | prostaglandin-endoperoxide synthase 2 | 95.0 | 23.5 | −4.05 | 1.6E−06 |
| 215193_x_at | 956 | HLA-DRB1 | major histocompatibility complex, class II, DR beta 1 | 1861.9 | 455.5 | −4.09 | <1.0E−07 |
| 201101_s_at | 957 | BCLAF1 | BCL2-associated transcription factor 1 | 1195.1 | 286.5 | −4.17 | <1.0E−07 |
| 213446_s_at | 958 | IQGAP1 | IQ motif containing GTPase activating protein 1 | 278.3 | 66.4 | −4.19 | <1.0E−07 |
| 212671_s_at | 959 | HLA-DQA1 | major histocompatibility complex, class II, DQ alpha | 725.1 | 168.4 | −4.31 | 7.2E−05 |
| 210845_s_at | 960 | PLAUR | plasminogen activator, urokinase receptor | 344.1 | 79.4 | −4.33 | <1.0E−07 |
| 201360_at | 961 | CST3 | cystatin C (amyloid angiopathy and cerebral hemorrhage) | 610.2 | 136.3 | −4.48 | <1.0E−07 |
| 228986_at | 962 | OSBPL8 | oxysterol binding protein-like 8 | 347.4 | 73.4 | −4.74 | <1.0E−07 |
| 213515_x_at | 963 | HBG | hemoglobin, gamma A | 284.1 | 57.4 | −4.95 | 1.1E−03 |
| 213524_s_at | 964 | G0S2 | G0/G1switch 2 | 442.2 | 87.4 | −5.06 | <1.0E−07 |
| 208621_s_at | 965 | VIL2 | villin 2 (ezrin) | 334.2 | 52.7 | −6.34 | <1.0E−07 |
| 204018_x_at | 966 | HBA1 | hemoglobin, alpha 1 | 571.7 | 80.7 | −7.08 | <1.0E−07 |
| 211699_x_at | 967 | HBA1 | hemoglobin, alpha 1 | 574.4 | 80.7 | −7.12 | <1.0E−07 |
| 217414_x_at | 968 | HBA2 | hemoglobin, alpha 2 | 568.5 | 63.7 | −8.92 | <1.0E−07 |
| 242918_at | 969 | NASP | Nuclear autoantigenic sperm protein (histone-binding) | 122.7 | 12.0 | −10.27 | <1.0E−07 |
| 217232_x_at | 970 | HBB | hemoglobin, beta | 1881.8 | 176.7 | −10.65 | <1.0E−07 |
| 209458_x_at | 971 | HBA1 | hemoglobin, alpha 1 | 860.4 | 74.5 | −11.55 | <1.0E−07 |
| 211696_x_at | 972 | HBB | hemoglobin, beta | 2246.6 | 189.6 | −11.85 | <1.0E−07 |
| 211745_x_at | 973 | HBA1 | hemoglobin, alpha 1 | 985.4 | 78.9 | −12.50 | <1.0E−07 |
| 209116_x_at | 974 | HBB | hemoglobin, beta | 1821.4 | 98.9 | −18.41 | <1.0E−07 |
| 214414_x_at | 975 | HBA2 | hemoglobin, alpha 2 | 1686.7 | 87.5 | −19.27 | <1.0E−07 |

TABLE 6

| | Probe set | SEQ ID | | | Ratio (Grp1/ | |
|---|---|---|---|---|---|---|
| Order | Identifier | NO: | Marker Gene | Gene Description | other CIS) | p-value |
| 1 | 217800_s_at | 2 | NDFIP1 | Nedd4 family interacting protein 1 | 3.8 | <1e−07 |
| 2 | 225247_at | 298 | C19orf6 | Chromosome 19 open reading frame 6 | 3.1 | 1.E−05 |
| 3 | 213915_at | 782 | NKG7 | Natural killer cell group 7 sequence | 2.9 | <1e−07 |
| 4 | 200041_s_at | 981 | BAT1 | HLA-B associated transcript 1 | 2.5 | <1e−07 |
| 5 | 211716_x_at | 1000 | ARHGDIA | Rho GDP dissociation inhibitor (GDI) alpha | 2.5 | <1e−07 |
| 6 | 233350_s_at | 300 | TEX264 | Testis expressed sequence 264 | 2.4 | 1.E−06 |
| 7 | 219529_at | 767 | CLIC3 | Chloride intracellular channel 3 | 2.4 | <1e−07 |
| 8 | 218607_s_at | 116 | SDAD1 | SDA1 domain containing 1 | 2.3 | <1e−07 |
| 9 | 202652_at | 290 | APBB1 | Amyloid beta (A4) precursor protein-binding, family B, | 2.3 | 9.E−06 |
| | | | | member 1 | | |
| 10 | 216520_s_at | 138 | TPT1 | Tumor protein, translationally-controlled 1 | 2.3 | <1e−07 |
| 11 | 1554021 a_at | 83 | ZNF12 | Zinc finger protein 12 | 2.2 | <1e−07 |
| 12 | 204122_at | 900 | TYROBP | TYRO protein tyrosine kinase binding protein | 2.2 | <1e−07 |
| 13 | 219878_s_at | 504 | KLF13 | Kruppel-like factor 13 | 2.2 | <1e−07 |
| 14 | 216231_s_at | 242 | B2M | Beta-2-microglobulin | 2.2 | 2.E−07 |
| 15 | 219571_s_at | 106 | ZNF12 | Zinc finger protein 12 | 2.1 | <1e−07 |
| 16 | 214450_at | 879 | CTSW | Cathepsin W (lymphopain) | 2.1 | <1e−07 |
| 17 | 209050_s_at | 997 | RALGDS | Ral guanine nucleotide dissociation stimulator | 2.0 | <1e−07 |
| 18 | 207088_s_at | 992 | SLC25A11 | Solute carrier family 25, member 11 | 1.9 | <1e−07 |
| 19 | 1558215_s_at | 71 | UBTF | Upstream binding transcription factor, RNA polymerase I | 1.9 | <1e−07 |
| 20 | 200612_s_at | 982 | AP2B1 | Adaptor-related protein complex 2, beta 1 subunit | 1.9 | <1e−07 |
| 21 | 227266_s_at | 257 | FYB | FYN binding protein (FYB-120/130) | 1.9 | <1e−07 |
| 22 | 205480_s_at | 251 | UGP2 | UDP-glucose pyrophosphorylase 2 | 1.8 | <1e−07 |
| 23 | 201831_s_at | 213 | VDP | Vesicle docking protein p115 | 1.8 | 2.E−06 |
| 24 | 232535_at | 102 | | DKFZp434L201 | 1.8 | 1.E−07 |
| 25 | 244042_x_at | 269 | | Transcribed locus | 1.8 | 1.E−05 |
| 26 | 233713_at | 88 | SMYD2 | SET and MYND domain containing 2 | 1.8 | <1e−07 |
| 27 | 211947_s_at | 1001 | BAT2D1 | BAT2 domain containing 1 | 1.8 | <1e−07 |
| 28 | 212368_at | 152 | ZNF292 | Zinc finger protein 292 | 1.7 | <1e−07 |
| 29 | 217854_s_at | 573 | POLR2E | Polymerase (RNA) II (DNA directed) polypeptide E, 25 kDa | 1.7 | <1e−07 |
| 30 | 217993_s_at | 96 | MAT2B | Methionine adenosyltransferase II, beta | 1.7 | <1e−07 |
| 31 | 236562_at | 154 | ZNF439 | Zinc finger protein 439 | 1.6 | <1e−07 |
| 32 | 200033_at | 217 | DDX5 | DEAD (Asp-Glu-Ala-Asp) box polypeptide 5 | 1.6 | 1.E−07 |
| 33 | 221895_at | 225 | MOSPD2 | Motile sperm domain containing 2 | 1.6 | 8.E−07 |
| 34 | 201998_at | 987 | ST6GAL1 | ST6 beta-galactosamide alpha-2,6-sialyltranferase 1 | 1.6 | 1.E−07 |
| 35 | 209458_x_at | 971 | HBA1 /// HBA2 | Hemoglobin, alpha 1 /// alpha 2 | 1.6 | <1e−07 |
| 36 | 241838_at | 40 | | Transcribed locus | 1.5 | <1e−07 |
| 37 | 212540_at | 553 | CDC34 | Cell division cycle 34 | 1.5 | <1e−07 |
| 38 | 212926_at | 164 | SMC5L1 | SMC5 structural maintenance of chromosomes 5-like 1 | 1.5 | <1e−07 |
| 39 | 202283_at | 769 | SERPINF1 | Serpin peptidase inhibitor, clade F, member 1 | 1.5 | <1e−07 |
| 40 | 200735_x_at | 220 | NACA | Nascent-polypeptide-associated complex alpha polypeptide | 1.5 | <1e−07 |
| 41 | 207460_at | 617 | GZMM | Granzyme M (lymphocyte met-ase 1) | 1.5 | <1e−07 |
| 42 | 211699_x_at | 967 | HBA1 /// HBA2 | Hemoglobin, alpha 1 /// alpha 2 | 1.5 | <1e−07 |
| 43 | 208635_x_at | 214 | NACA | Nascent-polypeptide-associated complex alpha polypeptide | 1.5 | <1e−07 |
| 44 | 209651_at | 664 | TGFB1I1 | Transforming growth factor beta 1 induced transcript 1 | 1.5 | <1e−07 |
| 45 | 213687_s_at | 165 | RPL35A | Ribosomal protein L35a | 1.5 | <1e−07 |
| 46 | 208325_s_at | 882 | AKAP13 | A kinase (PRKA) anchor protein 13 | 1.5 | <1e−07 |
| 47 | 203375_s_at | 988 | TPP2 | Tripeptidyl peptidase II | 1.4 | 2.E−07 |
| 48 | 212063_at | 275 | CD44 | CD44 antigen | 1.4 | 3.E−07 |
| 49 | 235213_at | 76 | ITPKB | Inositol 1,4,5-trisphosphate 3-kinase B | 1.4 | <1e−07 |
| 50 | 242778_at | 869 | LPXN | Leupaxin | 0.8 | <1e−07 |
| 51 | 226843_s_at | 884 | PAPD5 | PAP associated domain containing 5 | 0.7 | <1e−07 |
| 52 | 1569599_at | 578 | SAMSN1 | SAM domain, SH3 domain and nuclear localisation signals, 1 | 0.7 | 4.E−05 |
| 53 | 207111_at | 669 | EMR1 | EGF-like module containing, mucin-like, hormone receptor- | 0.7 | <1e−07 |
| | | | | like 1 | | |
| 54 | 242918_at | 969 | NASP | Nuclear autoantigenic sperm protein (histone-binding) | 0.7 | <1e−07 |
| 55 | 1553861_at | 976 | TCP11L2 | T-complex 11 (mouse) like 2 | 0.7 | 2.E−05 |
| 56 | 1552807 a_at | 765 | SIGLEC10 | Sialic acid binding Ig-like lectin 10 | 0.6 | <1e−07 |
| 57 | 238545_at | 903 | BRD7 | Bromodomain containing 7 | 0.6 | <1e−07 |
| 58 | 202381_at | 433 | ADAM9 | ADAM metallopeptidase domain 9 (meltrin gamma) | 0.6 | 1.E−05 |
| 59 | 226982_at | 353 | ELL2 | Elongation factor, RNA polymerase II, 2 | 0.6 | 3.E−04 |
| 60 | 202083_s_at | 937 | SEC14L1 | SEC14-like 1 (S. cerevisiae) | 0.6 | <1e−07 |
| 61 | 228284_at | 844 | TLE1 | Transducin-like enhancer of split 1 | 0.6 | <1e−07 |
| 62 | 222670_s_at | 842 | MAFB | v-maf musculoaponeurotic fibrosarcoma oncogene homolog | 0.6 | <1e−07 |
| | | | | B | | |
| 63 | 243296_at | 396 | PBEF1 | Pre-B-cell colony enhancing factor 1 | 0.6 | 3.E−04 |
| 64 | 212840_at | 104 | KIAA0794 | KIAA0794 protein | 0.6 | <1e−07 |
| 65 | 1564164_at | 11 | FLJ20054 | Hypothetical protein FLJ20054 | 0.6 | <1e−07 |
| 66 | 204566_at | 990 | PPM1D | Protein phosphatase 1D magnesium-dependent, delta isoform | 0.6 | 4.E−06 |
| 67 | 226643_s_at | 184 | LOC134492 | NudC domain containing 2 | 0.6 | <1e−07 |
| 68 | 228049_x_at | 420 | | Transcribed locus | 0.6 | <1e−07 |
| 69 | 217602_at | 1010 | PPIA | Peptidylprolyl isomerase A (cyclophilin A) | 0.5 | 8.E−06 |
| 70 | 215023_s_at | 530 | PEX1 | Peroxisome biogenesis factor 1 | 0.5 | 6.E−07 |
| 71 | 1567213_at | 980 | PNN | Pinin, desmosome associated protein | 0.5 | <1e−07 |
| 72 | 209160_at | 845 | AKR1C3 | Aldo-keto reductase family 1, member C3 | 0.5 | <1e−07 |
| 73 | 205789_at | 376 | CD1D | CD1D antigen, d polypeptide | 0.5 | 5.E−04 |
| 74 | 208750_s_at | 430 | ARF1 | ADP-ribosylation factor 1 | 0.5 | 3.E−05 |
| 75 | 201151_s_at | 983 | MBNL1 | muscleblind-like (Drosophila) | 0.5 | <1e−07 |
| 76 | 212649_at | 1003 | unknown | | 0.5 | <1e−07 |
| 77 | 229733_s_at | 634 | CBX6 | Chromobox homolog 6 | 0.5 | 2.E−04 |
| 78 | 203603_s_at | 346 | ZFHX1B | Zinc finger homeobox 1b | 0.5 | 7.E−05 |
| 79 | 201110_s_at | 858 | THBS1 | Thrombospondin 1 | 0.5 | 1.E−06 |
| 80 | 1555226_s_at | 554 | C1orf43 | Chromosome 1 open reading frame 43 | 0.5 | 3.E−05 |
| 81 | 1559249_at | 593 | ATXN1 | Ataxin 1 | 0.5 | 2.E−06 |
| 82 | 204286_s_at | 502 | PMAIP1 | Phorbol-12-myristate-13-acetate-induced protein 1 | 0.5 | 1.E−07 |
| 83 | 229204_at | 34 | HP1-BP74 | Heterochromatin protein 1, binding protein 3 | 0.5 | <1e−07 |
| 84 | 219099_at | 642 | C12orf5 | Chromosome 12 open reading frame 5 | 0.5 | <1e−07 |
| 85 | 213183_s_at | 320 | CDKN1C | Cyclin-dependent kinase inhibitor 1C (p57, Kip2) | 0.5 | 2.E−06 |
| 86 | 218611_at | 560 | IER5 | Immediate early response 5 | 0.5 | <1e−07 |
| 87 | 228638_at | 348 | MGC34648 | Family with sequence similarity 76, member A | 0.4 | 2.E−07 |
| 88 | 1566403_at | 931 | RNU68 | RNA, U68 small nucleolar | 0.4 | <1e−07 |
| 89 | 227897_at | 624 | RAP2B | RAP2B, member of RAS oncogene family | 0.4 | 4.E−07 |
| 90 | 229397_s_at | 1017 | GRLF1 | Glucocorticoid receptor DNA binding factor 1 | 0.4 | 3.E−07 |
| 91 | 216609_at | 1008 | TXN | Thioredoxin | 0.4 | 1.E−07 |
| 92 | 222487_s_at | 17 | RPS27L | Ribosomal protein S27-like | 0.4 | <1e−07 |
| 93 | 228746_s_at | 704 | H41 | Hypothetical protein H41 | 0.4 | 3.E−06 |
| 94 | 230659_at | 1019 | EDEM1 | ER degradation enhancer, mannosidase alpha-like 1 | 0.4 | 3.E−04 |
| 95 | 213872_at | 886 | C6orf62 | Chromosome 6 open reading frame 62 | 0.4 | 5.E−07 |
| 96 | 228030_at | 408 | RBM6 | RNA binding motif protein 6 | 0.4 | 2.E−04 |
| 97 | 230333_at | 936 | SAT | Spermidine/spermine N1-acetyltransferase | 0.4 | 5.E−07 |
| 98 | 201694_s_at | 558 | EGR1 | Early growth response 1 | 0.3 | 6.E−07 |
| 99 | 227404_s_at | 685 | EGR1 | Early growth response 1 | 0.3 | 3.E−07 |
| 100 | 212834_at | 292 | DDX52 | DEAD (Asp-Glu-Ala-Asp) box polypeptide 52 | 0.3 | <1e−07 |
| 101 | 231193_s_at | 3 | | CDNA clone IMAGE: 4285619 | 0.3 | <1e−07 |
| 102 | 1559343_at | 978 | UBE3A | Ubiquitin protein ligase E3A | 0.3 | <1e−07 |
| 103 | 244546_at | 666 | CYCS | Cytochrome c, somatic | 0.3 | <1e−07 |
| 104 | 220494_s_at | 618 | | | 0.3 | 2.E−05 |
| 105 | 232392_at | 910 | SFRS3 | Splicing factor, arginine/serine-rich 3 | 0.3 | <1e−07 |
| 106 | 228834_at | 876 | TOB1 | Transducer of ERBB2, 1 | 0.3 | 9.E−07 |
| 107 | 214395_x_at | 1006 | EEF1D | Eukaryotic translation elongation factor 1 delta | 0.3 | 1.E−07 |
| 108 | 214041_x_at | 1004 | RPL37A | Ribosomal protein L37a | 0.2 | <1e−07 |

TABLE 3

| | CIS | Control | |
|---|---|---|---|
| Subject Characteristics | (n = 34) | (n = 28) | P-value |
| Age, y (SD) | 37 (10) | 35 (11) | 0.36 |
| Female, n (%) | 25 (74%) | 18 (64%) | 0.43 |
| Whites, n (%) | 31 (91%) | 26 (93%) | 0.81 |
| HLA-DRB1*1501 positive, n (%) | 13 (39%) | 6 (21%) | 0.15 |

TABLE 5

| Gene 1 | Gene 2 | Accuracy (%) |
|---|---|---|
| RENT1 | TIMP2 | 97 |
| ARFGAP1 | FYN | 96 |
| ARSA | FYN | 95 |
| ARFGAP1 | MAD1L1 | 91 |
| ARFGAP1 | CCDC12 | 89 |
| IVLBL | RALGDS | 87 |
| ARFGAP1 | SLC25A28 | 86 |

TABLE 7

| | | Logistic Regression | | Allele case-control | |
|---|---|---|---|---|---|
| | | Genotype | Trend | | Allele |
| | | p-value | p-value | | p-value |
| Marker | Alleles | (FDR) | (FDR) | OR | (FDR) |
| rs11079937 | A/G | 0.2957 | 0.1197 | 1.55 | 0.075928 |
| | | (0.4928) | (0.1995) | | (0.1265) |
| rs9905480 | C/T | 0.4408 | 0.8671 | 1.01 | 0.951641 |
| | | (0.5510) | (0.8671) | | (0.9516) |
| rs9303568 | C/T | 0.9449 | 0.7719 | 0.93 | 0.767137 |
| | | (0.9449) | (0.8671) | | (0.9516) |
| rs4626 | A/G | 0.0515 | 0.0142 | 0.50 | 0.00882 |
| | | (0.2200) | (0.0710) | | (0.0441) |
| rs7221352 | A/G | 0.088 | 0.0789 | 1.74 | 0.02777 |
| | | (0.2200) | (0.1972) | | (0.0694) |

TABLE 8

| | SEQ | | |
|---|---|---|---|
| Probe set | ID | Marker | |
| Identifier | NO: | Gene | Name |
| 1554021_a_at | 83 | ZNF12 | zinc finger protein 12 |
| 1555981_at | 977 | C17orf65 | chromosome 17 open reading frame 65 |
| 1559881_s_at | 979 | ZNF12 | zinc finger protein 12 |
| 200041_s_at | 981 | BAT1 | HLA-B associated transcript 1 |
| 201168_x_at | 984 | ARHGDIA | Rho GDP dissociation inhibitor (GDI) alpha |
| 206491_s_at | 991 | NAPA | N-ethylmaleimide-sensitive factor attachment protein, alpha |
| 208751_at | 995 | NAPA | N-ethylmaleimide-sensitive factor attachment protein, alpha |
| 208764_s_at | 996 | ATP5G2 | ATP synthase, H+ transporting, mitochondrial F0 complex, |
| | | | subunit C2 (subunit 9) |
| 211716_x_at | 1000 | ARHGDIA | Rho GDP dissociation inhibitor (GDI) alpha |
| 212834_at | 292 | DDX52 | DEAD (Asp-Glu-Ala-Asp) box polypeptide 52 |
| 217800_s_at | 2 | NDFIP1 | Nedd4 family interacting protein 1 |
| 218607_s_at | 116 | SDAD1 | SDA1 domain containing 1 |
| 219571_s_at | 106 | ZNF12 | zinc finger protein 12 |

TABLE 9

| | SEQ | | | | |
|---|---|---|---|---|---|
| Probe set | ID | Marker | CCP | DLDA | SVM |
| Identifier | NO: | Gene | Weight | Weight | Weight |
| 1554021_a_at | 83 | ZNF12 | 4.3768 | 2.3649 | 0.1098 |
| 1555981_at | 977 | C17orf65 | 6.3359 | 3.5256 | 0.3541 |
| 1559881_s_at | 979 | ZNF12 | 3.7162 | 3.4642 | −0.0452 |
| 200041_s_at | 981 | BAT1 | 8.0788 | 6.3002 | 0.2024 |
| 201168_x_at | 984 | ARHGDIA | 6.497 | 4.1222 | 0.6968 |
| 206491_s_at | 991 | NAPA | 3.982 | 3.8487 | 0.0581 |
| 208751_at | 995 | NAPA | 6.3683 | 3.0382 | 0.3013 |
| 208764_s_at | 996 | ATP5G2 | 5.054 | 2.0654 | −0.161 |
| 211716_x_at | 1000 | ARHGDIA | 7.6281 | 5.1502 | 0.8128 |
| 212834_at | 292 | DDX52 | −6.3979 | −3.7853 | 0.0043 |
| 217800_s_at | 2 | NDFIP1 | 4.238 | 1.405 | 0.2908 |
| 218607_s_at | 116 | SDAD1 | 5.4416 | 3.531 | 0.3313 |
| 219571_s_at | 106 | ZNF12 | 3.7024 | 1.9058 | −0.1719 |

TABLE 10A
CCP
No Std threshold = 462

| | MS | No MS | |
|---|---|---|---|
| over threshold | 12 | 0 | 1 |
| under threshold | 1 | 21 | 0.955 |
| | 0.923 | 1 | |

TABLE 10B
CCP
GAPDH Std threshold = −200

| | MS | No MS | |
|---|---|---|---|
| over threshold | 11 | 2 | 0.846 |
| under threshold | 2 | 19 | 0.905 |
| | 0.846 | 0.905 | |

TABLE 10C
CCP
ACTB Std threshold = −326

| | MS | No MS | |
|---|---|---|---|
| over threshold | 12 | 0 | 1 |
| under threshold | 1 | 21 | 0.955 |
| | 0.923076923 | 1 | |

TABLE 11A
DLDA
No Std threshold = 290

| | MS | No MS | |
|---|---|---|---|
| over threshold | 12 | 0 | 1 |
| under threshold | 1 | 21 | 0.955 |
| | 0.923 | 1 | |

TABLE 11B
DLDA
GAPDH threshold = −129

| | MS | No MS | |
|---|---|---|---|
| over threshold | 11 | 2 | 0.846 |
| under threshold | 2 | 19 | 0.905 |
| | 0.846 | 0.905 | |

TABLE 11C
DLDA
ACTB Std threshold = −201.5

| | MS | No MS | |
|---|---|---|---|
| over threshold | 12 | 0 | 1 |
| under threshold | 1 | 21 | 0.955 |
| | 0.923 | 1 | |

TABLE 12A
SVM
No Std threshold = 22.2

| | MS | No MS | |
|---|---|---|---|
| over threshold | 13 | 0 | 1 |
| under threshold | 0 | 21 | 1 |
| | 1 | 1 | |

TABLE 12B
SVM
GAPDH Std threshold = −9.3

| | MS | No MS | |
|---|---|---|---|
| over threshold | 12 | 1 | 0.923 |
| under threshold | 1 | 20 | 0.952 |
| | .0923 | 0.952 | |

TABLE 12C
SVM
ACTB Std threshold = −15.2

| | MS | No MS | |
|---|---|---|---|
| over threshold | 13 | 2 | 0.867 |
| under threshold | 0 | 19 | 1 |
| | 1 | 0.905 | |

TABLE 13

| Probe set | SEQ ID | Marker | |
|---|---|---|---|
| Identifier | NO: | Gene | Name |
| 222032_s_at | 1014 | USP7 | ubiquitin specific peptidase 7 (herpes |
| | | | virus-associated) |
| 214684_at | 1007 | MEF2A | myocyte enhancer factor 2A |
| 210081_at | 998 | AGER | advanced glycosylation end |
| | | | product-specific receptor |
| 220964_s_at | 1011 | RAB1B | RAB1B, member RAS oncogene family |
| 201864_at | 986 | GI1 | GDP dissociation inhibitor 1 |
| 210125_s_at | 999 | BANF1 | barrier to autointegration factor 1 |

TABLE 14

| Probe set | SEQ ID | CCP | DLDA | SVM |
|---|---|---|---|---|
| Identifier | NO: | Weight | Weight | Weight |
| 222032_s_at | 1014 | −7.619 | −16.193 | −0.348 |
| 214684_at | 1007 | −6.628 | −9.532 | −0.575 |
| 210081_at | 998 | 4.645 | 7.067 | 0.42 |
| 220964_s_at | 1011 | 4.885 | 9.443 | 0.472 |
| 201864_at | 986 | 5.171 | 6.959 | 0.537 |
| 210125_s_at | 999 | 6.773 | 7.182 | 0.88 |

TABLE 15A
CCP
No Std Threshold = 68

| | MS | No MS | |
|---|---|---|---|
| over threshold | 12 | 0 | 1 |
| under threshold | 1 | 21 | 0.955 |
| | 0923 | 1 | |

TABLE 15B
CCP
GAPDH Std Threshold = −20

| | MS | No MS | |
|---|---|---|---|
| over threshold | 12 | 1 | 0.923 |
| under threshold | 1 | 20 | 0.952 |
| | 0.923 | 0.952 | |

TABLE 15C
CCP
ACTB Std Threshold = −30

| | MS | No MS | |
|---|---|---|---|
| over threshold | 12 | 0 | 1 |
| under threshold | 1 | 21 | 0.955 |
| | 0.923 | 1 | |

TABLE 16A
DLDA
No Std Threshold = 53

| | MS | No MS | |
|---|---|---|---|
| over threshold | 12 | 0 | 1 |
| under threshold | 1 | 21 | 0.955 |
| | .0923 | 1 | |

TABLE 16B
DLDA
GAPDH Std threshold = −0.3

| | MS | No MS | |
|---|---|---|---|
| over threshold | 12 | 0 | 1 |
| under threshold | 1 | 21 | 0.955 |
| | 0.923 | 1 | |

TABLE 16C
DLDA
ACTB Std threshold = −10

| | MS | No MS | |
|---|---|---|---|
| over threshold | 12 | 0 | 1 |
| under threshold | 1 | 21 | 0.955 |
| | 0.923 | 1 | |

TABLE 17A
SVM
No Std threshold = 12

| | MS | No MS | |
|---|---|---|---|
| over threshold | 12 | 0 | 1 |
| under threshold | 1 | 21 | 0.955 |
| | 0.923 | 1 | |

TABLE 17B
SVM
GAPDH Std threshold = −4.3

| | MS | No MS | |
|---|---|---|---|
| over threshold | 11 | 1 | 0.917 |
| under threshold | 2 | 20 | 0.91 |
| | 0.846 | 0.952 | |

TABLE 17C
SVM
ACTB Std threshold = −7

| | MS | No MS | |
|---|---|---|---|
| over threshold | 12 | 0 | 1 |
| under threshold | 1 | 21 | 0.955 |
| | 0.923 | 1 | |

TABLE 18

| Probe set | SEQ ID | Marker | |
|---|---|---|---|
| Identifier | NO: | Gene | Name |
| 1555981_at | 977 | C17orf65 | chromosome 17 open reading frame |
| | | | 65 |
| 214123_s_at | 1005 | C4orf10 | |
| 239487_at | 1020 | FAM98A | |
| 228284_at | 844 | TLE1 | Transducin-like enhancer of split 1 |
| 207688_s_at | 993 | INHBC | |
| 208751_at | 995 | NAPA | N-ethylmaleimide-sensitive factor |
| | | | attachment protein, alpha |
| 208700_s_at | 994 | TKT | |
| 216520_s_at | 138 | TPT1 | tumor protein, translationally- |
| | | | controlled 1 |
| 1564164_at | 11 | FLJ20054 | Hypothetical protein FLJ20054 |
| 212840_at | 104 | KIAA0794 | KIAA0794 protein |
| 226643_s_at | 184 | LOC134492 | NudC domain containing 2 |
| 228638_at | 348 | MGC34648 | Family with sequence similarity 76, |
| | | | member A |

TABLE 19

| Probe set | SEQ ID | Marker | |
|---|---|---|---|
| Identifier | NO: | Gene | Name |
| 205789_at | 376 | CD1D | CD1D antigen, d polypeptide |
| 212063_at | 275 | CD44 | CD44 antigen |
| 212540_at | 553 | CDC34 | cell division cycle 34 |
| 213183_s_at | 320 | CDKN1C | Cyclin-dependent kinase inhibitor |
| | | | 1C (p57, Kip2) |
| 227259_at | 1015 | CD47 | |
| 207460_at | 617 | GZMM | granzyme M (lymphocyte met-ase 1) |
| 217602_at | 1010 | PPIA | Peptidylprolyl isomerase A |
| | | | (cyclophilin A) |

What is claimed is:

1. A method of identifying a patient with clinically isolated syndrome (CIS) at high risk of developing multiple sclerosis (MS), said method comprising:
detecting the level of expression of a marker gene within said patient, wherein said marker gene is a marker gene set forth in Table 18, Table 19, Table 1A, Table 2, Table 4, Table 8, or Table 13, or said marker gene comprises a nucleic acid of at least 10 nucleotides in length and at least 90% identity with a contiguous portion of one of SEQ ID NO:1 to SEQ ID NO:1021; and
comparing the level of expression of said marker gene to a standard control whereby a differential expression of said marker gene relative to said standard control indicates that said patient is at high risk of developing multiple sclerosis.

2. The method of claim 1, wherein said marker gene is a marker gene set forth in Table 18.

3. The method of claim 1, wherein said marker gene is a marker gene set forth in Table 19.

4. The method of claim 1, wherein said patient at high risk of developing MS is a patient with CIS that will develop MS within two years of being initially diagnosed with CIS.

5. The method of claim 1, wherein said marker gene is ZNF12 (SEQ ID NO:83), C17orf65 (SEQ ID NO:977), BAT1 (SEQ ID NO:981), ARHGDIA (SEQ ID NO:1000), NAPA (SEQ ID NO:995), ATP5G2 (SEQ ID NO:996), DDX52 (SEQ ID NO:292), NDFIP1 (SEQ ID NO:2), SDAD1 (SEQ ID NO:116), USP7 (SEQ ID NO:1014), MEF2A (SEQ ID NO:1007), AGER (SEQ ID NO:998), RAB1B (SEQ ID NO:1011), GDI1 (SEQ ID NO:986) or BANF1(SEQ ID NO:999).

6. The method of claim 1, wherein said marker gene is ZNF12 (SEQ ID NO:83), C17orf65 (SEQ ID NO:997), BAT1 (SEQ ID NO:981), ARHGDIA (SEQ ID NO:1000), NAPA (SEQ ID NO:995), ATP5G2 (SEQ ID NO:996), DDX52 (SEQ ID NO:292), NDFIP1 (SEQ ID NO:2) or SDAD1 (SEQ ID NO:116).

7. The method of claim 1, wherein said marker gene is USP7 (SEQ ID NO:1014), MEF2A (SEQ ID NO:1007), AGER (SEQ ID NO:998), RAB1B (SEQ ID NO:1011), GDI1 (SEQ ID NO:986) or BANF1 (SEQ ID NO:999).

8. The method of claim 1, wherein said marker gene is C17orf65 (SEQ ID NO:977), C4orf10 (SEQ ID NO:1005), FAM98A (SEQ ID NO:1020), TLE1 (SEQ ID NO:844), INHBC (SEQ ID NO:993), NAPA (SEQ ID NO:995), TKT (SEQ ID NO:994), TPT1 (SEQ ID NO:138), F1120054 (SEQ ID NO:11), KIAA0794 (SEQ ID NO:104), LOC134492 (SEQ ID NO:184), or MGC34648 (SEQ ID NO:348).

9. The method of claim 1, wherein said marker gene is CD1D (SEQ ID NO:376), CD44 (SEQ ID NO:275), CDC34 (SEQ ID NO:553), CDKN1C (SEQ ID NO:320), CD47 (SEQ ID NO:1015), GZMM (SEQ ID NO:617), or PPIA (SEQ ID NO:1010).

10. The method of claim 1, wherein said marker gene comprises a nucleic acid sequence at least 10 nucleotides in length having at least 90% identity with a contiguous portion a nucleic acid having the sequence of one of SEQ ID NO:1 to SEQ ID NO:1021.

11. The method of claim 1, wherein said standard control is a detected level of expression of a standard control gene in said patient.

12. The method of claim 1, wherein said standard control gene is GAPDH, 18s ribosomal subunit, beta actin (ACTB), PPP1CA, beta 2 microglobulin (B2M), HPRT1, RPS13, RPL27, RPS20 or OAZ1.

13. The method of claim 1, wherein said standard control gene is GAPDH.

14. The method of claim 1, wherein the elevated level of expression of said marker gene or the lowered level of expression of said marker gene is determined by the ratio of the level of expression of said marker gene to the level of expression of said standard control gene, whereby said ratio being approximately equal to the corresponding ratio set forth in Table 1A or Table 2 predicts development of MS within two years of being initially diagnosed with CIS.

15. The method of claim 1, wherein the elevated level of expression of said marker gene or the lowered level of expression of said marker gene is determined by a threshold expression level resulting from a statistical model.

16. The method of claim 15, wherein said statistical model is obtained using a classifier algorithm selected from a compound covariate predictor, a diagonal linear discriminant analysis, and a support vector machine.

17. A kit for use in identifying a patient with clinically isolated syndrome (CIS) at high risk of developing multiple sclerosis (MS), said kit comprising;
(i) a nucleic acid sequence having at least 90%, 91%, 92%, 93%, 94%, 95%, 96%, 97%, 98%, 99%, or 100% identity over at least a 10 nucleotide continuous region with one or more nucleic acids having SEQ ID NO:1 to SEQ ID NO:1021, or a nucleic acid complimentary thereto; and
(ii) an electronic device or computer software capable of comparing a marker gene expression level from said patient to a standard control thereby indicating whether said patient is at high risk of developing MS.
